(19) United States
(12) Patent Application Publication
(10) Pub. No.: US 2009/0215780 A1
(43) Pub. Date: Aug. 27, 2009

(54) Preparation and Use of Aryl Alkyl Acid Derivatives for the Treatment of Obesity

(75) Inventors: Roger Smith, Madison, CT (US); Derek Lowe, Hamden, CT (US); Philip Coish, North Haven, CT (US); Ann-Marie Campbell, Monroe, CT (US); Gan Wang, Wallingford, CT (US); Manoj Patel, Berlin, CT (US); Gerogiy Bondar, West Haven, CT (US)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: Bayer Pharmaceuticals Corporation, West Haven, CT (US)

(21) Appl. No.: 11/918,836

(22) PCT Filed: Apr. 18, 2006
(86) PCT No.: PCT/US2006/015194

§ 371 Date: Nov. 24, 2008

Related U.S. Application Data
(60) Provisional application No. 60/673,149, filed on Apr. 19, 2005.

(51) Int. Cl. A61K 31/5375; C07C 229/40; A61K 31/192; A61K 31/4418; C07D 213/75; C07D 295/195; A61P 3/04

(52) U.S. Cl. 514/237.5; 562/455; 514/570; 514/353; 546/306; 544/165

(57) ABSTRACT

This invention relates to certain aryl alkyl acid compounds, compositions, and methods for treating or preventing obesity and related diseases.

This application claims benefit of U.S. Provisional Application Ser. No. 60/673,149; filed on Apr. 19, 2005, the contents of which are incorporated herein by reference in their entirety.

### FIELD OF THE INVENTION

This invention relates to certain aryl alkyl acid compounds, compositions, and methods for treating or preventing obesity and related diseases.

### BACKGROUND OF THE INVENTION

Obesity, which is an excess of body fat relative to lean body mass, is a chronic disease that is highly prevalent in modern society. It is associated not only with a social stigma, but also with decreased life span and numerous medical problems, including adverse psychological development, coronary artery disease, hypertension, stroke, diabetes, hyperlipidemia, and some cancers (see, e.g., Nishina, et al., Metab. 43:554-558, 1994; Grundy and Barnett, Dis. Mon. 36:641-731, 1990; Rissanen, et al., British Medical Journal, 301:835-837, 1990).

Obesity remains a problem, and treatment has been limited. There is, therefore, a need to develop pharmaceuticals and treatment regimes effective in the alleviation of obesity.

A hallmark characteristic of obesity is an increase in white adipose tissue (WAT) mass that is largely due to accumulation of triacylglycerol. This increase in WAT mass is a key contributor to obesity-associated complications. Diacylglycerol O-acyltransferases (DGATs, EC 2.3.1.2) are membrane-bound enzymes that catalyze the terminal step of triacylglycerol biosynthesis. Two enzymes that display DGAT activity have been characterized: DGAT-1 (diacylglycerol O-acyltransferase type 1) (see, e.g., U.S. Pat. No. 6,100,077; Cases, et al., Proc. Nat. Acad. Sci. 95:13018-13023, 1998) and DGAT-2 (diacylglycerol O-acyltransferase type 2) (Cases, et al., J. Biol. Chem. 276:38870-38876, 2001). DGAT-1 and DGAT-2 do not exhibit significant protein sequence identity. Importantly, DGAT-1 null mice do not become obese when challenged with a high fat diet in contrast to wild-type littermates (Smith, et al., Nature Genetics 25:87-90, 2000). DGAT-1 null mice display reduced postprandial plasma glucose levels and exhibit increased energy expenditure, but have normal levels of serum triglycerides (Smith, et al., 2000), possibly due to the preserved DGAT-2 activity. Since DGAT-1 is expressed in the intestine and adipose tissue (Cases, et al., 1998), there are at least two possible mechanisms to explain the resistance of DGAT-1 null mice to diet-induced obesity. First, abolishing DGAT-1 activity in the intestine may block the reformation and export of triacylglycerol from intestinal cells into the circulation via chylomicron particles. Second, knocking out DGAT-1 activity in the adipocyte may decrease deposition of triacylglycerol in WAT. The phenotype of the DGAT-1 null mouse, along with the results of our studies with DGAT-1 inhibitors in diet-induced obese (DIO) mice, indicate that a DGAT-1 inhibitor has utility for the treatment of obesity and obesity-associated complications.

### DETAILED DESCRIPTION OF THE INVENTION

The invention relates to aryl alkyl acid derivatives, and pharmaceutical salts and esters thereof, that have utility in the inhibition of DGAT-1 (diacylglycerol O-acyltransferase type 1) and in the treatment of obesity and related diseases.

One embodiment of the invention is a compound of Formula (I)

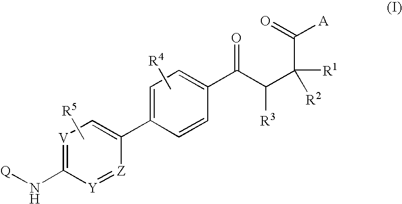

wherein
R² and R³ are both hydrogen, and R¹ is hydrogen, (C₁-C₆)alkyl, (C₁-C₆)alkoxy-(C₂-C₆)alkyl, phenoxy-(C₂-C₆)alkyl, 1-methyl-1H-indol-3-yl, bis[(C₁-C₆)alkyl]amino-(C₂-C₆)alkyl, 1-piperidinyl-(C₂-C₆)alkyl, 1-pyrrolidinyl-(C₂-C₆)alkyl, or 1-morpholinyl-(C₂₋₆)alkyl;
   or
   R¹ is R⁶(CH₂)ₘ, where m is 0 to 3, and R⁶ is phenyl optionally substituted with one or more halogen, hydroxy, (C₁-C₆)alkyl, (C₁-C₆)alkoxy, trifluoromethyl, cyano, or nitro;
   or
      R⁶ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
R³ is hydrogen, and R¹ and R² are identical and are each selected from (C₁-C₆)alkyl;
or
R³ is hydrogen, and R¹ and R² together with the carbon atom to which they are attached, form a three-to five-membered carbocyclic ring, or form a six-membered ring represented by

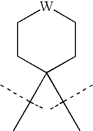

wherein W is CH₂, C(CH₃)₂, O, NH, N(CH₃), S, or SO₂;
or
R¹ is hydrogen, and R² and R³ together with the two carbon atoms to which they are attached, form a three- to six-membered carbocyclic ring;
R⁴ and R⁵ are independently selected from hydrogen, hydroxy, halo, (C₁-C₆)alkyl, (C₁-C₆)alkoxy, trifluoromethyl, and cyano;
Q is R⁷—C(O)—, where R⁷ is (C₁-C₆)alkyl optionally substituted with one or more hydroxy, (C₁-C₆)alkoxy, bis[(C₁-C₆)alkyl)amino, or fluoro;
   or
   R⁷ is R⁸(CH₂)ₙ, where n is 0 to 3, and R⁸ is phenyl optionally substituted with one or more halogen, hydroxy, (C₁-C₆)alkyl, (C₁-C₆)alkoxy, trifluoromethyl, cyano, or nitro;
      or
      R⁸ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
   or
   R⁷ is R¹⁰C(R⁹)₂, where R⁹ is methyl or ethyl,
      or
      C(R⁹)₂ is a 1,1-cyclopropyl, 1,1-cyclobutyl, 1,1-cyclopentyl, or 1,1-cyclohexyl ring;
      R¹⁰ is phenyl optionally substituted with one or more halogen, hydroxy, (C₁-C₆)alkyl, (C₁-C₆)alkoxy, trifluoromethyl, cyano, or nitro;
      or
      R¹⁰ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
   R⁷ is a fragment group selected from

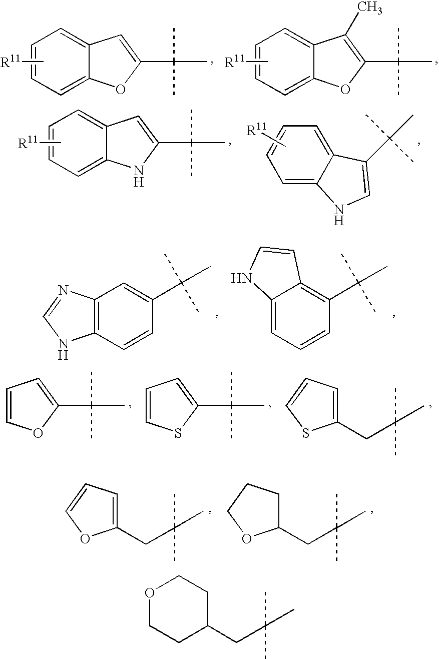

wherein R¹¹ is one or more substituents selected from halogen, hydroxy, (C₁-C₆)alkyl, (C₁-C₆)alkoxy, trifluoromethyl, cyano, and nitro;
or
Q is R¹³—N(R¹²)—C(O)—, where R¹² is hydrogen or (C₁-C₆)alkyl, and
   R¹³ is (C₁-C₆)alkyl optionally substituted with one or more hydroxy, (C₁-C₆)alkoxy, bis[(C₁-C₆)alkyl)amino, or fluoro;
   or
   R¹³ is R¹⁴(CH₂)ₚ, where p is 0 to 3, and R¹⁴ is phenyl optionally substituted with one or more halogen, hydroxy, (C₁-C₆)alkyl, (C₁-C₆)alkoxy, trifluoromethyl, cyano, or nitro;
      or
      R¹⁴ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
   or
   R¹² and R¹³ and the nitrogen atom to which they are attached form a ring fragment, selected from

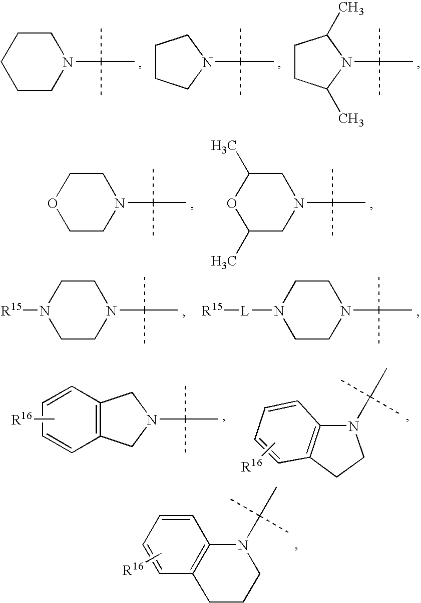

wherein L is O, C(O), or a bond;
R¹⁵ is (C₁-C₆)alkyl;
or
R¹⁵ is R¹⁷(CH₂)_{q}, where q is 0 or 1, and R¹⁷ is phenyl optionally substituted with one or more halogen, hydroxy, (C₁-C₆)alkyl, (C₁-C₆)alkoxy, trifluoromethyl, cyano, or nitro;
or
R¹⁷ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
R¹⁶ is one or more substituents selected from halogen, hydroxy, (C₁-C₆)alkyl, (C₁-C₆)alkoxy, trifluoromethyl, cyano, and nitro;
or
Q is R¹⁸—S(O)₂—, where R¹⁸ is (C₁-C₆)alkyl or benzyl;
   or
   R¹⁸ is phenyl optionally substituted with one or more halogen, hydroxy, (C₁-C₆)alkyl, (C₁-C₆)alkoxy, trifluoromethyl, cyano, or nitro;
A is OH, or NHS(O)₂—R⁹
   where R¹⁹ is (C₁-C₆)alkyl, trifluoromethyl, benzyl;
   or
   R¹⁹ is R²⁰(CH₂)ₜ, where t is 0 or 1, and R²⁰ is phenyl optionally substituted with one or more halogen, hydroxy, (C₁-C₆)alkyl, (C₂-C₆)alkoxy, trifluoromethyl, cyano, or nitro;
   or
   R¹⁹ is a fragment group selected from

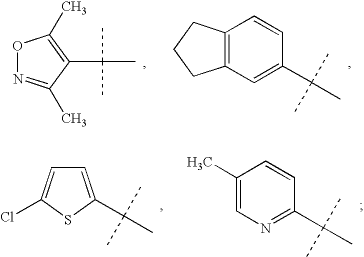

V, Y, and Z are all carbon; or
V and Y are carbon and Z is nitrogen; or
V and Z are carbon and Y is nitrogen; or
Z is carbon and V and Y are both nitrogen;
   and pharmaceutically acceptable salts and esters thereof,
   with the proviso that Formula (I) is not 4-[4′-(acetylamino)-3′-bromobiphenyl-4-yl]-4-oxobutanoic acid, 4-[4′-(acetylamino)biphenyl-4-yl]-4-oxo-2-(2-phenylethyl)butanoic acid, 4-{4′-[(3,3-dimethyl-butanoyl)amino]biphenyl-4-yl}-4-oxo-2-(2-phenylethyl)butanoic acid, or 4-oxo-4-[4′-(pentanoyl-amino)biphenyl-4-yl]-2-(2-phenylethyl)butanoic acid.

Examples of the invention may be found in the Examples described below and in the Tables. The compounds described in the Examples are intended to be representative of the invention, and it will be understood that the scope of the invention is not limited by the scope of the examples. Those skilled in the art will recognize that the invention may be practiced with variations on the disclosed structures, materials, compositions and methods, and such variations are regarded as within the ambit of the invention.

The terms identified above have the following meaning throughout:

The term “halogen” means F, Br, Cl, and I.

The terms “(C₁-C₆)alkyl” and “(C₂-C₆)alkyl” mean a linear or branched saturated hydrocarbon groups having from about 1 to about 6 carbon atoms, or from 2 to about 6 carbon atoms, respectively. The hydrocarbon group may also include a cyclic alkyl radical as part of the alkyl group. Such groups include, but are not limited to, methyl, ethyl, n-propyl, isopropyl, butyl, isobutyl, pentyl, hexyl, cyclopropyl, cyclohexyl, cyclopropyl-methyl, and cyclopentyl-methyl groups.

The term “(C₁-C₆)alkoxy” means a linear or branched saturated hydrocarbon group having from about 1 to about 6 carbon atoms, said group being attached to an oxygen atom. The oxygen atom is the atom through which the alkoxy substituent is attached to the rest of the molecule. The hydrocarbon group may also include a cyclic alkyl radical as part of the alkyl group. Such groups include, but are not limited to, methoxy, ethoxy, n-propoxy, isopropoxy, n-butoxy, n-hexyloxy, 3,3-dimethylpropoxy, cyclopropoxy, cyclopropylmethoxy, cyclopentyloxy, and the like.

The term “optionally substituted” means that the moiety so modified may have from none to up to at least the highest number of substituents indicated. Each substituent may replace any hydrogen atom on the moiety so modified as long as the replacement is chemically possible and chemically stable. When there are two or more substituents on any moiety, each substituent is chosen independently of any other substituent and can, accordingly, be the same or different.

When any moiety is described as being substituted, it can have one or more of the indicated substituents that can be located at any available position on the moiety. When there are two or more substituents on any moiety, each term shall be defined independently of any other in each occurrence.

Representative salts of the compounds of Formula (I) include the conventional non-toxic salts and the quaternary ammonium salts which are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, tartrate, thiocyanate, tosylate, and undecanoate.

Base salts include alkali metal salts such as potassium and sodium salts, alkaline earth metal salts such as calcium and magnesium salts, and ammonium salts with organic bases such as dicyclohexylamine salts and N-methyl-D-glucamine. Additionally, basic nitrogen containing groups may be quaternized with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and strearyl chlorides, bromides and iodides, aralkyl halides like benzyl and phenethyl bromides and others.

The esters in the present invention are non-toxic, pharmaceutically acceptable ester derivatives of the compounds of Formula (I). This includes, for example, ester derivatives of hydroxy-containing compounds of Formula (I) prepared with acetic, benzoic, mandelic, stearic, lactic, salicylic, hydroxynaphthoic, glucoheptonic, and gluconic acid. This also includes, for example, ester derivatives of carboxylic acid-containing compounds of Formula (I) prepared with pharmaceutically acceptable alcohols. Pharmaceutically acceptable alcohols include, but are not limited to methanol, ethanol, isopropanol, butanol, 2-methylpropanol, 2-methoxyethanol, 2-(dimethylamino)ethanol, 2-(diethylamino)ethanol, 2-(1-piperidinyl)ethanol, 2-(1-morpholinyl)ethanol, hydroxyacetic acid, N,N-dimethylglycolamide, hydroxyacetone, and the like. The compounds of Formula (I) having carboxylic acid groups may be esterified by a variety of conventional procedures well known by those skilled in the art. One skilled in the art would readily know how to successfully carry out these as well as other methods of esterification.

Sensitive or reactive groups on the compounds of Formula (I) may need to be protected during any of the above methods for forming esters, and protecting groups may be added and removed by conventional methods well known in the art.

The compounds of this invention may, either by nature of asymmetric centers or by restricted rotation, be present in the form of isomers. Any isomer may be present in which each one of any asymmetric centers is in the (R), (S), or racemic (R,S) configuration.

It will also be appreciated that when two or more asymmetric centers are present in the compounds of the invention, that several diastereomers and enantiomers of the exemplified structures will often be possible, and that pure diastereomers and pure enantiomers represent preferred embodiments. It is intended that pure stereoisomers, pure diastereomers, pure enantiomers, and mixtures thereof, are within the scope of the invention.

All isomers, whether separated, pure, partially pure, or in racemic mixture, of the compounds of this invention are encompassed within the scope of this invention. The purification of said isomers and the separation of said isomeric mixtures may be accomplished by standard techniques known in the art.

Geometric isomers by nature of substituents about a double bond or a ring may be present in cis (=Z-) or trans (=E-) form, and both isomeric forms are encompassed within the scope of this invention.

The particular process to be utilized in the preparation of the compounds of this invention depends upon the specific compound desired. Such factors as the selection of the specific moieties and the specific substituents on the various moieties, all play a role in the path to be followed in the preparation of the specific compounds of this invention. These factors are readily recognized by one of ordinary skill in the art.

For synthesis of any particular compound, one skilled in the art will recognize that the use of protecting groups may be required for the synthesis of compounds containing certain substituents. A description of suitable protecting groups and appropriate methods of adding and removing such groups may be found, for example, in Protective Groups in Organic Synthesis, Second Edition, T. W. Greene, John Wiley and Sons, New York, 1991.

In the reaction schemes below, one skilled in the art will recognize that reagents and solvents actually used may be selected from several reagents and solvents well known in the art to be effective equivalents. When specific reagents or solvents are shown in a reaction scheme, therefore, they are meant to be illustrative examples of conditions desirable for the execution of that particular reaction scheme. Abbreviations not identified in accompanying text are listed later in this disclosure under “Abbreviations and Acronyms.”

Another object of this invention is to provide methods of making the compounds of the invention. The compounds may be prepared from readily available materials by the methods outlined in the reaction scheme and Examples below, and by obvious modifications thereto.

### General Preparation of Compounds of the Invention

Preparation of the Compounds of the Present Invention Having Formula (I), may be accomplished by the general methods shown below in Reaction Schemes 1 to 9.

In Reaction Scheme 1, a coupling reaction of the compound of Formula (II) with a boronic acid or boronic ester of Formula (III), in the presence of a palladium catalyst such as PdCl₂(dppf), gives the intermediate of Formula (V). Reduction of the nitro-group in the compound of Formula (V) can be accomplished by standard means such as iron/acetic acid to provide the corresponding amino compound of Formula (VI). An alternative route to the compounds of Formula (VI) is to carry out a palladium-catalyzed coupling reaction of the compound of Formula (II) with the optionally amino-protected boronic acid or boronic ester of Formula (V), followed by deprotection, if necessary, to provide the compound of Formula (VI). The nitro or amino boronic acid/boronic ester reagents (III) and (V), respectively, are either commercially available or can be prepared from the corresponding readily available halonitrobenzenes by means well known in the art.

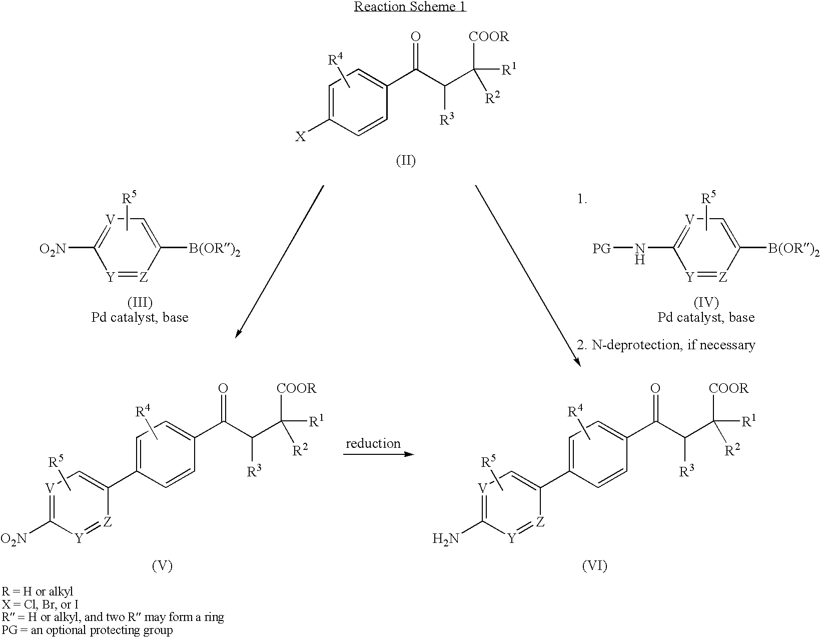

An alternative approach for the preparation of compounds of Formula (VI), that is useful when boronic acids or boronic esters of Formulas (III) and (IV) are not readily accessible, is shown in Reaction Scheme 2. Preparation of the boronic ester of Formula (VII) from the corresponding compound of Formula (II) is accomplished by reaction of (II) with a boronic ester reagent such as pinnacolborane (4,4,5,5-tetramethyl-1,3,2-dioxaborolane) to afford the intermediate of Formula (VII). This boronic ester reagent of Formula (VII) can then be coupled with the optionally protected compound of Formula (VIII), in the presence of a palladium catalyst and a base such as potassium carbonate, to give the intermediate of Formula (VI).

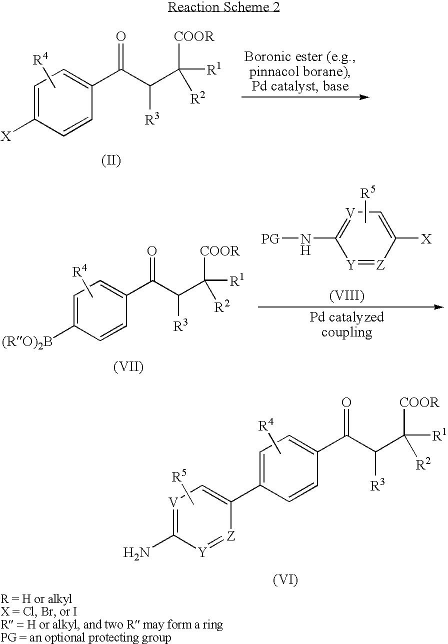

The compounds of Formula (II) may be prepared by a variety of methods described in the literature, such as in U.S. Patent Application No. 2004/0224997 and U.S. Pat. No. 5,789,434. For example, compounds of Formula (II) in which R² and R³ are both hydrogen can be prepared as shown in Reaction Scheme 3, by alkylating a substituted malonic ester of Formula (IX) with the phenacyl bromide of Formula (X), in the presence of a strong base such as sodium hydride, to give the intermediate of Formula (XI). Hydrolysis and decarboxylation of (XI) provides the compound of Formula (IIa) [(II) where R² and R³ are both H].

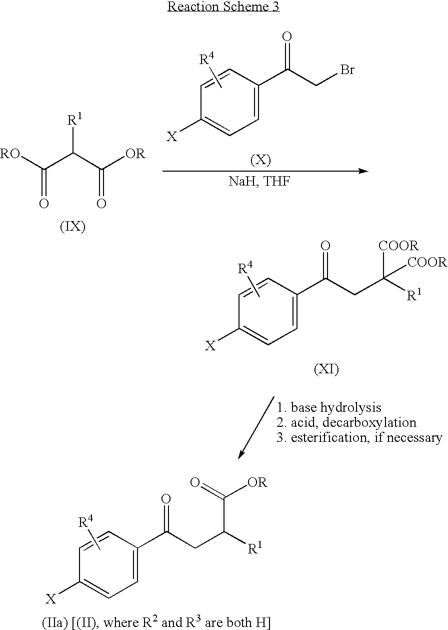

Compounds of Formula (II) can also be prepared from a readily available anhydride of Formula (XII) or an acid chloride-ester of Formula (XIII) by a Friedel-Crafts acylation reaction as shown in Reaction Scheme 4.

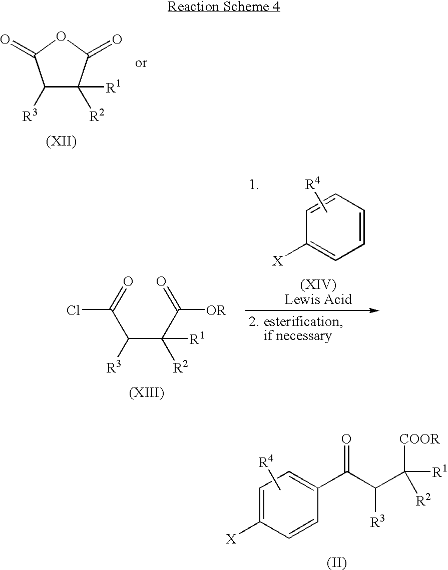

Intermediates of Formula (XIII) are either commercially available or can be prepared in a straightforward manner from readily available precursors. A general method for the preparation of Formula (XIIIa) [(XIII) where R³ is H] is shown in Reaction Scheme 5. Esterification of a substituted carboxylic acid of Formula (XV) gives a substituted ester of Formula (XVI); alkylation of the ester with t-butyl bromoacetate gives the diester of Formula (XVII). Selective removal of the t-butyl group under acidic conditions provides the monoacid monoester of Formula (XVIII) which can be converted by standard means (e.g., SOCl₂) to the ester-acid chloride of Formula (XIIIa).

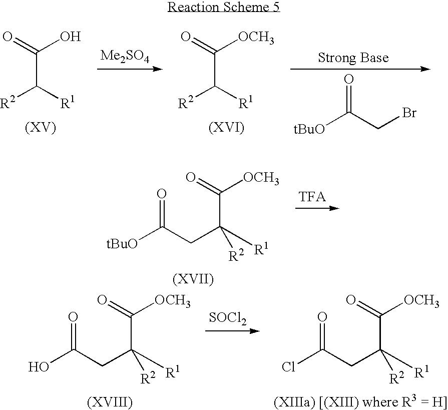

A method for the preparation of Formula (II) compounds in which R¹ is hydrogen, and R² and R³ and the two carbon atoms to which they are attached form a ring, is summarized in Reaction Scheme 6. This Reaction Scheme illustrates a general method of obtaining Formula (II) compounds where stereoisomers are possible, and specifically shows the preparation of (R,R) diastereomers of Formula (IId) and Formula (IIe).

In Reaction Scheme 6, the anhydride of Formula (XIIb) [Formula (XII) in which R¹ is hydrogen, and R² and R³ and the two carbon atoms to which they are attached form a ring] is converted in two steps to the compound of Formula (XIIIb). The method of Reaction Scheme 4 is followed to prepare the compound of Formula (IIb) from (XIIIb). Formula (IIb) may be converted to the compound of Formula (IIc) by basic hydrolysis. If desired, (IIc) may be resolved into its optical antipodes by standard means, for example, via selective crystallization of its diastereomeric salts with an optically active base such as (R)- or (S)-1-phenylethylamine, and liberating the optically purified compound by acidification of the salt. Thus, the compound of Formula (IId) can be prepared and converted to the corresponding ester of Formula (IIe).

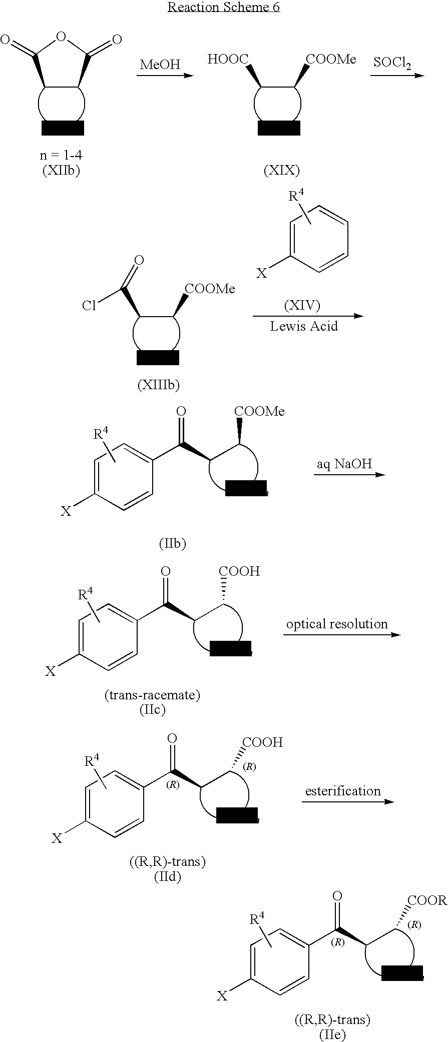

[Formula (IIb)-(IIe) represent Formula (II) where R¹=H, and R² and R³ and the two carbons to which they are attached form a ring. The ring is optionally substituted by up to two R⁸ groups, and n is 1, 2, 3, or 4.]

It is to be understood that intermediates of Formulas (IIb) to (IIe) may be individually carried on to the corresponding Formula (I) compounds by the methods outlined herein, thus allowing the preparation of different diastereomeric compounds of Formula (I).

Other compounds of Formula (II) can be prepared by methods known in the art and by the methods described herein, for example, by using compounds 1 (prepared as described in Jun, et al., Bull. Korean Chem. Soc. 9:206-209, 1988); 2 (see, e.g., methods described in U.S. Pat. No. 6,562,828); 3 and 4 (see, e.g., methods described in Carlon, et al., Org. Prep. Proc. Int 9:94-96, 1977; U.S. Pat. No. 3,256,277; Bushweller, et al., J. Org. Chem. 54:2404-2409, 1989).

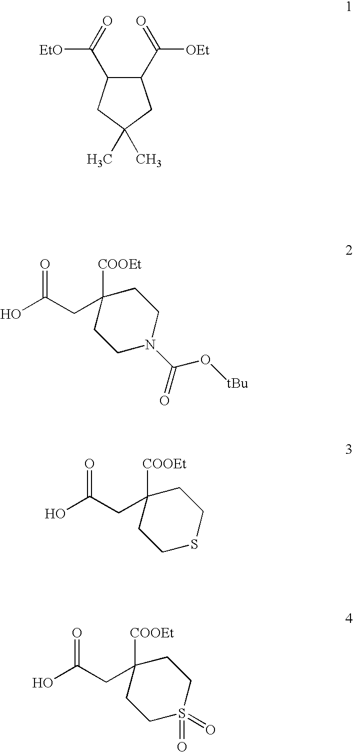

In addition, compounds of Formula (II) can be prepared by applying other methods known in the art. For example, to prepare the following specific compounds of Formula (II), designated 5 to 8, the following methods may be employed: 5 (see, e.g., WO 9615096 and U.S. Pat. No. 5,789,434); 6 (see, e.g., methods described in WO 9717317); Z (see, e.g., methods described by van der Mey, et al., J. Med. Chem. 44:2511-2522, 2001; Gaare, et al., Acta Chem. Scand. 51:1229-1233, 1997; Kuchar, et al., Coll. Czech. Chem. Commun. 51:2617-25, 1986); and 8 (see, e.g., methods described by Kawamatsu, et al., Arzneim. Forsch. 30:454-459, 1980; Bajaj, et al., J. Indian Chem. Soc. 52:1076-1078, 1975).

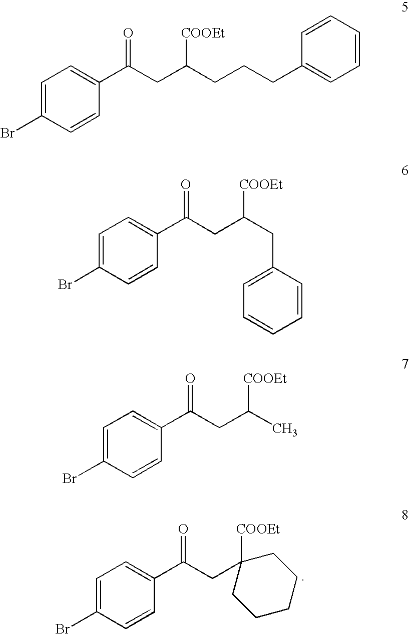

The compound of Formula (VI) prepared as described above is then converted to a compound of Formula (I) by one of the methods described in Reaction Scheme 7. For example, a compound of Formula (VI) is allowed to react with a carboxylic acid chloride or fluoride, or with a carboxylic acid plus a coupling reagent such as N,N′-dicyclohexylcarbodiimide, to form the corresponding carboxylic acid amide, and then the ester group —COOR can be hydrolyzed under standard ester hydrolysis conditions to give a compound of Formula (Ia) [(I) wherein Q is R⁷—C(O)— and A is OH].

Alternatively, the compound of Formula (VI) is allowed to react with an isocyanate derivative, R¹³—N═C═O to form the corresponding urea derivative, and then the ester group —COOR can be hydrolyzed under standard ester hydrolysis conditions to give a compound of Formula (Ib) [(I)

wherein Q is R³—NHCO— and A is OH]. Other standard methods for the formation of ureas can be applied, such as the reaction of an amine R¹³—NH₂ with carbonyldiimidazole to form an N-acyl imidazole intermediate, which is then reacted with the compound of Formula (VI) and the ester group subsequently hydrolyzed to yield a compound of Formula (Ib) [(I) wherein Q is R¹³—NH—CO— and A is OH].

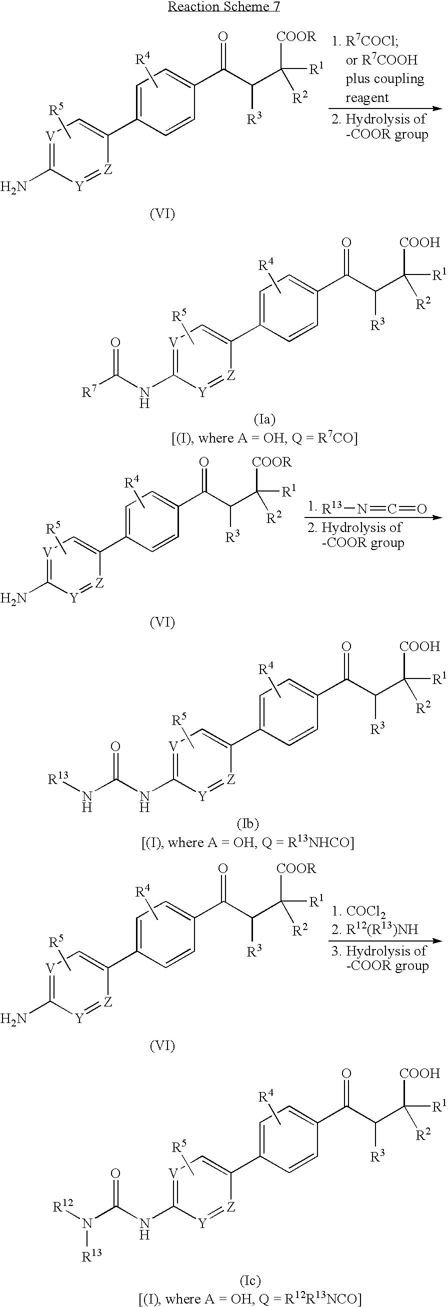

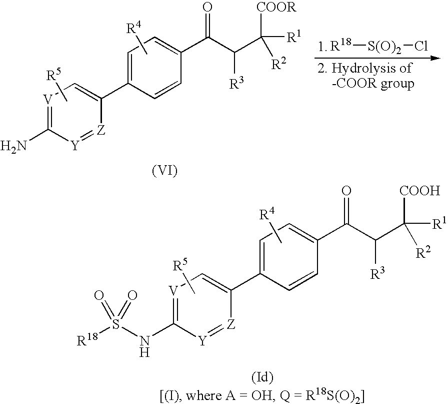

Also, the compound of Formula (VI) can be reacted with phosgene or a substitute such as triphosgene to form an isocyanate intermediate, which is then reacted with a primary or secondary amine (R¹²R¹³NH) to form the corresponding urea derivative. Then the ester group —COOR can be hydrolyzed under standard ester hydrolysis conditions to give a compound of Formula (Ic) [(I) wherein Q is R¹³—N(R²)—CO— and A is OH].

Furthermore, a compound of Formula (VI can be reacted with a sulfonyl chloride (R¹⁸SO₂Cl) to form the corresponding sulfonamide derivative, and then the ester group —COOR can be hydrolyzed under standard ester hydrolysis conditions to give a compound of Formula (Id) [a) wherein Q is R¹⁸—S(O)₂— and A is OH].

Additional compounds of Formula (I) can be prepared by the method described in Reaction Scheme 8. In this approach, the malonate ester intermediate of Formula (XXIII) is first prepared by methods analogous to those described above. This diester is then treated with a strong base such as sodium hydride, followed by an alkylating agent such as an alkyl iodide or alkyl tosylate, to give an intermediate that is hydrolyzed and decarboxylated using standard conditions to yield the compound of Formula (Ie) [(a) wherein R² and R³ are both hydrogen and A is OH).

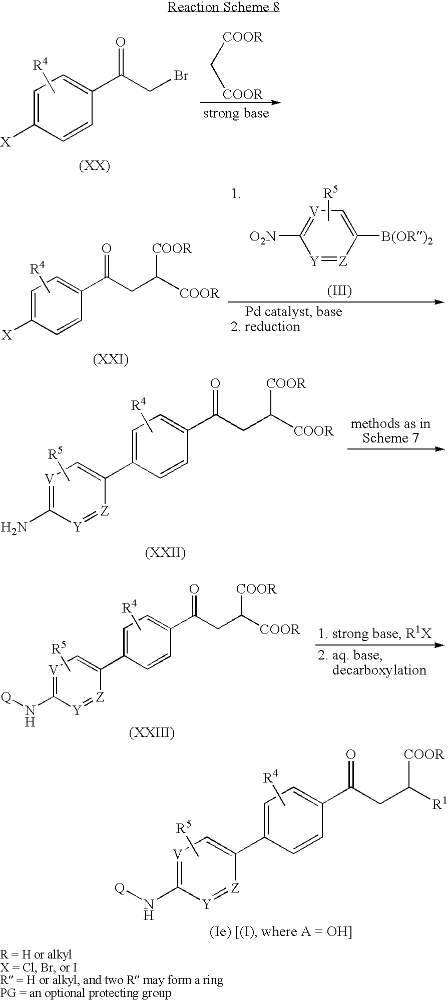

Compounds of Formula (I) wherein A is —NHS(O)₂—R¹⁹ can be prepared by treating a compound of Formula (I) wherein A is OH with an alkyl or aryl sulfonamide, in combination with a coupling reagent such as N,N′-dicyclohexylcarbodiimide, plus a base such as 4-(dimethylamino)pyridine. This methodology is described in Reaction Scheme 9.

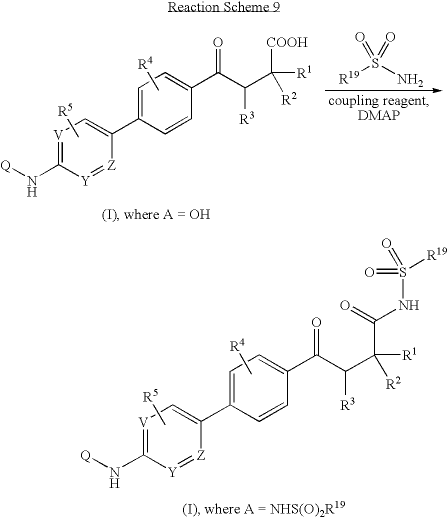

Examples of the invention may be found in the Examples described below and in the Tables. The compounds described in the Examples are intended to be representative of the invention, and it will be understood that the scope of the invention is not limited by the scope of the examples. Those skilled in the art will recognize that the invention may be practiced with variations on the disclosed structures, materials, compositions and methods, and such variations are regarded as within the ambit of the invention.

### Preparation of Compounds of the Invention

### General Information

### Mass Spectra

Chemical ionization mass spectra (CI-MS) were obtained with a Hewlett Packard 5989A mass spectrometer equipped with a Hewlett Packard 5890 Gas Chromatograph with a J & W DB-5 column (0.25 uM coating; 30 m×0.25 mm). The ion source was maintained at 250° C. and spectra were scanned from 50-800 amu at 2 sec per scan.

Liquid chromatography—electrospray mass spectra (LC-MS) data were obtained by using one of the following two methods. In the Examples and Tables provided below, the LC-MS data are given with HPLC retention times (ret. time). Except as noted otherwise, Method 1 was used.

Method 1: Hewlett-Packard 1100 HPLC equipped with a quaternary pump, a variable wavelength detector set at 254 nm, a YMC pro C-18 column (2×23 mm, 120A), and a Finnigan LCQ ion trap mass spectrometer with electrospray ionization. Spectra were scanned from 120-1200 amu using a variable ion time according to the number of ions in the source. The eluants were A: 2% acetonitrile in water with 0.02% TFA, and B: 2% water in acetonitrile with 0.018% TFA. Gradient elution from 10% B to 95% B over 3.5 minutes at a flow rate of 1.0 mL/min was used with an initial hold of 0.5 minutes and a final hold of 0.5 minutes at 95% B. Total run time was 6.5 minutes.

Method 2: Gilson HPLC system equipped with two Gilson 306 pumps, a Gilson 215 Autosampler, a Gilson diode array detector, a YMC Pro C-18 column (2×23 mm, 120 A), and a Micromass LCZ single quadrupole mass spectrometer with z-spray electrospray ionization. Spectra were scanned from 120-800 amu over 1.5 seconds. ELSD (Evaporative Light Scattering Detector) data was also acquired as an analog channel. The eluants were A: 2% acetonitrile in water with 0.02% TFA, and B: 2% water in acetonitrile with 0.018% TFA. Gradient elution from 10% B to 90% B over 3.5 minutes at a flow rate of 1.5 mu/min was used with an initial hold of 0.5 minutes and a final hold of 0.5 minutes at 90% B. Total run time was 4.8 minutes. An extra switching valve was used for column switching and regeneration.

### NMR Spectra

Routine one-dimensional NMR spectroscopy was performed on 300 MHz or 400 MHz Varian Mercury-plus spectrometers. The samples were dissolved in deuterated solvents obtained from Cambridge Isotope Labs, and transferred to 5 mm ID Wilmad NMR tubes. The spectra were acquired at 293° K. The chemical shifts were recorded on the ppm scale and were referenced to the appropriate solvent signals, such as 2.49 ppm for DMSO-d₆, 1.93 ppm for CD₃CN, 3.30 ppm for CD₃OD, 5.32 ppm for CD₂Cl₂, and 7.26 ppm for CDCl₃ for ¹H spectra; and 39.5 ppm for DMSO-d₆, 1.3 ppm for CD₃CN, 49.0 ppm for CD₃OD, 53.8 ppm for CD₂Cl₂ and 77.0 ppm for CDCl₃ for 13C spectra

### Chiral Chromatography

Chiral chromatography was carried out by using Pirkle Covalent (R,R) Whelk-O 2 10/100 from Regis Technologies as the stationary phase. The mobile phase consisted of A=Hexane (containing 0.1% TFA) and B=isopropyl alcohol (containing 0.1% TFA). The usual gradient was 10% B to 60% B over 25 minutes. In some cases, a gradient of 10 to 90% B or 50 to 90% B was used. Quantification and fraction collection was based on UV detection at 330 nm (also at 280 nm). Samples were typically dissolved in DMF prior to injection; for analytical work, these sample solutions were diluted further with methanol. For analytical work, a 4.6×250 mm column, flow rate=1 mL/min, and Shimadzu analytical HPLC were used. For preparative work, a 20×250 mm column, flow rate=25 mL/min, and Gilson HPLC were used, with a typical injected sample quantity of 50 mg.

### Abbreviations and Acronyms

When the following abbreviations are used throughout the disclosure, they have the following meaning:
CDCl₃ deuterated chloroform
DCE dichloroethane
DCM dichloromethane
DMF N,N-dimethylformamide
DMSO dimethyl sulfoxide
DMSO-d₆ deuterated dimethyl sulfoxide-
EtOAc ethyl acetate
h hour(s)
GC-MS gas chromatography—mass spectrometry
HPLC high pressure liquid chromatography
LC-MS liquid chromatography—mass spectrometry
MeOH methanol
min minutes
MS mass spectroscopy
NMR nuclear magnetic resonance
PdCl₂(dppf) 1,1′-bis(diphenylphosphino)ferrocene] dichloropalladium(II)
p.o. orally administered
PS-DIEA diisopropylaminomethyl polystyrene (purchased from Argonaut Technologies, San Carlos, Calif., USA)
Rf TLC retention factor
rt room temperature
RT retention time
TFA trifluoroacetic acid
TFFH tetramethyfluoro-formamidinium hexafluorophosphate (purchased from Advanced Chemtech, Louisville, Ky., USA)
THF tetrahydrofuran
TLC thin layer chromatography

### EXAMPLES

### Example 1

### Preparation of 2-benzyl-4-oxo-4-[4′-(pentanoylamino)-1,1′-biphenyl-4-yl]-butanoic acid

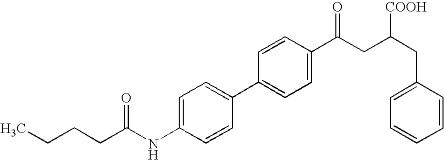

### Step 1. Preparation of diethyl 2-benzyl-2-[2-(4-bromophenyl)-2-oxoethyl]malonate

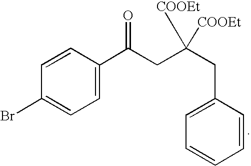

The procedure was based on a procedure described in U.S. Pat. No. 5,789,434. To a 500 mL 3-neck round-bottom flask fitted with an argon inlet, septum, and an addition funnel was added sodium hydride (95%, 1.05 g, 44 mmol), followed by anhydrous tetrahydrofuran (30 mL). The suspension was then cooled to 0° C., and diethyl benzylmalonate (10.0 g, 40 mmol) in tetrahydrofuran (20 mL) was added dropwise over 20 nm in. The cooling bath was removed, and the reaction mixture was allowed to warm to rt and then stirred for 45 min. A solution of 2,4′-dibromoacetophenone (11.1 g, 40 mmol) in tetrahydrofuran (40 mL) was then added to the stirred mixture. The reaction mixture was stirred at rt under argon overnight, then the reaction vessel was cooled in an ice bath while 75 mL water was added cautiously dropwise. The aqueous layer was extracted with 200 mL dichloromethane. The combined organic phase was washed with 10% aqueous hydrochloric acid (50 mL) and saturated aqueous sodium bicarbonate (50 mL), dried over sodium sulfate, and concentrated under reduced pressure to afford diethyl 2-benzyl-2-[2-(4-bromophenyl)-2-oxoethyl]malonate as a red oil (16.8 g, 94.3%). TLC Rf=0.85 (1:4 ethylacetate/hexane); LC-MS RT=3.49 min (method 2), m/z 447 (MH⁺); ¹H NMR (300 MHz, CDCl₃) δ 7.79 (d, 2H), 7.61 (d, 2H), 7.19 (m, 3H), 6.90 (m, 2H), 4.21 (m, 4H), 3.50 (s, 2H), 3.40 (s, 2H), 1.22 (m, 6H).

### Step 2. Preparation of ethyl 2-benzyl-4-(4-bromophenyl)-4-oxobutanoate

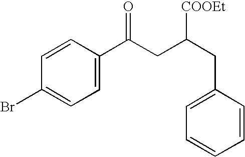

To a solution of diethyl 2-benzyl-2-[2-(4-bromophenyl)-2-oxoethyl]malonate (16.8 g, 37.6 mmol) in acetone (18.5 mL) and ethanol (17.0 mL) was added a 1 N aqueous solution of sodium hydroxide (37.6 mL, 37.6 mmol), and the resulting solution was heated at 50° C. for 3 h. Solvent was then removed under reduced pressure and the residue was dried under high vacuum for 1 h. The residue was then re-dissolved in dichloroethane (46 mL) and heated at 80° C. for 2.5 h. The mixture was then cooled to rt, diluted with ethyl acetate, and washed with water. The organic layer was dried over anhydrous sodium sulfate and concentrated under reduced pressure to afford ethyl 2-benzyl-4-(4-bromophenyl)-4-oxobutanoate as a red oil (10.0 g, 71.5%). TLC Rf=0.80 (1:4 ethyl acetate/hexane); LC-MS RT=3.37 min (method 2), m/z 375 (MH⁺); ¹H NMR (300 MHz, CDCl₃) δ 7.68 (d, 2H), 7.50 (d, 2H), 7.19 (m, 5H), 4.05 (m, 2H), 3.25 (m, 2H), 3.00 (m, 1H), 2.80 (m, 2H), 1.11 (t, 3H).

### Step 3. Preparation of ethyl 2-benzyl-4-(4′-nitro-1,1′-biphenylyl-4-yl)-4-oxobutanoate

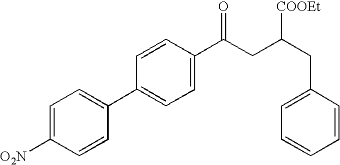

A mixture of ethyl 2-benzyl-4-(4-bromophenyl)-4-oxobutanoate (3.75 g, 10.0 mmol), 4-nitro-phenyl boronic acid (1.8 g, 11 mmol), and 2 N aqueous sodium carbonate (25 mL) in toluene/dioxane (65 mL/20 mL) was degassed by a flow of argon for 20 min. Then, [1,1′-bis(diphenylphosphino)-ferrocene]dichloro palladium(I) (1:1 complex with dichloromethane, 400 mg, 0.5 mmol) was added, and this reaction mixture was heated at 85° C. for 5 h. The reaction mixture was cooled to rt, filtered, and the organic layer was washed with water (50 mL), dried over sodium sulfate, and concentrated under reduced pressure to afford ethyl 2-benzyl-4-(4′-nitro-1,1′-biphenyl-4-yl)-4-oxobutanoate as a black gum (3.56 g, 85%), which was used without purification in the next step. TLC Rf=0.30 (1:5 ethyl acetate/hexane); LC-MS RT=3.54 min (method 2), m/z 418 (MH⁺); ¹H NMR (300 MHz, CDCl₃) δ 8.25 (d, 2H), 8.0 (d, 2H), 7.68 (m, 4H), 7.20 (m, 5H), 4.05 (m, 2H), 3.40 (m, 2H), 3.10 (m, 1H), 2.80-2.90 (m, 2H), 1.11 (t, 3H).

### Step 4. Preparation of ethyl 4-(4′-amino-1,1′-biphenyl-4-yl)-2-benzyl-4-oxobutanoate

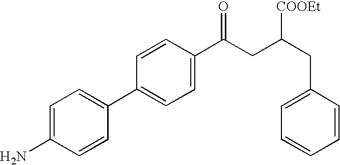

To a solution of ethyl 2-benzyl-4-(4′-nitro-1,1′-biphenylyl)-4-oxobutanoate (3.87 g, 9.30 mmol) in 85% ethanol (160 mL) was added iron powder (5.0 g, 89 mmol), followed by 2 N aqueous hydrochloric acid (5.0 mL), and the resulting mixture was heated at reflux for 3 h. The mixture was then cooled to rt, filtered through a pad of celite, and extracted with ethyl acetate. The combined organic phase was then dried over anhydrous sodium sulfate and concentrated under reduced pressure to afford ethyl 4-(4′-amino-1,1′-biphenyl-4-yl)-2-benzyl-4-oxobutanoate as a brown solid (3.0 g, 84%). TLC Rf=0.50 (1:4 ethyl acetate/hexane); LC-MS RT=2.80 min (method 2), m/z 388 (MH⁺); ¹H NMR (300 MHz, CDCl₃) δ 7.90 (m, 2H), 7.70-7.35 (m, 6H), 7.30-7.20 (m, 3H), 6.70 (m, 2H), 4.05 (m, 2H), 3.40 (m, 2H), 3.10-2.80 (m, 3H), 1.11 (t, 3H).

### Step 5. Preparation of 2-benzyl-4-oxo-4-oxo-4-[4′-(pentanoylamino)-1,1′-biphenyl-4-yl]butanoic acid

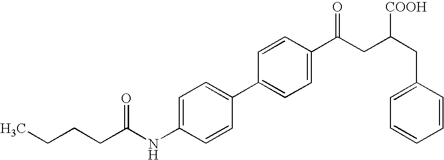

To a solution of ethyl 4-(4′-amino-1,1′-biphenyl-4-yl)-2-benzyl-4-oxobutanoate (30 mg, 0.078 mmol) and valeryl chloride (13.9 mg, 0.116 mmol) in dichloromethane (1.0 mL) was added PS-DIEA (43 mg, 0.16 mmol), and the resulting suspension was mixed by orbital shaking at rt overnight. The mixture was then filtered, and the filtrate was concentrated under reduced pressure. The solid residue was re-dissolved in 1 mL methanol/tetrahydrofuran (1:1), and 1 N aqueous solution of sodium hydroxide (0.3 mL) was added. This reaction mixture was shaken at rt overnight, then 2 N aqueous hydrochloric acid (0.2 mL) was added, and the mixture was concentrated under reduced pressure. The solid residue was dissolved in methanol and purified by preparative reverse-phase HPLC (water/acetonitrile gradient, containing 0.1% TFA). The product 2-benzyl-4-oxo-4-[4′-(pentanoylamino)-1,1′-biphenyl-4-yl]butanoic acid was obtained as a white solid (20 mg, 59%). LC-MS RT=3.14 min (method 2), m/z 444 (MH⁺); ¹H NMR (300 MHz, DMF-d₇) δ 12.60 (s, 1H), 10.10 (s, 1H), 8.02 (d, 2H), 7.85 (m, 4H), 7.75 (d, 2H), 7.32 (m, 4H), 7.10 (m, 1H), 3.37 (m, 1H), 3.12 (m, 2H), 2.90 (m, 2H), 2.40 (t, 2H), 1.62 (m, 2H), 1.37 (m, 2H), 0.94 (t, 3H).

### Example 2

### Preparation of 4-oxo-4-[4′-(pentanoylamino)-1,1′-biphenyl-4-yl]-2-(2-phenylethyl)butanoic acid

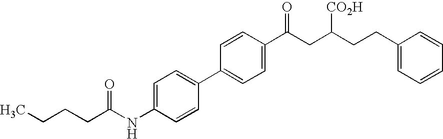

### Step 1. Preparation of ethyl 4-oxo-4-[4′-(pentanoylamino)-1,1′-biphenyl-4-yl]-2-(2-phenylethyl)butanoate

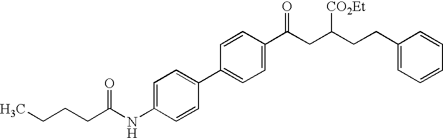

To a solution of ethyl 4-(4′-amino-1,1′-biphenyl-4-yl)-4-oxo-2-(2-phenylethyl)butanoate (4.63 g, 11.5 mmol, prepared as described in US 2004/0224997) and valeryl chloride (1.67 g, 13.8 mmol) in dichloromethane (70 mL) was added poly-4-vinyl pyridine (3.8 g, 34.6 mmol). The resulting suspension was stirred at rt for 3 h and then filtered. The filtrate was washed with water, dried over anhydrous sodium sulfate, and concentrated under reduced pressure to afford ethyl 4-oxo-4-[4′-(pentanoylamino)-1,1′-biphenyl-4-yl]-2-(2-phenylethyl)butanoate (5.47 g, 97%). LC-MS RT=3.83 min, m/z 486.5 (MH⁺); ¹H NMR (300 MHz, CDCl₃) δ 0.91 (t, 3H), 1.23 (t, 3H), 1.33-1.41 (m, 2H), 1.68-1.75 (m, 2H), 1.82-2.01 (m, 2H), 2.29 (t, 2H), 2.64 (t, 2H), 3.05-3.18 (m, 2H), 3.41-3.48 (m, 1H), 4.10 (q, 2H), 7.15-7.24 (m, 6H), 7.51-7.62 (m, 6H), 7.94 (d, 2H).

### Step, 2. Preparation of 4-oxo-4-[4′-(pentanoylamino)-1,1′-biphenyl-4-yl]-2-(2-phenylethyl)butanoic acid

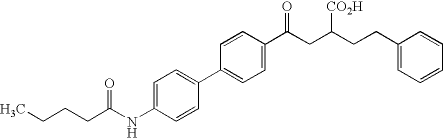

To a solution of ethyl 4-oxo-4-[4′-(pentanoylamino)-1,1′-biphenylyl-4-yl]-2-(2-phenylethyl)butanoate (5.23 g, 10.8 mmol) in methanol (52 mL) was added a 1.0 N aqueous solution of sodium hydroxide (37.7 mL, 37.7 mmol). Tetrahydrofuran (52 mL) was added to dissolve precipitate that formed during stirring. The mixture was heated at 50° C. for 2 h, and was then concentrated by rotary evaporation. The residue was quickly treated dropwise with 1.0 N aqueous hydrochloric acid giving a thick yellow slurry which was then filtered. The solid was washed with water and hexane and was dried under reduced pressure at 40° C. to afford 4-oxo-4-[4′-(pentanoylamino)-1,1′-biphenyl-4-yl]-2-(2-phenylethyl)butanoic acid (4.8 g, 97%). LC-MS RT=3.44 min, m/z 458.7 (MH⁺); ¹H NMR (300 MHz, DMSO-d₆) δ 0.94 (t, 3H), 1.26-1.40 (m, 2H), 1.54-1.62 (m, 2H), 1.79-1.96 (m, 2H), 2.31 (t, 2H), 2.67 (t, 2H), 2.82-2.90 (m, 1H), 3.20 (dd, 1H), 3.38-3.46 (m, 1H), 7.15-7.28 (m, 5H), 7.70 (s, 4H), 7.77 (d, 2H), 8.00 (d, 2H), 10.01 (s, 1H), 12.1 (s, 1H).

### Example 3

### Preparation of the sodium salt of 4-oxo-4-[4′-(pentanoylamino)-1,1′-biphenyl-4-yl]-2-(2-phenylethyl)butanoic acid

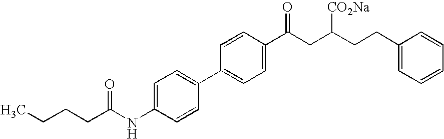

To a solution of 4-oxo-4-[4′-(pentanoylamino)-1,1′-biphenyl-4-yl]-2-(2-phenylethyl)-butanoic acid (900 mg, 1.97 mmol, prepared as described in Example 2) in ethanol (22 mL) at 40° C. was added a solution of 1.0 N aqueous sodium hydroxide (1.93 mL, 1.93 mmol), and the resulting solution was stirred for 1 h. The mixture was concentrated under reduced pressure, and the resulting solid was further dried under reduced pressure at 40° C. to afford sodium 4-oxo-4-[4′-(pentanoylamino)-1,1′-biphenyl-4-yl]-2-(2-phenylethyl)butanoate (802 mg, 85%). LC-MS RT=3.43 min., mm/z 458.6 (MH⁺); ¹H NMR (300 MHz, DMSO-d₆) δ 0.96 (t, 3H), 1.30-1.1.36 (m, 2H), 1.54-1.63 (m, 3H), 1.79-1.83 (m, 1H), 2.32 (t, 2H), 2.62-2.79 (m, 4H), 3.43 (m, 1H), 7.08-7.25 (m, 5H), 7.62-7.75 (m, 6H), 7.97 (d, 2H), 10.21 (s, 1H).

### Example 4

### Preparation of the Individual Enantiomers of 4-oxo-4-[4′-(pentanoyl-amino)-1,1′-biphenyl-4-yl]-2-(2-phenylethyl)butanoic acid

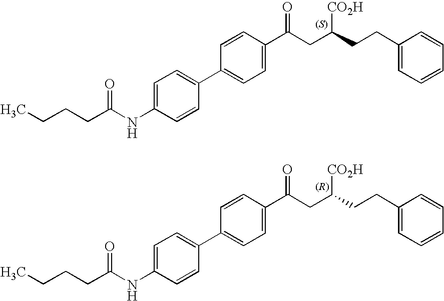

A sample of racemic 4-oxo-4-[4′-(pentanoylamino)-1,1′-biphenyl-4-yl]-2-(2-phenylethyl) butanoic acid (prepared as described in Example 2) was separated into its two individual enantiomers by preparative chiral chromatography, using a Pirkle Covalent (R,R)Whelk-O-2 10/100,250×4.5 mm column (obtained from Regis Technologies, Inc.), eluting with a 10 to 90% isopropanol/hexane gradient. The two enantiomers were each isolated in approximately 30% yield, in >90% enantiomeric purity; LC-MS and ¹H NMR analytical data were essentially as described above for the racemic compound.

### Example 5

### Preparation of 4-[4′-({[(3,4-dimethylphenyl)amino]carbonyl}amino)-1,1′-biphenyl-4-yl]-4-oxo-2-(2-phenylethyl)butanoic acid

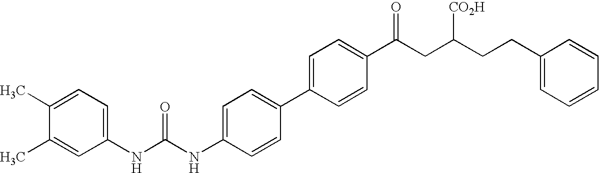

A mixture of ethyl 4-(4′-amino-1,1′-biphenyl-4-yl)-4-oxo-2-(2-phenylethyl)butanoate (25 mg, 0.062 mmol, prepared as described in US 2004/0224997), 3,4-dimethylphenyl isocyanate (18 mg, 0.120 mmol), and dichloromethane (1 mL) was stirred at rt overnight. The mixture was concentrated under reduced pressure and the residue was dissolved in tetrahydrofuran (0.30 mL) and methanol (0.30 mL). Aqueous sodium hydroxide (1 N, 0.20 mL, 0.20 mmol) was then added. The resulting mixture was stirred overnight, filtered, and concentrated. The residue was purified by preparative reverse-phase HPLC (water/acetonitrile gradient, containing 0.1% TFA) to give 4-[4′-({[(3,4-dimethyl-phenyl)amino]carbonyl}amino)-1,1′-biphenyl-4-yl]-4-oxo-2-(2-phenylethyl)butanoic acid as a white solid (6 mg, 19% yield over two steps). LC-MS RT=3.78 min, m/z 521.2; ¹H NMR (DMSO-d₆) δ 1.75-1.98 (m, 2H), 2.17 (s, 3H), 2.19 (s, 3H), 2.61-2.72 (m, 2H), 2.78-2.91 (m, 1H), 3.15 (dd, 1H), 3.34 (dd, 1H), 7.01 (d, 1H), 7.12-7.34 (m, 7H), 7.57 (d, 2H), 7.69 (d, 2H), 7.99 (d, 2H), 8.04 (d, 2H), 8.64 (br s, 1H), 8.93 (br s, 1H), 12.23 (br s, 1H).

### Example 6

### Preparation of 4-{4′-[(butylsulfonyl)amino]-1,1′-biphenyl-4-yl}-4-oxo-2-(2-phenylethyl) butanoic acid

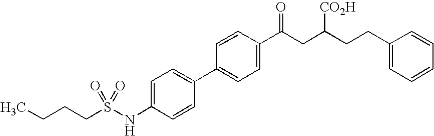

To a solution of ethyl 4-(4′-amino-1,1′-biphenyl-4-yl)-4-oxo-2-(2-phenylethyl)butanoate (38.4 mg, 0.096 mmol, prepared as described in US 2004/0224997) and 1-butanesulphonyl chloride (16.5 mg, 0.105 mmol) in dichloromethane (0.75 mL) was added polyvinyl pyridine (32 mg, 0.29 mmol). The resulting suspension was stirred at rt for 16 h, and was then filtered. The filtrate was washed with water and concentrated under reduced pressure. The mixture was then dissolved in methanol (0.6 mL) and tetrahydrofuran (0.6 mL), and a 1.0 N aqueous solution of sodium hydroxide (0.4 mL, 0.4 mmol) was added. The mixture was heated at 50° C. for 2 h, and then concentrated under reduced pressure. The residue was purified by preparative reverse-phase HPLC (water/acetonitrile gradient, containing 0.1% TFA) to afford 4-{4′-[(butylsulfonyl)amino]-1,1′-biphenyl-4-yl})-4-oxo-2-(2-phenyl-ethyl)butanoic acid (12.6 mg, 27%). LC-MS RT=4.04 min, m/z 494.2 (MH⁺); ¹H NMR (300 MHz, CDCl₃) δ 0.88 (t, 3H), 1.32-1.38 (m, 2H), 1.73 (m, 2H), 1.89-1.96 (m, 1H), 2.08-2.12 (1, 1H), 2.73 (t, 2H), 3.02-3.17 (m, 4H), 3.47-3.53 (m, 1H), 6.81 (s, 1H), 7.13-7.28 (m, 7H), 7.47 (d, 2H), 7.56 (d, 2H), 7.95 (d, 2H).

### Example 7

### Preparation of 4-[4′-({[1-(4-methoxyphenyl)cyclopropyl]carbonyl}amino)-1,1′-biphenyl-4-yl]-4-oxo-2-(2-phenylethyl)butanoic acid

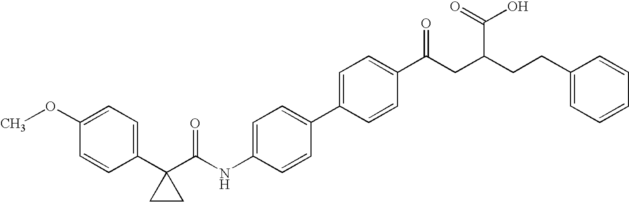

In a 8-mL screw-cap vial, 1-(4-methoxyphenyl)cyclopropanecarboxylic acid (100 mg, 0.52 mmol), TFFH (151 mg, 0.57 mmol), and PS-DIEA (loading level: 3.50 mmol/g, 743 mg, 2.6 mmol) were combined in 8 mL 1,2-dichloroethane and heated at 35° C. with orbital shaking overnight. The formation of acyl fluoride was monitored by LC-MS. To the mixture, methyl 4-(4′-amino-1,1′-biphenyl-4-yl)-4-oxo-2-(2-phenylethyl)butanoate (0.9 equivalent, 181 mg, 0.47 mmol, prepared as described in US 2004/0224997) was added and the reaction mixture was again heated at 35° C. with orbital shaking overnight. The mixture was cooled to rt, and filtered through a filter tube (polypropylene frit), and the filtrate was evaporated under reduced pressure. The crude product residue was redissolved in 1 mL of MeOH and purified by preparative reverse-phase HPLC (water/acetonitrile gradient, containing 0.1% TFA). The methyl ester obtained was hydrolyzed as previously described, and the product was purified by preparative reverse-phase HPLC (water/acetonitrile gradient, containing 0.1% TFA) to give 37 mg of 4-[4′-({[1-(4-methoxyphenyl)-cyclopropyl]carbonyl}amino)1,1′-biphenyl-4-yl]-4-oxo-2-(2-phenylethyl)butanoic acid (Yield: 13%). ¹H NMR (400 MHz, DMSO-d₆) δ 12.20 (bs, 1H), 9.00 (s, 1H), 8.00 (d, 2H), 7.80 (d, 2H), 7.65 (s, 4H), 7.15-7.40 (m, 7H), 6.95 (d, 2H), 3.75 (s, 3H), 3.45 (q, 1H), 3.20 (m, 1H), 2.85 (m, 1H), 2.70 (m, 2H), 1.85 (m, 2H), 1.40 (t, 2H), 1.10 (t, 2H); LC-MS (method 2) m/z 548.5 (MH⁺), ret. time 3.76 min.

### Example 8

### Preparation of 4-{4′-[(4-methoxybenzoyl)amino]-3-methyl-1,1′-biphenyl-4-yl}-4-oxo-2-(2-phenylethyl)butanoic acid

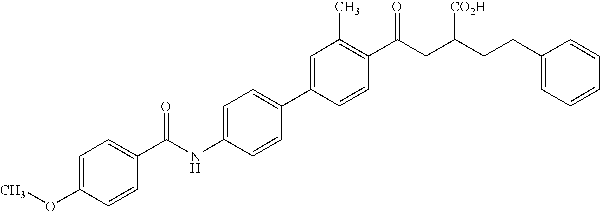

A mixture of ethyl 4-(4′-amino-3-methyl-1,1′-biphenylyl)-4-oxo-2-(2-phenylethyl)-butanoate (25 mg, 0.060 mmol, prepared as described in US 2004/0224997), 4-methoxybenzoyl chloride (20 mg, 0.12 mmol), diisopropylaminomethyl polystyrene (PS-DIEA) (0.050 g, 0.18 mmol), and dichloromethane (1 mL) was stirred at rt overnight. The mixture was filtered, and the filtrate was concentrated under reduced pressure. The residue was dissolved in tetrahydrofuran (0.30 mL) and methanol (0.30 mL), and 1 N aqueous sodium hydroxide (0.20 mL, 0.20 mmol) was added. The resulting mixture was stirred overnight, filtered, and concentrated under reduced pressure. The residue was purified by preparative reverse-phase HPLC (water/acetonitrile gradient, containing 0.1% TFA) to give 4-{4′-[(4-meth-oxybenzoyl)amino]-3-methyl-1,1′-biphenyl-4-yl}-4-oxo-2-(2-phenylethyl)butanoic acid as a white solid (9.6 mg, 31% yield for two steps). LC-MS RT=3.63 min, m/z 522.2 (MH⁺); ¹H NMR (CDCl₃) δ 1.85-2.02 (m, 1H), 2.04-2.21 (m, 1H), 2.57 (s, 3H), 2.72-2.81 (m, 2H), 3.09 (dd, 1H), 3.14-3.22 (m, 1H), 3.48 (dd, 1H), 4.91 (s, 3H), 6.99 (d, 2H), 7.16-7.38 (m, 5H), 7.43-7.52 (m, 2H), 7.62 (d, 2H), 7.72-7.80 (m, 3H), 7.81-7.93 (m, 3H).

### Example 9

### Preparation of 4-{3-methyl-4′-[({[4-(trifluoromethyl)-phenyl]-aminocarbonyl)-amino]-1,1′-biphenyl-4-yl}-4-oxo-2-(2-phenylethyl)butanoic acid

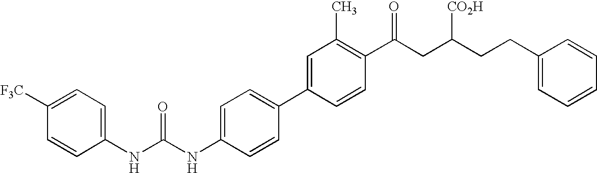

A mixture of ethyl 4-(4′-amino-3-methyl-1,1-biphenyl-4-yl)-4-oxo-2-(2-phenylethyl) butanoate (0.025 g, 0.060 mmol, prepared as described in US 2004/0224997), 4-trifluoromethylphenyl isocyanate (16 mg, 0.12 mmol), and dichloromethane (1 mL) was stirred at rt overnight. The mixture was concentrated under reduced pressure. The residue was dissolved in tetrahydrofuran (0.30 mL) and methanol (0.30 mL), and 1 N aqueous sodium hydroxide (0.20 mL, 0.20 mmol) was added. The resulting mixture was stirred overnight, filtered, and concentrated under reduced pressure. The residue was purified by preparative reverse-phase HPLC (water/acetonitrile gradient, containing 0.1% TFA) to give 4-{3-methyl-4′-[({[4-(trifluoromethyl)-phenyl]-aminocarbonyl)-amino]-1,1′-biphenyl-4-yl}-4-oxo-2-(2-phenyl-ethyl)butanoic acid as a white solid (19 mg, 56% yield for two steps). LC-MS RT=3.94 min, m/z 575.1); ¹H NMR (DMSO-d₆) δ 1.73-2.00 (m, 2H), 2.44 (s, 3H), 2.61-2.71 (m, 2H), 2.78-2.92 (m, 1H), 3.14 (dd, 1H), 3.33 (dd, 1H), 7.15-7.34 (m, 5H), 7.57-7.77 (m, 10H), 7.89 (d, 1H), 9.04 (s, 1H), 9.20 (s, 1H), 12.29 (br s, 1H).

### Example 10

### Preparation of 4-{3′-fluoro-4′-[(4-fluoro-3-methylbenzoyl)amino]-1,1′-biphenyl-4-yl}-2,2-dimethyl-4-oxobutanoic acid

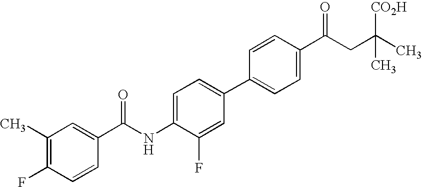

To a solution of methyl 4-(4′-amino-3′-fluoro-1,1′-biphenylyl)-2,2-dimethyl-4-oxobutanoate (40 mg, 0.12 mmol, prepared as described in US 2004/0224997) and 4-fluoro-3-methylbenzoyl chloride (25.1 mg, 0.15 mmol) in dichloromethane (2 mL) was added poly-4-vinyl pyridine (40 mg, 0.36 mmol). The resulting suspension was stirred at rt for 16 h. Solvent was then removed under reduced pressure and the mixture was dissolved in methanol (1 mL) and tetrahydrofuran (1 mL) and a 1.0 N aqueous solution of sodium hydroxide (0.5 mL, 0.5 mmol) was added. The mixture was stirred at rt for 16 h and then concentrated under reduced pressure. The residue was purified by preparative reverse-phase HPLC (water/acetonitrile gradient, containing 0.1% TFA) to afford 4-{3′-fluoro-4′-[(4-fluoro-3-methylbenzoyl)amino]-1,1′-biphenyl-4-yl}-2,2-dimethyl-4-oxobutanoic acid (14.4 mg, 26% yield over two steps). LC-MS RT=3.36 min., m/z 452.0 (MH⁺); ¹H NMR (300 MHz, DMS-d₆) δ 1.24 (s, 6H), 2.32 (s, 3H), 3.34 (s, 2H), 7.30 (t, 1H), 7.64 (dd, 1H), 7.65-7.76 (m, 2H), 7.84-7.89 (m, 3H), 7.96 (d, 1H), 8.02 (d, 2H), 10.19 (s, 1H).

### Example 11

### Preparation of 4-{4′-[(4-fluoro-3-methylbenzoyl)amino]-3′-methyl-1,1′-biphenyl-4-yl}-2,2-dimethyl-4-oxobutanoic acid

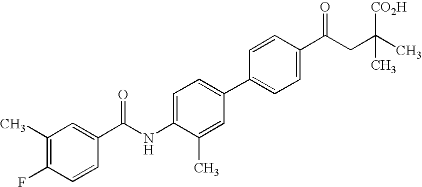

This compound was prepared in a similar manner to the procedure described in Example 10 above, using methyl 4-(4′-amino-3′-methyl-1,1′-biphenyl-4-yl)-2,2-dimethyl-4-oxobutanoate prepared as described in US 2004/0224997. LC-MS RT=3.28 min, m/z 448.1 (MH⁺); ¹H NMR (300 MHz, DMSO-d₆) δ 1.24 (s, 6H), 2.33 (s, 6H), 3.35 (s, 2H), 7.29 (t, 1H), 7.47 (d, 1H), 7.60 (dd, 1H), 7.67 (s, 1H), 7.82-7.88 (m, 3H), 7.94 (dd, 1H), 8.01 (d, 2H), 9.91 (s, 1H).

### Example 12

### Preparation of 4-[4′-({[(2-ethoxyphenyl)amino]carbonyl}amino)-3′-fluoro-1,1′-biphenyl-4-yl]-2,2-dimethyl-4-oxobutanoic acid

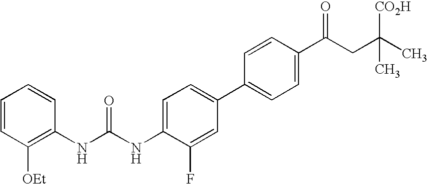

A mixture of methyl 4-(4′-amino-3′-fluoro-1,1′-biphenylyl)-2,2-dimethyl-4-oxobutanoate (40 mg, 0.12 mmol, prepared as described in US 2004/0224997), 2-ethoxyphenyl isocyanate (24 mg, 0.15 mmol) in dichloromethane (2 mL) was stirred at rt overnight. The mixture was concentrated under reduced pressure and the residue was dissolved in tetrahydrofuran (1 mL) and methanol (1 mL). Aqueous sodium hydroxide (1 N, 0.5 mL, 0.5 mmol) was then added. The mixture was then stirred at rt for 16 h and then concentrated under reduced pressure. The residue was purified by preparative reverse-phase HPLC (water/acetonitrile gradient, containing 0.1% TFA) to afford 4-[4′-({[(2-ethoxyphenyl)amino]carbonyl}amino)-3′-fluoro-1,1′-biphenyl-4-yl]-2,2-dimethyl-4-oxobutanoic acid (17.6 mg, 30% yield over two steps). LC-MS RT=3.42 min., m/z 479.5 (MH⁺); ¹H NMR (300 MHz, DMSO-d₆) δ 1.24 (s, 6H), 1.43 (t, 3H), 3.34 (s, 2H), 4.15 (q, 2H), 6.90 (t, 1H), 7.02 (d, 1H), 7.57 (dd, 1H), 7.69 (dd, 1H), 7.83 (d, 2H), 8.01 (d, 2H), 8.11 (dd, 1H), 8.30 (t, 1H), 8.65 (s, 1H), 9.44 (s, 1H).

### Example 13

### Preparation of 4-[4′-({[(2-ethoxyphenyl)amino]-carbonyl}-amino)-3′-methyl-1,1′-biphenyl-4-yl]-2,2-dimethyl-4-oxobutanoic acid

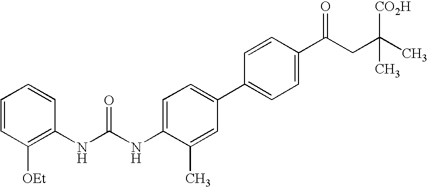

This compound was prepared in a similar manner to the procedure described in Example 12 above, using methyl 4-(4′-amino-3′-methyl-1,1′-biphenyl-4-yl)-2,2-dimethyl-4-oxobutanoate prepared as described in US 2004/0224997. LC-MS RT=3.37 min., m/z 475.0 (MH⁺); ¹H NMR (300 MHz, DMSO-d₆) δ 1.24 (s, 6H), 1.41 (t, 3H), 2.35 (s, 3H), 3.34 (s, 2H), 4.15 (q, 2H), 6.86-6.94 (m, 2H), 7.00 (d, 2H), 7.54 (dd, 1H), 7.61 (s, 1H), 7.78 (d, 2H), 7.92 (d, 1H), 8.00 (d, 2H), 8.08 (dd, 1H), 8.50 (s, 1H), 8.67 (s, 1H).

### Example 14

### Preparation of 4-[4′-({[(2-ethoxyphenyl)amino]carbonyl}amino)-3′-methoxy-1,1′-biphenyl-4-yl]-2,2-dimethyl-4-oxobutanoic acid

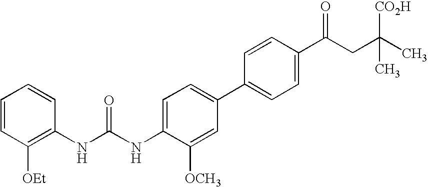

A mixture of methyl 4-(4′-amino-3′-methoxy-1,1′-biphenylyl)-2,2-dimethyl-4-oxobutanoate (50 mg, 0.15 mmol, prepared as described in US 2004/0224997), 2-ethoxyphenyl isocyanate (29 mg, 0.18 mmol) in dichloromethane (2 mL) was stirred at rt overnight. The mixture was concentrated under reduced pressure and the residue was dissolved in tetrahydrofuran (1 mL) and methanol (1 mL). Aqueous sodium hydroxide (1 N, 0.5 mL, 0.5 mmol) was then added. The mixture was then stirred at rt for 16 h and then concentrated under reduced pressure. The residue was purified by preparative reverse-phase HPLC (water/acetonitrile gradient, containing 0.1% TFA) to afford 4-[4′-({[(2-ethoxyphenyl)amino]carbonyl}amino)-3′-methoxy-1,1′-biphenyl-4-yl]-2,2-dimethyl-4-oxobutanoic acid (25.8 mg, 36% yield over two steps). LC-MS RT=3.46 min., m/z 491.0 (1); ¹H NMR (300 MHz, DMSO-d₆) δ 1.24 (s, 6H), 1.43 (t, 3H), 3.34 (s, 2H), 4.15 (q, 2H), 6.90 (t, 1H), 7.02 (d, 1H), 7.57 (dd, 1H), 7.69 (dd, 1H), 7.83 (d, 2H), 8.01 (d, 2H), 8.11 (dd, 1H), 8.30 (t, 1H), 8.65 (s, 1H), 9.44 (s, 1H).

### Example 15

### Preparation of 4-oxo-4-{4-[6-(Pentanoylamino)-3-pyridinyl]phenyl}-2-(2-phenylethyl)butanoic acid (trifluoroacetate salt)

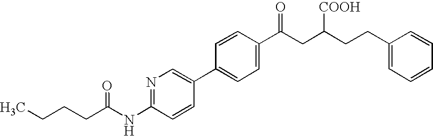

### Step 1. Preparation of ethyl 4-[4-(6-amino-3-pyridinyl]4-oxo-2-(2-phenylethyl)-butanoate

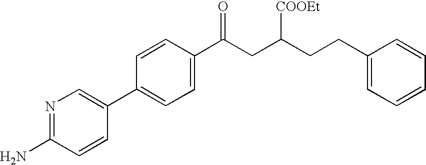

A mixture of ethyl 4-(4-bromophenyl)-4-oxo-2-(2-phenylethyl)butanoate (2.0 g, 5.2 mmol), bis(pinacolato)diboron (1.44 g, 5.69 mmol), and potassium acetate (1.51 g, 15.4 mmol) in dioxane (100 mL) was degassed by a flow of argon for 20 min. Then, [1,1′-bis(diphenylphosphino)-ferrocene]dichloro palladium(II) (1:1 complex with dichloro-methane, 0.21 g, 0.26 mmol) was added, and this reaction mixture was heated at 80° C. for 3 h. The mixture was cooled to rt, then filtered through a pad of celite and extracted with ethyl acetate. The combined organic layers were dried over anhydrous sodium sulfate and concentrated under reduced pressure to afford ethyl 4-oxo-2-(2-phenylethyl)-4-[4-(4,4,5,5-tetramethyl-1,3,2-dioxaborolan-2-yl)phenyl]butanoate (3 g) as a black oil. A mixture of 0.5 g (estd. 0.856 mmol) of this intermediate, 2-amino-5-bromopyridine (297 mg, 1.72 mmol), and sodium bicarbonate (963 mg, 11.46 mmol) in toluene (50 mL) and water (9.3 mL) was degassed by a flow of argon for 20 min. Then, [1,1′-bis(diphenylphosphino)-ferrocene]dichloro palladium(II) (1:1 complex with dichloromethane, 94 mg, 0.115 mmol) was added, and this reaction mixture was heated at 85° C. for 3 h. The mixture was cooled to rt, then filtered through a pad of celite and extracted with ethyl acetate. The combined organic layers were dried over anhydrous sodium sulfate and concentrated under reduced pressure to afford ethyl 4-[4-(amino-3-pyridinyl)phenyl]-4-oxo-2-(2-phenylethyl)butanoate as a light yellow oil (93 mg, 27% overall for two steps). LC-MS RT=2.80 min (method 2), m/z 403 (MH; ¹H NMR (300 MHz, CDCl₃) δ 8.22 (s, 1H), 7.90 (d, 2H), 7.55 (d, 1H), 7.50 (d, 2H), 7.20-7.10 (m, 5H), 6.60 (d, 1H), 4.80 (br s, 2H), 4.10 (q, 2H), 3.50 (m, 1H), 3.00 (m, 2H), 2.60 (m, 2H), 2.00 (m, 2H), 1.20 (t, 3H).

### Step 2. Preparation of 4-oxo-4-{4-[6-(pentanoylamino)-3-pyridinyl]phenyl}-2-(2-phenylethyl)-butanoic acid (trifluoroacetate salt)

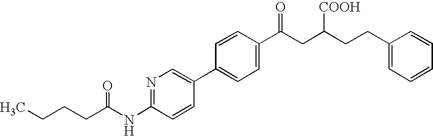

To a solution of ethyl 4-[4-(6-amino-3-pyridinyl)phenyl]-4-oxo-2-(2-phenylethyl)butanoate (15 mg, 0.037 mmol) in dichloroethane (1 mL), valeryl chloride (6.7 mg, 0.056 mmol) and PS-DIEA (20 mg, 5.7 mmol) was added, and the resulting suspension was mixed by orbital shaking at rt overnight. The reaction mixture was filtered and then dried under reduced pressure (GeneVac evaporator). The solid residue was re-dissolved in 1:1 tetrahydrofuran/methanol (1 mL), 1 N aqueous sodium hydroxide (0.15 mL) was added, and the mixture was shaken overnight at rt. A solution of 2 N aqueous hydrochloric acid (0.1 mL) was added, and the mixture was dried under reduced pressure (GeneVac evaporator). The residue was purified by preparative reverse-phase HPLC (water/acetonitrile gradient, containing 0.1% TFA) to afford 4-oxo-{4-[6-(pentanoylamino)-3-pyridinyl]phenyl}-2-(2-phenylethyl)butanoic acid (trifluoroacetate salt) as a white solid (6.4 mg, 37.6%). LC-MS RT=3.00 min (method 2), m/z 459.2 (MH⁺); ¹H NMR (300 MHz, CDCl₃) δ 12.20 (s, 1H), 10.50 (s, 1H), 8.70 (s, 1H), 8.20 (m, 2H), 8.05 (d, 2H), 7.90 (d, 2H), 7.1-7.3 (m, 5H), 3.55 (dd, 1H), 3.22 (m, 1H), 2.90 (m, 1H), 2.65 (m, 2H), 2.40 (t, 2H), 1.80 (m, 2H), 1.60 (m, 2H), 1.37 (m, 2H), 0.94 (t, 3H).

### Example 16

### Preparation of 4-{4-[5-({[(2-chlorophenyl)amino]carbonyl}amino)-2-pyridinyl]phenyl}-4-oxo-2-(2-phenylethyl)butanoic acid (trifluoroacetate salt)

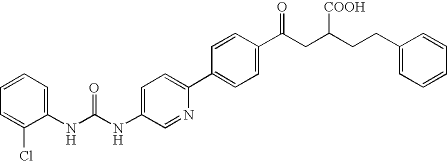

### Step 1. Preparation of methyl 4-[4-(5-amino-2-pyridinyl)phenyl]-4-oxo-2-(2 phenylethyl)butanoate

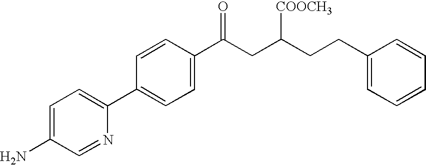

The procedure was similar to that described for the syntheses of ethyl 4-[(6-amino-3-pyridinyl)phenyl]-4-oxo-2-(2-phenylethyl)butanoate (Example 15), but using 3-amino-6-bromopyridine in place of 2-amino-5-bromopyridine. The product was obtained as a yellow solid (26% yield). ¹H NMR (300 MHz, CDCl₃) δ 8.05 (d, 3H), 7.90 (d, 2H), 7.75 (d, 1H), 7.25 (m, 2H), 7.20 (m, 3H), 6.95 (d, 1H), 5.70 (s, 2H), 3.55 (s, 3H), 3.40 (m, 1H), 3.30 (m, 1H), 2.95 (m, 1H), 2.65 (t, 2H), 1.90 (m, 2H); LC-MS RT=2.53 min (method 2), m/z 403 (MH⁺).

Step 2. Preparation of 4-{4-[5-({[(2-chlorophenyl)amino]carbonyl}amino)-2-pyridinyl]phenyl}-4-oxo-2-(2-phenylethyl)butanoic acid (trifluoroacetate salt)

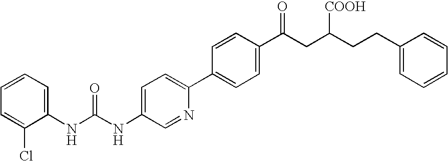

The procedure (urea formation, followed by ester hydrolysis) was similar to that described above in Example 5. The product was obtained as a white solid (63% yield). ¹H NMR (300 MHz, DMSO-d₆) δ 9.80 (s, 1H), 8.70 (s, 1H), 8.50 (s, 1H), 8.20 (m, 4H), 8.00 (m, 3H), 7.45 (d, 1H), 7.25 (m, 3H), 7.15 (m, 3H), 7.00 (m, 1H), 3.55 (m, 1H), 3.20 (m, 1H), 2.95 (m, 1H), 2.65 (m, 2H), 1.90 (m, 2H); LC-MS RT=3.29 min (method 2), m/z 528.2 (MH⁺).

### Example 17

### Preparation of 4-[4′-({[(2,4-difluorophenyl)amino]carbonyl}amino)-2′-methyl-1,1′-biphenyl-4-yl]-4-oxo-2-(2-phenylethyl)butanoic acid

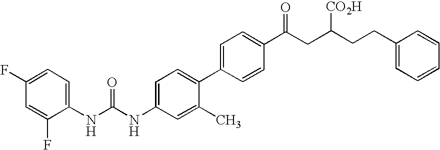

### Step 1. Preparation of ethyl 4-(4′-amino-2′-methyl-1,1′-biphenyl-4-oxo-2-(2-phenylethyl)butanoate

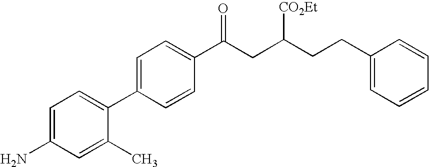

The procedure was similar to that described for the synthesis of ethyl 4-[4-(6-amino-3-pyridinyl)phenyl]-4-oxo-2-(2-phenylethyl)butanoate (Example 15), but using 3-methyl-4-bromoaniline in place of 2-amino-5-bromopyridine. The product was obtained as a yellow solid (34% yield). ¹H NMR (300 MHz, CDCl₃) δ 7.90 (d, 2H), 7.30 (d, 2H), 7.20 (m, 2H), 7.10 (m, 3H), 6.95 (d, 1H), 6.50 (m, 2H), 4.00 (, 2H), 3.70 (broad s, 2H), 3.40 (m, 1H), 3.00 (m, 2H), 2.60 (m, 2H), 2.15 (s, 3H), 1.95-1.85 (m, 2H), 1.20 (t, 3H) LC-MS RT=2.89 min (method 2), m/z 416.2 (MH⁺).

### Step 2. Preparation of 4-[4′-({[(2,4-difluorophenyl)amino]carbonyl}amino)-2′-methyl-1,1′-biphenyl-4-yl]-4-oxo-2-(2-phenylethyl)butanoic acid

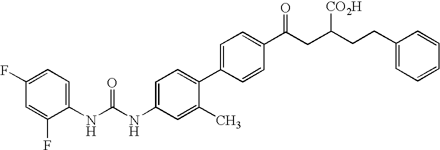

The procedure (urea formation, followed by ester hydrolysis) was similar to that described above in Example 5. The product was obtained as a white solid (62% yield). ¹H NMR (300 MHz, DMSO-d₆) δ 12.2 (s, 1H), 9.05 (s, 1H), 8.50 (s, 1H), 8.00 (m, 3H), 7.50-7.00 (m, 12H), 3.40 (m, 1H), 3.20 (m, 1H), 2.80 (m, 1H), 2.60 (m, 2H), 2.20 (s, 3H), 1.90-1.80 (m, 2H); LC-MS RT=4.26 min (method 2), m/z 543.3 (MH⁺).

### Example 18

### Preparation of 2-benzyl-4-{4-[6-({[(3,4-dimethylphenyl)amino]carbonyl}amino)-2-methyl-3-pyridinyl]phenyl}-4-oxobutanoic acid (trifluoroacetate salt)

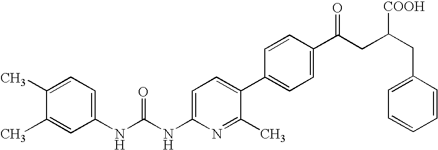

### Step 1 Preparation of ethyl 4-[4-(6-amino-2-methyl-3-pyridinyl)phenyl]-2-benzyl-4-oxobutanoate

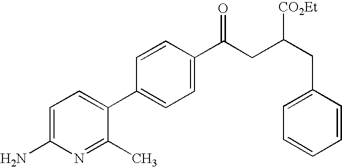

The procedure was similar to that described for the synthesis of ethyl 4-[4-(6-amino-3-pyridinyl)phenyl]-4-oxo-2-(2-phenylethyl)butanoate (Example 15), but using 5-bromo-6-methyl-2-pyridinamine in place of 2-amino-5-bromopyridine. The product was obtained as a yellow solid (66% yield); LC-MS RT=2.87 min (method 2), m/z 390.2 (MH⁺).

### Step 2 Preparation of 2-benzyl-4-{4-[6-({[(3,4-dimethylphenyl)amino]carbonyl}amino)-2-methyl-3-pyridinyl]phenyl}-4-oxobutanoic acid (trifluoroacetate salt)

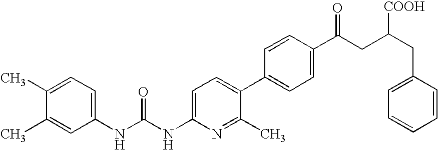

To a solution of ethyl 4-[4-(6-amino-2-methyl-3-pyridinyl)phenyl]-2-benzyl-4-oxobutanoate (30 mg, 0.077 mmol) in DCE (1 mL), 3,4-dimethylphenyl isocyanate (17.6 mg, 0.12 mmol) was added, and the mixture was stirred at rt overnight. The solvent was removed under reduced pressure (GeneVac evaporator) and the solid was redissolved in DMF (3 mL). A solution of 1 N NaOH (0.1 mL, 0.11 mmol) was added, and the mixture was stirred at rt overnight. A solution of 1 N HCl (0.1 mL, 0.11 mmol) and methanol (5 mL) were added, and the product was isolated and purified by preparative reverse-phase HPLC (water/acetonitrile gradient, containing 0.1% TFA) to give 2-benzyl-{4-[6-({[(3,4-dimethylphenyl)amino]carbonyl}amino)-2-methyl-3-pyridinyl]phenyl}-4-oxo-butanoic acid (trifluoroacetate salt) (62% yield). ¹H NMR (300 MHz, DMSO-d₆) δ 10.5 (bs, 1H), 9.60 (s, 1H), 8.00 (d, 2H), 7.66 (d, 1H), 7.50 (d, 2H), 7.30 (d, 1H), 7.20 (m, 7H), 7.00 (d, 2H), 3.40 (q, 1H), 3.20 (m, 1H), 3.00 (m, 2H), 2.90 (m, 1H), 2.50 (s, 3H), 2.25 (s, 3H), 2.20 (s, 3H); LC-MS RT=3.42 min (method 2), m/z 522.3 (MH⁺).

### Example 19

### Preparation of 4-oxo-2-(2-phenylethyl)-4-(4-{2-[({[4-(trifluoromethyl)phenyl]amino}-carbonyl)amino]-5-pyrimidinyl}phenyl)butanoic acid (trifluoroacetate salt)

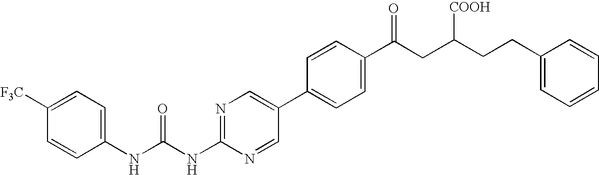

### Step 1. Preparation of methyl 4-[4-(2-amino-5-pyrimidinyl)phenyl]oxo-2-(2-phenylethyl)-butanoate

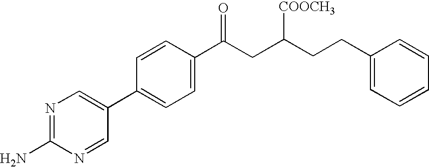

The procedure was similar to that described for the synthesis of ethyl 4-[4-(6-amino-3-pyridinyl)phenyl]-4-oxo-2-(2-phenylethyl)butanoate (Example 15), but using 5-bromo-2-pyrimidinamine in place of 2-amino-5-bromopyridine. The product was obtained as a brown solid (79% yield); LC-MS RT=2.87 min (method 2), m/z 390.2 (MH⁺).

### Step 2. Preparation of 4-oxo-2-(2-phenylethyl)-4-(4-{2-[({[-4-(trifluoromethyl)phenyl]-amino}carbonyl)amino]-5-pyrimidinyl}phenyl)butanoic acid (trifluoroacetate salt)

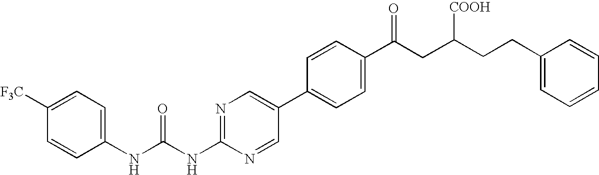

To a solution of methyl 4-[4-(2-amino-5-pyrimidinyl)phenyl]-4-oxo-2-(2-phenylethyl) butanoate (30 mg, 0.077 mmol) in DCE (1 mL), 4-trifluoromethylphenyl isocyanate (21.6 mg, 0.12 mmol) was added, and the mixture was stirred at rt overnight. The solvent was removed under reduced pressure (GeneVac evaporator), and the solid was redissolved in DMP (3 mL). A solution of 1 N NaOH (0.1 mL, 0.1 mmol) was then added, and the mixture was again stirred at rt overnight. A solution of 1 N HCl (0.1 mL, 0.1 mmol) was added to the reaction mixture, and the product was isolated and purified by preparative reverse-phase HPLC (water/acetonitrile gradient, containing 0.1% TFA) to give 4-oxo-2-(2-phenylethyl)-4-(4-{2-[({[4-(trifluoromethyl)phenyl]amino}carbonyl)amino]-5-pyrimidinyl}phenyl)butanoic acid (trifluoroacetate salt) as a white solid (76% yield). ¹H NMR (300 MHz, DMSO) δ 8.10 (d, 2H), 7.95 (d, 2H), 7.80 (d, 2H), 7.65 (m, 3H), 7.20 (m, 6H), 3.50 (m, 1H), 3.20 (m, 1H), 2.90 (m, 1H), 2.60 (m, 2H), 1.90 (m, 2H); LC-MS RT=3.71 min (method 1), m/z 563.0 (MH⁺).

### Example 20

### Preparation of 4-oxo-4-[4′-(pentanoylamino)-1,1′-biphenyl-4-yl]butanoic acid

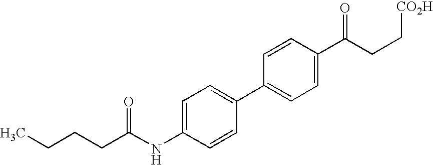

### Step 1. Preparation of diethyl 2-[2-($bromophenyl)-2-oxoethyl]malonate

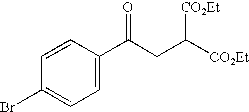

To a 250 mL 3-neck round-bottom flask fitted with an argon inlet, a septum, and an addition funnel was added sodium hydride (60% in mineral oil, 1.75 g, 43.7 mmol) followed by tetrahydrofuran (25 mL). The suspension was then cooled to 0° C., and diethyl malonate (7.0 g, 43.7 mmol) in tetrahydrofuran (20 mL) was added dropwise over 20 min. The cooling bath was then removed and the reaction mixture was allowed to warm to rt over 45 min. A solution of 2-bromo-1-(4-bromophenyl)ethanone (8.08 g, 43.7 mmol) in tetrahydrofuran (35 mL) was added rapidly, giving a yellow mixture that was stirred at rt for 16 h, and then poured into 200 mL of 1.0 N aqueous hydrochloric acid. The mixture was stirred for 10 min and extracted with ethyl acetate twice. The combined extracts were dried over anhydrous sodium sulfate and concentrated under reduced pressure to afford diethyl 2-[2-(4-bromophenyl)-2-oxoethyl]malonate (10.2 g, 66%) which was used in the next step without further purification. GC-MS RT=3.89 min, m/z 357 (MH⁺); ¹H NMR (300 MHz, CDCl₃) δ 1.27 (t, 6H), 3.55 (d, 2H), 4.02 (t, 1H), 4.15-4.27 (m, 4H), 7.59 (d, 2H), 7.82 (d, 2H).

### Step 2. Preparation of diethyl 2-[2-(4′-nitro-1,1′-biphenyl-4-yl)-2-oxoethyl]malonate

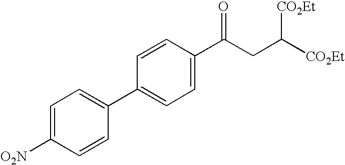

A solution of diethyl 2-[2-(4-bromophenyl)-2-oxoethyl]malonate (8.20 g, 22.9 mmol) and 4-nitrophenyl boronic acid (4.20 g, 25.2 mmol) in dry toluene (200 mL) and dioxane (50 mL) was degassed for 30 min. Saturated aqueous sodium carbonate (60 mL) and [1,1′-bis-(diphenylphosphino)-ferrocene]dichloro palladium(II) (1:1 complex with dichloromethane, 934 mg, 1.14 mmol) were added as degassing was continued. The resulting mixture was then heated at 85° C. for 16 h before it was cooled to rt. Water was added and the layers were separated. The aqueous layer was extracted with twice with ethyl acetate. The combined organic extracts were then dried over sodium sulfate, and concentrated under reduced pressure. The residue was purified by silica gel flash chromatography (Biotage flash 75, 5:1 ethyl acetate:hexane) to afford diethyl 2-[2-(4′-nitro-1,1′-biphenylyl)-2-oxoethyl]malonate (4.8 g, 53%). LC-MS RT=3.41 min, m/z 400.1); ¹H NMR (300 MHz, CDCl₃) δ 1.30 (t, 6H), 3.65 (d, 2H), 4.08 (t, 1H), 4.22-4.29 (m, 4H), 7.70-7.79 (m, 4H), 8.09 (d, 2H), 8.32 (d, 2H).

### Step 3. Preparation of diethyl 2-[2-(4′-amino-1,1′-biphenyl-4-yl)-2-oxoethyl]malonate

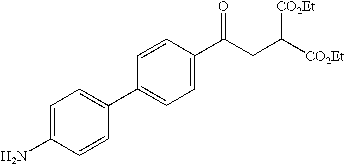

To a solution of diethyl 2-[2-(4′-nitro-1,1′-biphenyl-4-yl)-2-oxoethyl]malonate (3.50 g, 8.77 mmol) in 85:15 ethanol/water (115 mL) was added iron powder (64.9 g) followed by 2 N aqueous hydrochloric acid (4.38 mL). The resulting mixture was refluxed for 2.5 h, then filtered through a pad of celite. The filtrate was extracted with ethyl acetate, and the combined organic layers was then dried over sodium sulfate and concentrated under reduced pressure to afford diethyl 2-[2-(4′-amino-1,1′-biphenyl-4-yl)-2-oxoethyl]malonate (3.18 g, 98%). LC-MS RT=3.23 min, m/z 370.3 (MH⁺); ¹H NMR (300 MHz, CDCl₃) δ 1.20 (t, 6H), 3.56 (d, 2H), 3.8 (br s, 2H), 4.02 (t, 1H), 4.18 (q, 4H), 6.71 (d, 2H), 7.39 (d, 2H), 7.54 (d, 2H), 7.94 (d, 2H).

### Step 4. Preparation of diethyl 2-{2-oxo-2-[4′-(pentanoylamino)-1,1′-biphenyl-4-yl]ethyl}-malonate

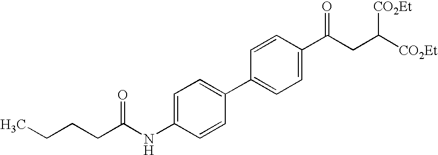

To a solution of diethyl 2-[2-(4′-amino-1,1′-biphenyl-4-yl)-2-oxoethyl]malonate (3.17 g, 8.58 mmol) and valeryl chloride (1.24 g, 10.3 mmol) in dichloromethane (55 mL) was added poly-4-vinyl pyridine (2.8 g, 27.7 mmol). The resulting suspension was stirred at rt for 3 h and then filtered. The filtrate was washed with water, dried over sodium sulfate, and concentrated under reduced pressure to afford diethyl 2-{2-oxo-2-[4′-(pentanoylamino)-1,1′-biphenyl-4-yl]ethyl}malonate (3.6 g, 93%). LC-MS RT=3.99 min, m/z 454.3 (MH⁺); ¹H NMR (300 MHz, CDCl₃) δ 0.89 (t, 3H), 1.22 (t, 6H), 1.32-1.37 (m, 2H), 1.64-1.69 (m, 2H), 2.32 (t, 2H), 3.56 (d, 2H), 4.00 (t, 1H), 4.15-4.21 (m, 4H), 7.14 (s, 1H), 7.50-7.60 (m, 6H), 7.95 (d, 2H).

### Step 5. Preparation of 2-{2-oxo-2-[4′-(pentanoylamino)-1,1′-biphenyl-4-yl]ethyl}-malonic acid

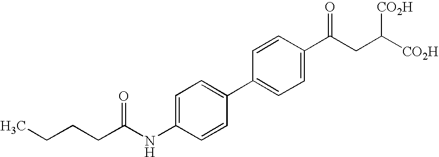

To a flask containing diethyl 2-{2-oxo-2-[4′-(pentanoylamino)-1,1′-biphenyl-4-yl]ethyl}malonate (1.60 g, 3.53 mmol) was added ethanol (25 mL) followed by 1.0 N aqueous sodium hydroxide solution (17.6 mL), and the resulting mixture was stirred at rt for 16 h. The suspension was then concentrated under reduced pressure to remove ethanol, and then the aqueous layer was acidified with 1.0 N aqueous hydrochloric acid and stirred for 10 min. The mixture was then extracted twice with ethyl acetate, and the combined organic layers were dried over anhydrous sodium sulfate and concentrated under reduced pressure to afford 2-(2-oxo-2-[4′-(pentanoylamino)-1,1′-biphenyl-4-yl]ethyl}malonic acid (1.34 g, 96%). LC-MS RT=3.29 min, m/z 398.5 (MH⁺); ¹H NMR (300 M, DMSO-d₆) δ 0.90 (t, 3H), 1.29-1.38 (m, 2H), 1.53-1.63 (m, 2H), 2.32 (t, 2H), 3.52 (d, 2H), 3.77 (t, 1H), 7.69 (s, 4H), 7.78 (d, 2H), 8.02 (d, 2H), 10.03 (s, 1H).

### Step 6. Preparation of 4-oxo-4-[4′-(pentanoylamino)-1,1′-biphenyl-4-yl]butanoic acid

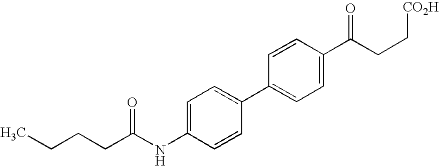

A solution of 2-{2-oxo-2-[4′-(pentanoylamino)-1,1′-biphenyl-4-yl]-ethyl}malonic acid (1.33 g, 335 mmol) in 1,4-dioxane (60 mL) was heated to reflux for 16 h. The mixture was cooled to rt, and was then concentrated under reduced pressure to afford 4-oxo-4-[4-(pentanoylamino)-1,1′-biphenyl-4-yl]butanoic acid (1.15 g, 98%). LC-MS RT=2.73 min, m/z 354.2); ¹H NMR (300 MHz, DMSO-d₆) δ 0.88 (t, 3H), 1.27-1.35 (m, 2H), 1.54-1.59 (m, 2H), 2.31 (t, 2H). 2.57 (t, 2H), 3.25 (t, 2H), 7.70 (s, 4H), 7.80 (d, 2H), 8.00 (d, 2H), 10.01 (s, 1H), 12.20 (s, 1H).

### Example 21

### Preparation of 2-[2-(4-fluorophenyl)ethyl]-4-oxo-4-[4′-(pentanoylamino)-1,1′-biphenyl-4-yl]butanoic acid

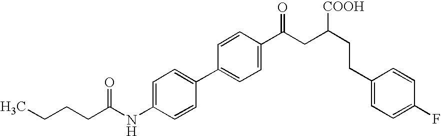

### Step 1. Preparation of 1-(2-iodoethyl)-4-fluorobenzene

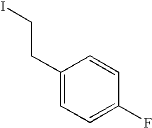

To a solution of 1-(2-chloroethyl)-4-fluorobenzene (400 mg, 2.52 mmol) in acetone (20 mL) was added sodium iodide (3.78 g, 25.2 mmol) and the resulting suspension was heated to reflux for 16 h. The mixture was filtered, and the filtrate was concentrated under reduced pressure. The residue was dissolved in dichloromethane and the organic layer was washed with water. The organic layer was dried over anhydrous sodium sulfate and concentrated under reduced pressure to give 1-(2-iodoethyl)-4-fluorobenzene (610 mg, 97%). GC-MS m/z 250 (M⁺); RT=5.53 min.; ¹H NMR (300 MHz, CDCl₃) δ 3.14 (t, 2H), 3.29-3.35 (m, 2H), 6.97-7.04 (m, 2H), 7.13-7.18 (m, 2H).

### Step 2. Preparation of 2-[2-(4-fluorophenyl)ethyl]-4-oxo-4-[4-(pentanoylamino)-1,1′-biphenyl-4-yl]butanoic acid

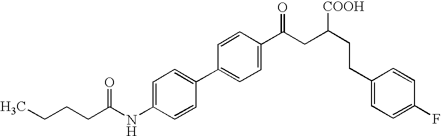

To a solution of diethyl 2-{2-oxo-2-[4′-(pentanoylamino)-1,1′-biphenylyl-4-yl]ethyl}malonate (Example 15) (100 mg, 0.220 mmol) in tetrahydrofuran (1.0 mL) was added sodium hydride (13.2 mg, 0.330 mmol, 60% disperion in mineral oil) and the resulting solution was stirred at rt for 30 min. A solution of 1-(2-iodoethyl)-4-fluorobenzene (110 mg, 0.440 mmol) in tetrahydrofuran (1.0 mL) was added and the resulting solution was heated at 60° C. for 16 h. The mixture was concentrated under reduced pressure and the residue was dissolved in 2.0% ethanolic potassium hydroxide (3.0 mL). The resulting mixture was stirred at rt for 16 h and was then concentrated under reduced pressure. The aqueous layer was acidified with 1.0 N aqueous hydrochloric acid and the mixture was extracted twice with ethyl acetate. The combined organic layer was dried over anhydrous sodium sulfate and concentrated under reduced pressure. The residue was dissolved in 1,4-dioxane (2 mL) and heated at 100° C. for 16 h before it was cooled to rt. The resulting mixture was concentrated under reduced pressure and the residue was purified by preparative reverse-phase HPLC (water/acetonitrile gradient, containing 0.1% TFA) to afford 2-[2-(4-fluorophenyl)ethyl]-4-oxo-4-[4′-(pentanoylamino)-1,1′-biphenyl-4-yl]butanoic acid (3.5 mg, 4%). LC-MS RT=3.12 min., m/z 476 (MH⁺); ¹H NMR (300 MHz, DMSO-d₆) δ 0.91 (t, 3H), 1.27-1.35 (m, 2H), 1.52-1.59 (m, 2H), 1.80-1.88 (m, 2H), 2.31 (t, 2H), 2.64 (t, 2H), 2.81-2.87 (m, 1H), 3.15 (dd, 1H), 3.41-3.49 (m, 1H), 7.06 (t, 2H), 7.21-7.26 (m, 2H), 7.70 (s, 4H), 7.77 (d, 2H). 8.00 (d, 2H).

### Example 22

### Preparation of 2-ethyl-4-oxo-4-[4′-(pentanoylamino)-1,1′-biphenyl-4-yl]butanoic acid

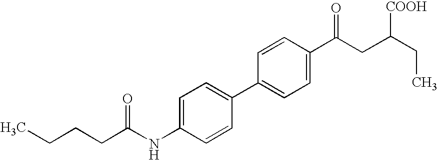

To a solution of diethyl 2-{2-oxo-2-[4′-(pentanoylamino)-1,1′-biphenyl-4-yl]ethyl}malonate (Example 15) (100 mg, 0.220 mmol) in tetrahydrofuran (1.0 mL) was added sodium hydride (11 mg, 0.26 mmol, 60% dispersion in mineral oil) and the resulting solution was stirred at rt for 30 min. Ethyl iodide (49 mg, 0.31 mmol) was then added in tetrahydrofuran (1.0 mL) and the resulting solution was heated at 60° C. for 16 h. The mixture was concentrated under reduced pressure and the residue was dissolved in ethanol (1.5 mL). Aqueous sodium hydroxide solution (1.0 N, 1.1 mL) was added and the resulting mixture was stirred at rt for 16 h. The suspension was concentrated under reduced pressure and the aqueous layer was acidified with 1.0 N aqueous hydrochloric acid. The mixture was then extracted twice with ethyl acetate, and the combined organic layers were dried over anhydrous sodium sulfate and concentrated under reduced pressure. The mixture was then dissolved in 1,4-dioxane (2 mL) and heated at 100° C. for 16 h before it was cooled to rt. The mixture was concentrated under reduced pressure and the residue was purified by preparative reverse-phase HPLC (water/acetonitrile gradient, containing 0.1% TFA) to afford 2-ethyl-4-oxo-4-[4′-(pentanoylamino)-1,1′-biphenyl-4-yl]butanoic acid (3.7 mg, 5%). LC-MS RT=2.99 min., m/z/z 382.1 (MH⁺); ¹H NMR (300 MHz, DMSO-d₆) δ 0.91-0.99 (m, 6H), 1.21-1.37 (m, 2H), 1.51-1.64 (m, 4H), 3.32 (t, 2H), 2.70-2.79 (m, 2H), 3.10 (dd, 1H), 3.33-3.43 (m, 1H), 7.69 (s, 4H), 7.77 (d, 2H), 8.00 (d, 2H) 10.01 (s, 1H).

### Example 23

### Preparation of ethyl 2-[2-(4′-{[(4-chlorophenyl)acetyl]amino}-1,1′-biphenyl-4-yl)-2-oxoethyl]pentanoate

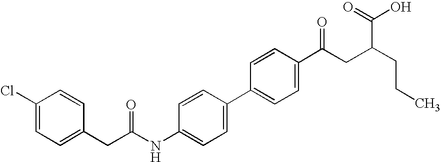

To a standard amber 4 mL vial was added methyl 2-[2-(4′-amino-1,1-biphenylyl)-2-oxoethyl]pentanoate (35 mg, 0.10 mmol, prepared as described in US 2004/0224997) dissolved in dichloromethane (1 mL), followed by addition of poly-4-vinyl pyridine (34 mg, 0.31 mmol) and a solution of 4-chlorophenylacetyl chloride (17.6 mg, 0.093 mmol) in dichloromethane (1 mL). The resulting suspension was stirred at rt for 16 h, then filtered. The filtrate was concentrated under reduced pressure and the mixture was dissolved in methanol (1 mL) and tetrahydrofuran (1 mL). An aqueous sodium hydroxide solution (1.0 N, 0.31 mL) was added, and the reaction mixture was stirred at rt for 16 h and then concentrated under reduced pressure. The residue was purified by preparative reverse-phase HPLC (water/acetonitrile gradient, containing 0.1% trifluoroacetic acid) to afford 2-[2-(4′-{[(4-chlorophenyl)acetyl]amino}-1,1′-biphenyl-4-yl)-2-oxoethyl]pentanoic acid (8 mg, 17%). LC-MS RT=4.01 min., m/z 464.2 (MH⁺); ¹H NMR (300 MHz, DMSO-d6) δ 0.86 (t, 3H), 1.25-1.40 (m, 2H), 1.40-1.64 (m, 2H), 2.75-2.85 (m, 1H), 3.07 (dd, 1H), 3.2-3.45 (m, 1H), 3.65 (s, 2H), 7.35 (d, 4H), 7.70 (s, 4H), 7.77 (d, 2H), 8.0 (d, 2H), 10.32 s, 1H), 12.08 (br s, 1H).

### Example 24

### Preparation of 2-{2-[4′-({[(2-chlorophenyl)amino]carbonyl}amino)-1,1′-biphenyl-4-yl]-2-oxoethyl}pentanoic acid

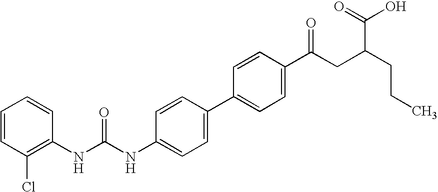

To a standard amber 4 mL vial was added methyl 2-[2-(4′-amino-1,1′-biphenylyl)-2-oxoethyl]pentanoate (35 mg, 0.10 mmol, prepared as described in US 2004/0224997), 2-chlorophenylisocyanate (24 mg, 0.15 mmol), and dichloromethane (2 mL) and the resulting solution was stirred for 16 h. The reaction mixture was filtered, the filtrate concentrated under reduced pressure, and the mixture dissolved in methanol (1 mL) and tetrahydrofuran (1 mL), followed by addition of 1.0 N aqueous sodium hydroxide solution (0.31 mL). The reaction mixture was stired at rt for 16 h and then concentrated under reduced pressure. The residue was purified by preparative reverse-phase HPLC (water/acetonitrile gradient, containing 0.1% trifluoroacetic acid) to afford 2-{2-[4′-({[(2-chlorophenyl)amino]carbonyl}amino)-1,1′-biphenyl-4-yl]-2-oxoethyl}pentanoic acid (15 mg, 32%). LC-MS RT=3.43 min., m/z 465.2 (MH⁺); ¹H NMR (300 MHz, DMSO-d₆) δ 0.9 (t, 3H), 1.25-1.50 (m, 2H), 1.50-1.66 (m, 2H), 2.77-2.92 (m, 1H), 3.10 (dd, 1H), 3.22-3.47 (m, 1H), 7.00-7.08 (m, 1H), 7.25-7.35 (m, 1H), 7.46 (d, 1H), 7.60 (d, 2H), 7.68-7.85 (2d, 4H), 8.02 (d, 2H), 8.16 (d, 1H), 8.37 (s, 1H), 9.6 (s, 1H), 12.1 (br s, 1H).

### Example 25

### Preparation of 4-(4′-{[(4-chlorophenyl)acetyl]amino}-1,1′-biphenyl-4-yl)-2-(2-methoxyethyl)-4-oxobutanoic acid

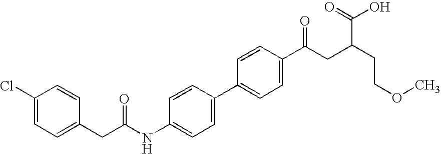

To a standard amber 4 mL vial was added ethyl 4-(4′-amino-1,1′-biphenyl-4-yl)-2-(2-methoxyethyl)-4-oxobutanoate (35 mg, 0.10 mmol, prepared as described in US 2004/0224997) dissolved in 1 mL dichloromethane, followed by addition of poly-4-vinyl pyridine (33 mg, 0.30 mmol) and a dichloromethane solution (1 mL) of 4-chlorophenylacetyl chloride (28.4 mg, 0.15 mmol). The resulting suspension was stirred at rt for 16 h, then filtered. The filtrate was concentrated under reduced pressure and the residue was dissolved in methanol (1 mL) and tetrahydrofuran (1 mL). An aqueous sodium hydroxide solution (1 N, 0.31 mL) was added and the reaction mixture was stirred at rt for 16 h and then concentrated under reduced pressure. The residue was purified by preparative reverse-phase HPLC (water/acetonitrile gradient, containing 0.1% trifluoroacetic acid) to afford 4-(4′-{[(4-chlorophenyl)acetyl]amino}-1,1′-biphenyl-4-yl)-2-(2-methoxyethyl)-4-oxobutanoic acid (20 mg, 41%). LC-MS RT=3.06 min, m/z 480.0 (MH⁺); ¹H NMR (300 MHz, DMSO-d6) δ: 1.66-1.95 (overlapping m, 2H), 2.83-2.97 (m, 1H), 3.10-3.20 (m, 2H), 3.3-3.47 (m, 3H), 7.36 (d, 4H), 7.70 (s, 4H), 7.79 (d, 2H), 8.02 (d, 2H), 10.35 (s, 1H).

### Example 26

### Preparation of 4-[4′-({[(2-chlorophenyl)amino]carbon}amino-1,1′-biphenyl-4-yl]-2-(2-methoxyethyl)-4-oxobutanoic acid

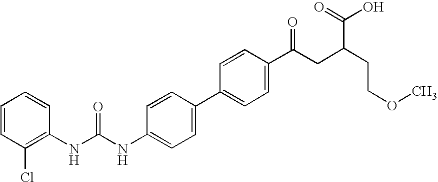

To a standard amber 4 mL vial was added methyl 2-[2-(4′-amino-1,1′-biphenylyl)-2-oxoethyl]pentanoate (30 mg, 0.08 mmol, prepared as described in US 2004/0224997), 2-chlorophenyl isocyanate (19 mg, 0.13 mmol), and dichloromethane (2 mL). The resulting solution was stirred for 16 h and was then filtered. The filtrate was concentrated under reduced pressure and the residue was dissolved in methanol (1 mL) and tetrahydrofuran (1 mL). An aqueous sodium hydroxide solution (1 N, 0.28 mL) was added. The reaction mixture was stirred at rt for 16 h and was then concentrated under reduced pressure. The residue was purified by preparative reverse-phase HPLC (water/acetonitrile gradient, containing 0.11% trifluoroacetic acid) to 4-[4′-({[(2-chlorophenyl)amino]carbonyl}amino)-1,1′-biphenyl-4-yl]-2-(2-methoxyethyl)-4-oxobutanoic acid (15 mg, 32%). LC-MS RT=3.19 min., m/z 481.0 (MH⁺) ; ¹H NMR (300 MHz, DMSO-d₆) δ 1.67-1.95 (2 m, 2H), 2.85-2.97 (m, 1H), 3.10-3.20 (m, 2H), 3.23 (s, 3H), 3.35-3.49 (m, 2H), 7.03 (t, 1H), 7.3 (t, 1H), 7.45 (d, 1H), 7.59 (d, 2H), 7.73 (d, 2H), 7.80 (d, 2H), 8.02 (d, 2H), 8.16 (d, 1H), 8.35 (s, 1H), 9.59 (s, 1H), 12.13 (br s, 1H).

### Example 27

### Preparation of 4-(4′-{[(3,5-difluorophenyl)acetyl]-amino}-1,1′-biphenyl-4-yl)-2,2-dimethyl-4-oxobutanoic acid

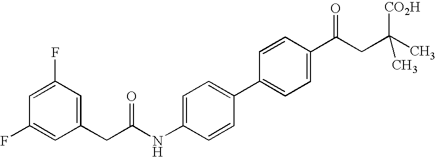

To a solution of ethyl 4-(4′-amino-1,1′-biphenyl-4-yl)-2,2-dimethyl-4-oxobutanoate (60.0 mg, 0.190 mmol, prepared as described in US 2004/0224997) in dichloromethane (4.0 mL) was added 3,5-difluorophenylacetyl chloride (55.1 mg, 0.290 mmol) and PS-DIEA (80 mg, 0.38 mmol). The solution/suspension was stirred at rt overnight. The PS-DIEA polymer was removed by filtration and the filtrate was concentrated under reduced pressure. The residue was dissolved in 1:1 methanol/tetrahydrofuran (1.2 mL), aqueous sodium hydroxide (1 N, 0.3 mL) was added, and the reaction mixture was stirred overnight at rt. The mixture was filtered through a 0.45μ PTFE filter and purified by reverse-phase HPLC using 20%-80% gradient acetonitrile/water containing 0.1% trifluoroacetic acid. The combined HPLC fractions containing the required acid were concentrated under reduced pressure to give 4-(4′-{[(3,5-difluorophenyl)acetyl]amino}-1,1′-biphenylyl)-2,2-dimethyl-4-oxobutanoic acid as a white solid (48.9 mg, 84%). LC-MS: RT=3.25 min; m/z 452.2 (MH⁺); ¹H NMR (400 MHz, DMSO-d₆) δ 1.20 (s, 6H), 3.32 (s, 2H), 3.77 (s, 2H), 7.01-7.18 (m, 3H), 7.72 (s, 4H), 7.78 (d, 2H), 7.99 (d, 2H), 10.7 (s, 1H), 11.98 (br s, 1H).

### Example 28

### 4-[4′-({[(3,4-Dimethylphenyl)amino]carbonyl}amino)-1,1′-biphenyl-4-yl]-2,2-dimethyl-4-oxobutanoic acid

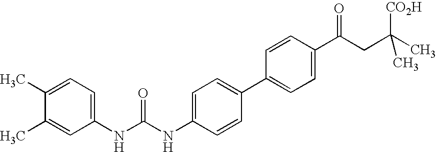

To a solution of ethyl 4-(4′-amino-1,1′-biphenyl-4-yl)-2,2-dimethyl-4-oxobutanoate (20.0 mg, 0.0600 mmol, prepared as described in US 2004/0224997) in dichloromethane (1.0 mL) was added 3,4-dimethylphenylisocyanate (14 mg, 0.090 mmol), and the solution was stirred at rt overnight. The mixture was concentrated under reduced pressure and the residue was dissolved in 1:1 methanol/tetrahydrofuran (0.8 mL). Aqueous sodium hydroxide (1 N, 0.3 mL) was added and the reaction mixture was stirred overnight at rt. The reaction mixture was filtered through a 0.45μ PTEE filter and purified by reverse-phase HPLC using 20%-80% gradient acetonitrile/water containing 0.1% trifluoroacetic acid. The combined HPLC fractions containing the required acid were concentrated under reduced pressure to give 4-[4′-([(3,4-dimethylphenyl)amino]carbonyl}amino)-1,1′-biphenyl-4-yl]-2,2-dimethyl oxobutanoic acid as a white solid (3.5 mg, 13%). LC-MS: RT=3.39 min; m/z 445.3 (MH⁺); ¹H NMR (400 MHz, DMSO-d₆) δ 1.23 (s, 6H), 2.17 (s, 3H), 2.19 (s, 3H), 3.34 (s, 2H (overlaps with H₂O signal), 7.01 (d, 1H), 7.17 (d, 1H), 7.25 (s, 1H), 7.58 (d, 2H), 7.67 (d, 2H), 7.78 (d, 2H), 7.99 (d, 2H), 8.62 (br s, 1H), 8.89 (br s, 1H).

### Example 29

### Preparation of 4-(4′-{[(5-methoxy-1H-indol-2-yl)carbonyl]amino}-1,1′-biphenyl-4-yl)-2,2-dimethyl-4-oxobutanoic acid

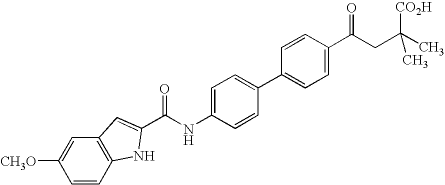

To a solution of 5-methoxyindole-2-carboxylic acid (61.4 mg, 0.32 mmol) in N,N-dimethylformamide (1.0 mL) were added 1-hydroxybenzotriazole hydrate (86.8 mg, 0.640 mmol) and N′-(3-dimethylaminopropyl)-N-ethylcarbodimide hydrochloride (86.2 mg, 0.450 mmol), followed by a solution of ethyl 4-(4′-amino-1,1′-biphenyl-4-yl)-2,2-dimethyl-4-oxobutanoate (100 mg, 0.320 mmol, prepared as described in US 2004/0224997) in N,N-dimethylformamide (1.0 mL). The solution was stirred at rt overnight. Water (4.0 mL) was added and the mixture was extracted three times with ethyl acetate (3 mL each extraction). The combined extracts were concentrated under reduced pressure and the residue was dissolved in 1:1 methanol/tetrahydrofuran (1.0 mL). Aqueous sodium hydroxide (1 N, 0.5 mL) was added and the reaction mixture was stirred overnight at rt The reaction mixture was filtered through a 0.45μ PTFE filter and purified by reverse-phase HPLC using 20%-80% gradient acetonitrile/water containing 0.1% trifluoroacetic acid. The combined HPLC fractions containing the required acid were concentrated under reduced pressure to give 4-(4′-{[(5-methoxy-1H-indol-2-yl)carbonyl]amino}-1,1′-biphenyl-4-yl)-2,2-dimethyl-4-oxobutanoic acid as a white solid (44.0 mg, 29%). LC-MS: RT=3.19 min; m/z 471.0 (MH⁺); ¹H NMR (400 MHz, DMSO-d₆) δ 1.25 (s, 6H), 3.33 (s, 2H), 3.80 (s, 3H), 6.89 (d, 1H), 7.15 (s, 1H), 7.32-7.42 (m, 2H), 7.79 (d, 2H), 7.83 (d, 2H), 7.94 (d, 2H), 8.04 (d, 2H), 10.32 (s, 1H), 11.62 (s, 1H), 11.97 (br s, 1H).

### Example 30

### Preparation of 4-{4′-[(1,3-dihydro-2H-isoindol-2-ylcarbonyl)amino]-1,1′-biphenyl-4-yl}-2,2-dimethyl-4-oxobutanoic acid

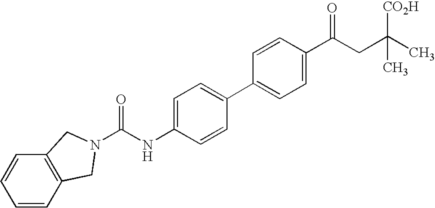

### Step 1. Preparation of methyl 4-{4′-[(1,3-dihydro-2H-isoindol-2-ylcarbonyl)amino]-1,1′-biphenyl-4-yl}-2,2-dimethyl-4-oxobutanoate

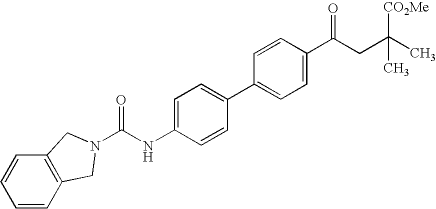

In an argon filled three-necked round bottom flask, a suspension of methyl 4-(4′-amino-1,1′-biphenyl-4-yl)-2,2-dimethyl-4-oxobutanoate (0.23 g, 0.74 mmol, prepared as described in US 2004/0224997) in toluene (3.2 mL) was treated with triethylamine (1.0 mL) and cooled to 0° C. The three-necked flask was vented to a 2 N aqueous solution of sodium hydroxide. The stirred suspension was slowly treated with phosgene (20% in toluene, 13.0 mL, 81.0 mmol) and then was stirred at rt for 2 h. The suspension was filtered to remove salts and concentrated under reduced pressure to give methyl 4-(4′-isocyanato-1,1′-biphenylyl)-2,2-dimethyl-4-oxobutanoate as a dark, orange oil. The oil was dissolved in 1,2-dichloroethane (12.0 mL) and used immediately in subsequent reactions. A fraction of this isocyanate solution (2 mL, ca. 0.12 mmol) was treated with isoindoline (0.02 g, 0.18 mmol) and then was stirred at rt for 16 h. The mixture was concentrated under reduced pressure, and the crude solid was triturated with ethyl acetate. The mixture was filtered to give the title compound as a white solid (0.04 g, 73%). ¹H NMR (300 MHz, DMSO-d₆) δ 1.23 (s, 6H), 3.40 (s, 2H), 3.55 (s, 3H), 4.79 (s, 4H), 7.35-7.32 (m, 4H), 7.72-7.70 (m, 4H), 7.81 (d, 2H), 8.00 (d, 2H), 8.53 (s, 1H); LC-MS ret. time=3.38 min, m/z 457.1 (MH⁺).

### Step 2. Preparation of 4-{4′-[(1,3-dihydro-2H-isoindol-2-ylcarbonyl)amino]-1,1′-biphenyl-4-yl}-2,2-dimethyl-4-oxobutanoic acid

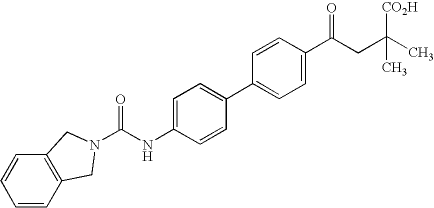

To a solution of 4-[4′-[(1,3-dihydro-2H-isoindol-2-ylcarbonyl)amino]-1,1′-biphenyl-4-yl]-2,2-dimethyl-4-oxobutanoate in methanol (2.0 mL) and tetrahydrofuran (1.0 mL) was added 2 N sodium hydroxide solution (2.0 mL) and stirred at rt for 16 h. The reaction was then diluted with water and the pH of the aqueous mixture was adjusted to 2. The product was extracted with ethyl acetate. The organic layer was then washed with saturated sodium chloride solution, dried over anhydrous magnesium sulfate and concentrated under reduced pressure to give a white solid (0.040 g, 97%). LC-MS ret. time=3.01 min, m/z 443.2 (MH⁺; ¹H NMR (300 MHz, DMSO-d₆) δ 1.22 (s, 6H), 4.79 (s, 4H), 7.38-7.29 (m, 4H), 7.75-7.67 (m, 4H), 7.80 (d, 2H), 8.00 (d, 2H), 8.53 (s, 1H), 11.95 (s, 1H).

### Example 31

### Preparation of 4-(2-{4′-[(4-fluoro-3-methylbenzoyl)amino]-1,1′-biphenyl-4-yl}-2-oxoethyl)tetrahydro-2H-pyran-4-carboxylic acid

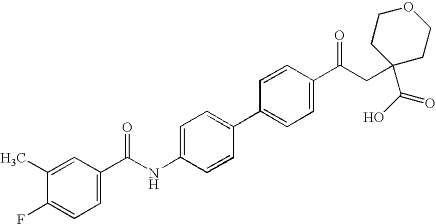

To a solution of methyl 4-[2-(4′-amino-1,1′-biphenylyl)-2-oxoethyl]tetrahydro-2H-pyran-4-carboxylate (40 mg, 0.11 mmol, prepared as described in US 2004/0224997) and 4-fluoro-3-methylbenzoyl chloride (24 mg, 0.14 mmol) in dichloromethane (2 mL) was added poly-4-vinyl pyridine (38 mg, 0.34 mmol). The resulting suspension was stirred at rt for 16 h. The mixture was then filtered and the filtrate was concentrated under redued pressure. The residue was dissolved in methanol (1 mL) and tetrahydrofuran (1 mL) and a 1.0 N aqueous solution of sodium hydroxide (0.5 mL, 0.5 mmol) was added. The mixture was stirred at rt for 16 h, and then concentrated under reduced pressure. The residue was purified by preparative reverse-phase HPLC (water/acetonitrile gradient, containing 0.1% TFA) to afford 4-(2-{4′-[(4-fluoro-3-methylbenzoyl)amino]-1,1′-biphenyl-4-yl}-2-oxoethyl)tetrahydro-2H-pyran-4-carboxylic acid (11.9 mg, 22%). LC-MS m/z 476.0, RT=3.1 min; ¹H NMR (300 MHz, DMSO-d₆) δ 1.62-1.66 (m, 2H), 1.95-1.99 (m, 2H), 2.33 (s, 3H), 3.46 (s, 2H), 3.59-3.67 (m, 4H), 7.30 (t, 1H), 7.78 (d, 2H), 7.81-7.91 (m, 4H), 7.93 (d, 2H), 8.02 (d, 2H), 10.35 (s, 1H).

### Example 32

### Preparation of 4-{2-[4′-({[(2-ethoxyphenyl)amino]carbonyl}amino)-1,1′-biphenyl-4-yl]-2-oxoethyl}tetrahydro-2H-pyran-4-carboxylic acid

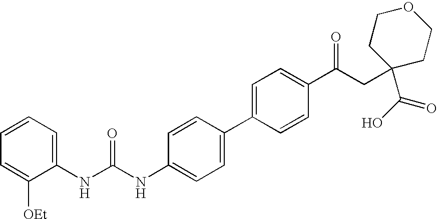

A mixture of methyl 4-[2-(4′-amino-1,1′-biphenyl-4-yl)-2-oxoethyl]tetrahydro-2H-pyran-4-carboxylate (40 mg, 0.11 mmol, prepared as described in US 2004/0224997) and 2-ethoxyphenyl isocyanate (22 mg, 0.14 mmol) in dichloromethane (2 mL) was stirred at rt for 16 h. The mixture was concentrated under reduced pressure and the residue was dissolved in tetrahydrofuran (1 mL) and methanol (1 mL). Aqueous sodium hydroxide (1 N, 0.5 mL, 0.5 mmol) was then added. The mixture was then stirred at rt for 16 h and then concentrated under reduced pressure. The residue was purified by preparative reverse-phase HPLC (water/acetonitrile gradient, containing 0.1% TFA) to afford 4-{2-[4′-({[(2-ethoxyphenyl)amino]carbonyl}amino)-1,1′-biphenyl-4-yl]-2-oxoethyl}tetrahydro-2H-pyran-4-carboxylic acid (9.1 mg, 16%). LC-MS m/z 503.2 (MH⁺), RT=3.11 min; ¹H NMR (300 MHz, DMSO-d₆) δ 11.43 (t, 3H), 1.61-1.67 (m, 2H), 1.94-1.99 (m, 2H), 3.45 (s, 2H), 3.57-3.69 (m, 4H), 4.13 (q, 2H), 6.86-6.94 (m, 2H), 7.01 (d, 2H), 7.60 (d, 2H), 7.71 (d, 2H), 7.79 (d, 2H), 8.01 (d, 2H), 8.13 (d, 2H), 9.57 (s, 1H).

### Example 33

### Preparation of 1-{2-[4′-({[(2-chlorophenyl)amino]carbonyl}amino)biphenyl-4-yl]-2-oxoethyl}cyclopentanecarboxylic acid

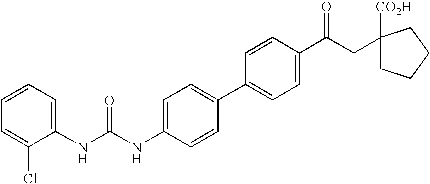

To a solution of methyl 1-[2-(4′-aminobiphenyl-4-yl)-2-oxoethyl]cyclopentanecarboxylate (38.4 mg, 0.11 mmol, prepared as described in US 2004/0224997) in dichloroethane (2 mL) was added 2-chlorophenyl isocyanate (21.0 mg, 0.14 mmol), and the resulting solution was stirred at rt for 16 h. The mixture was evaporated to dryness, and the residue was dissolved in MeOH (1.0 mL) and THF (1.0 mL). Aqueous NaOH (1 N, 0.33 mL, 0.33 mmol) was added, and the resulting mixture was stirred at rt for 16 h. The reaction mixture was filtered and then purified by preparative reverse-phase HPLC (water/acetonitrile gradient, containing 0.1% TFA) to afford 1-{2-[4′-({[(2-chlorophenyl)amino]carbonyl}amino)biphenyl-4-yl]-2-oxoethyl}cyclopentanecarboxylic acid (20 mg, 38%). LC-MS m/z 477.2 (MH⁺), RT=3.52 min; ¹H NMR (300 MHz, DMSO-d₆) δ 1.50-1.69 (m, 6H), 2.03-2.16 (m, 2H), 3.43 (s, 2H), 6.96-7.05 (m, 1H), 7.24-7.36 (m, 1H), 7.48 (d, 1H), 7.57 (d, 2H), 7.72 (d, 2H), 7.78 (d, 2H), 7.98 (d, 2H), 8.16 (d, 1H), 8.35 (s, 1H), 9.56 (s, 1H), 11.85 (s, 1H).

### Example 34

### Preparation of trans-2-({4′-[(4-chlorobenzoyl)amino]-1,1′-biphenyl-4-yl}carbonyl)-cyclohexanecarboxylic acid

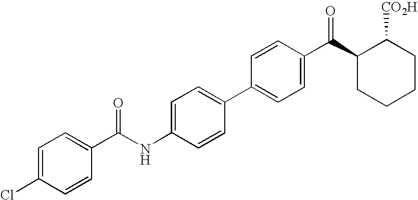

To a solution of methyl cis-2-[(4′-amino-1,1′-biphenyl-4-yl)carbonyl]cyclohexane-carboxylate (50 mg, 0.15 mmol, prepared as described in US 2004/0224997) in dichloromethane (2 mL) was added 4-chlorobenzoyl chloride (51.87 mg, 0.30 mmol) and triethylamine (75.27 mg, 0.74 mmol), and the resulting solution was stirred at rt for 72 h. The mixture was evaporated to dryness. The residue was dissolved in MeOH and NaOH (1 N, 1.5 mL, 1.5 mmol) was then added and the solution was stirred at 60° C. overnight. Solvent was removed under reduced pressure, HCl (2 N) was added, and then MeOH was added to dissolve the precipitate. The solution was purified by preparative reverse-phase HPLC (water/acetonitrile gradient, containing 0.1% TFA) to afford trans-2-({4′-[(4-chlorobenzoyl)amino]-1,1′-biphenyl-4-yl}carbonyl)cyclohexanecarboxylic acid (3.4 mg, 5%). LC-MS RT=3.48 min, m/z 462.1 (MH⁺); ¹H NMR (400 MHz, MeOH-d₄) δ 1.27 (m, 1H), 1.35˜1.57 (m, 3H), 1.88 (m, 2H), 2.06 (m, 1H), 2.23 (m, 1H), 2.84 (m, 1H), 3.68 (m, 1H), 7.54 (m, 2H), 7.73 (m, 2H), 7.78 (d, 2H), 7.84 (d, 2H), 7.94 (m, 2H), 8.07 (d, 2H).

### Example 35

### Preparation of trans-2-{[4′-({[(2,4-difluorophenyl)amino]carbonyl}amino)-1,1′-biphenyl-4-yl]carbonyl}cyclohexanecarboxylic acid

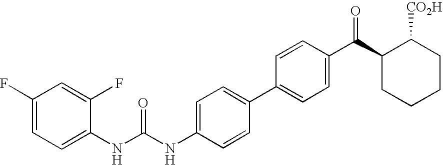

To a solution of methyl cis-2-[(4′-amino-1,1′-biphenylyl)carbonyl]cyclohexane-carboxylate (50 mg, 0.15 mmol, prepared as described in US 2004/0224997) in dichloromethane (2 mL) was added 2,4-difluorophenyl isocyanate (46 mg, 0.30 mmol), and the resulting solution was stirred at rt overnight. The mixture was evaporated to dryness and the residue was suspended in ether. The precipitate was collected by filtration and washed with ether and dried under high vacuum to give methyl 2-{[4′-({[(2,4-difluorophenyl)amino]carbonyl}amino)-1,1′-biphenyl-4-yl]carbonyl}-cyclohexanecarboxylate (28 mg, 36%). LC-MS RT=3.84 min, m/z 493.0 (MH⁺). A sample of this intermediate (24 mg, 0.05 mmol) was mixed with MeOH and the suspension was heated at 50° C. to effect dissolution. Then aqueous NaOH (1 N, 0.5 mL, 0.5 mmol) was added to the solution and the mixture was stirred at 50° C. overnight. The mixture was then concentrated under reduced pressure and the residue was dissolved in water. HCl (conc.) was gradually added with stirring until the mixture was acidic. The solution was purified by preparative reverse-phase HPLC (water/acetonitrile gradient, containing 0.1% TFA) to afford trans-2-([4′-({[(2,4-difluorophenyl)amino]carbonyl}amino)-1,1′-biphenyl-4-yl]carbonyl}cyclohexanecarboxylic acid (6.5 mg, 28%). LC-MS RT=3.34 min, m/z 479.2 (MH⁺); ¹H NMR (400 MHz, MeOH-d₄) δ 1.26 (m, 1H), 1.37-1.59 (m, 3H), 1.89 (m, 2H), 2.06 (m, 1H), 2.23 (m, 1H), 2.84 (m, 1H), 3.67 (m, 1H), 6.94 (m, 1H), 7.03 (m, 1H), 7.56 (m, 2H), 7.66 (m, 2H), 7.75 (m, 2H), 7.99˜8.07 (m, 3H).

### Example 36

### Preparation of trans-2-{[-4′-(Pentanoylamino)-1,1′-biphenyl-4-yl]carbonyl}cyclopropane-carboxylic acid

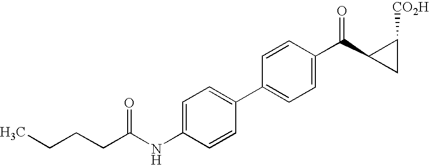

### Step 1. Preparation of methyl trans-2-{[4′-(pentanoylamino)-1,1′-biphenyly-4-yl]carbonyl}-cyclopropanecarboxylate

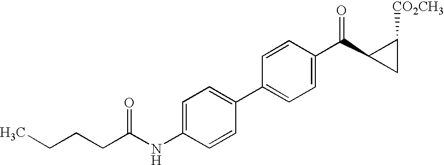

To a solution of methyl trans-2-[(4′-amino-1,1′-biphenylyl)carbonyl]-cyclopropane-carboxylate (45 mg, 0.15 mmol, prepared as described in US 2004/0224997) in dichloromethane (2 mL) was added butyryl chloride (36.7 mg, 0.30 mmol) and triethylamine (46.7 mg, 0.46 mmol), and the resulting solution was stirred at rt overnight. The mixture was evaporated to dryness under reduced pressure and the residue was suspended in ether. The precipitate was collected by filtration and washed with ether and dried under high vacuum to give methyl trans-2-{[4′-(pentanoylamino) 1,1′-biphenyl-4-yl]carbonyl}cyclopropane-carboxylate (26.4 mg, 45%). LC-MS RT=3.25 min, m/z 380.3 (MH⁺); ¹H NMR (400 MHz, DMSO-d₆) δ 0.90 (t, 3H), 1.33 (sex, 2H), 1.50˜1.62 (m, 3H), 2.22 (m, 1H), 2.33 (t, 2H), 3.66 (s, 3H), 7.71 (s, 4H), 7.81 (d, 2H), 8.09 (d, 2H), 10.0 (s, 1H).

### Step 2. Preparation of trans-2-{[4′-(pentanoylamino)-1,1′-biphenyl-4-yl]carbonyl}cyclo-propanecarboxylic acid

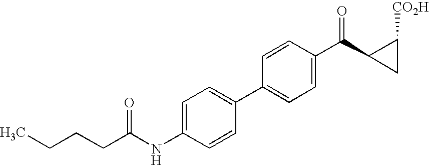

Methyl trans-2-{[4′-(pentanoylamino)-1,1′-biphenyl-4-yl]carbonyl}cyclopropanecarboxylate (24.1 mg, 0.06 mmol) was mixed with MeOH and the suspension was heated at 50° C. to effect dissolution. Then aqueous NaOH (1 N, 1 mL, 1 mmol) was added to the solution and the mixture was stirred at 50° C. overnight. The reaction mixture was concentrated under reduced pressure and the residue was suspended in water. HCl (conc.) was gradually added with stirring until the mixture was acidic, and the precipitate that formed was collected by filtration, washed with ether and dried under high vacuum to give trans-2-{[4′-(pentanoylamino)-1,1′-biphenyl-4-yl]carbonyl}cyclopropanecarboxylic acid (13.4 mg, 57%). LC-MS RT=2.91 min, m/z 366.2 (MH⁺); ¹H NMR (400 MHz, DMSO-d₆) δ 0.69 (t, 3H), 1.11 (m, 2M), 1.27 (m, 2H), 1.37 (m, 2M), 1.89 (m, 1H), 2.11 (t, 2H), 3.03 (m, 1H), 7.49 (s, 4H), 7.60 (d, 2H), 7.87 (d, 2H), 9.80 (s, 1H), 12.36 (s, 1H).

### Example 37

### Preparation of trans-2-[(4′-{[(3,4-difluorophenyl)acetyl]amino}-1,1′-biphenyl-4-yl)carbonyl]cyclopropanecarboxylic acid

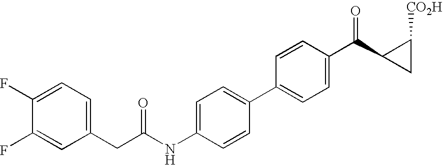

To a solution of methyl trans-2-[(4′-amino-1,1′-biphenyl-4-yl)carbonyl]cyclopropane-carboxylate (94 mg, 0.32 mmol, prepared as described in US 2004/0224997) in dichloromethane (3 mL) was added 3,4-difluorophenylacetic acid (65.7 mg, 0.38 mmol), dimethylaminopyridine (1.9 mg, 0.02 mmol), EDCI (73.2 mg, 0.38 mmol), and the resulting solution was stirred at rt for 3 days. Water was added and the mixture was extracted with DCM. The combined organic layers were washed with aqueous NaOH (1 N), HCl (1 N), water, and brine, dried over Na₂SO₄, filtered and concentrated under reduced pressure. The residue was mixed in aqueous HCl (1 N) and filtered. The precipitate was washed with water, ether and dried in a vacuum oven to give methyl trans-2-[(4′-{[(3,4-difluorophenyl)acetyl]amino}-1,1′-biphenyl-4-yl)carbonyl]cyclopropane-carboxylate (63.6 mg, 44%). LC-MS RT=3.64 min, m/z 450 (MHz; ¹H NMR (400 MHz, DMSO-d₆) δ 1.62 (m, 2H), 2.36 (m, 1H), 3.32 (m, 1H), 3.74 (s, 5H), 7.12 (m, 1H), 7.18-7.30 (m, 2H), 7.62 (m, 4H), 7.72 (m, 2H), 8.08 (m, 2H). A sample of this intermediate (63 mg, 0.14 mmol) was mixed with MeOH and the suspension was heated at 50° C. to effect dissolution. Then aqueous NaOH (1 N, 1.5 mL, 1.5 mmol) was added to the solution and the mixture was stirred at 50° C. overnight. The reaction mixture was concentrated under reduced pressure and the residue was suspended in water. HCl (conc.) was gradually added with stirring until the mixture was acidic and the precipitate that formed was collected by filtration, washed with ether and dried under high vacuum to give trans-2-[(4′-{[(3,4-difluorophenyl)acetyl]amino}-1,1′-biphenyl-4-yl)carbonyl]cyclopropanecarboxylic acid (15.8 mg, 25%). LC-MS RT=3.01 min, m/z 436.1 (MH⁺); ¹H NMR (400 MHz, DMSO-d₆) δ 1.47 (m, 2H), 2.09 (m, 1H), 3.24 (m, 1H), 3.69 (s, 2H), 7.16 (m, 1H), 7.38 (m, 2H), 7.72 (m, 4H), 7.82 (m, 2H), 8.09 (m, 2H), 10.34 (s, 1H).

### Example 38

### Preparation of trans-2-{[4′-({[(2-chlorophenyl)amino]carbonyl}amino)-1,1′-biphenyl-4-yl]carbonyl}cyclopropanecarboxylate

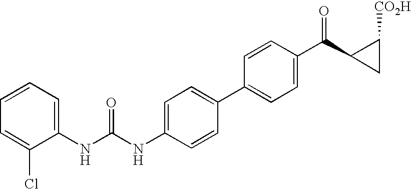

To a solution of methyl trans-2-[(4′-amino-1,1′-biphenyl-4-yl)carbonyl]cyclopropane-carboxylate (45 mg, 0.15 mmol, prepared as described in US 2004/0224997) in dichloromethane (2 mL) was added 2-chlorophenyl isocyanate (46.8 mg, 0.30 mmol), and the resulting solution was stirred at rt overnight. The mixture was evaporated to dryness and the residue was suspended in ether. The precipitate was collected by filtration and washed with ether and dried under high vacuum to give methyl trans-2-{[4′-({[(2-chlorophenyl)amino]carbonyl}amino)-1,1′-biphenyl-4-yl]carbonyl}-cyclopropanecarboxylate (22.7 mg, 33%). LC-MS RT=3.91 min, m/z 450 (MH⁺). A sample of this intermediate (24.3 mg, 0.05 mmol) was mixed with MeOH and the suspension was heated at 50° C. to effect dissolution. Then aqueous NaOH (1N, 0.5 mL, 0.5 mmol) was added to the solution and the mixture was stirred at 50° C. overnight. The reaction mixture was concentrated under reduced pressure and the residue was dissolved in water. HCl (conc.) was gradually added with stirring until the mixture was acidic. The solution was extracted with EtOAc and the combined organic layers were washed with water, brine, dried over Na₂SO₄ and concentrated under reduced pressure to give trans-2-{[4′-({[(2-chlorophenyl)amino]carbonyl}amino)-1,1′-biphenyl-4-yl]carbonyl}cyclopropanecarboxylic acid (23.5 mg, 99%). LC-MS RT=3.32 min, m/z 435.0 (MH⁺); ¹H NMR (400 MHz, DMSO-d₆) δ 1.48 (m, 2H), 2.10 (m, 1H), 3.25 (m, 1H), 7.02 (m, 1H), 7.29 (m, 1H), 7.45 (m, 1H), 7.59 (d, 2H), 7.73 (d, 2H), 7.82 (d, 2H), 8.09 (d, 2H), 8.15 (m, 1H), 8.35 (s, 1H), 9.59 (s, 1H).

### Example 39

### Preparation of trans-2-[(4′-{[(3-pyridinylamino)carbonyl]amino}-1,1′-biphenyl-4-yl)carbonyl]cyclopropanecarboxylic acid (trifluoroacetate salt)

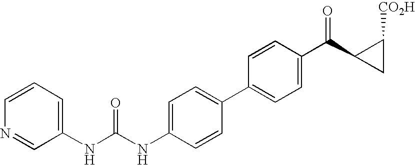

To a solution of methyl trans-2-[(4′-amino-1,1′-biphenylyl)carbonyl]cyclopropane-carboxylate (45 mg, 0.15 mmol, prepared as described in US 2004/0224997) in dichloromethane (2 mL) was added 3-pyridyl isocyanate (92 mg, 0.76 mmol), and the resulting solution was stirred at rt overnight. The mixture was evaporated to dryness under reduced pressure and the residue was dissolved in MeOH and aqueous NaOH (1 N, 0.5 mL, 0.5 mmol) was added to the solution and the mixture was stirred at 50° C. overnight. The reaction mixture was concentrated under reduced pressure and the residue was dissolved in water. HCl (conc.) was gradually added with stirring until the mixture was acidic. The solution was extracted with EtOAc and the combined organic layers were washed with water, brine, dried over Na₂SO₄ and concentrated down. The residue was dissolved in MeOH and purified by preparative reverse-phase HPLC (water/acetonitrile gradient, containing 0.1% TFA) to afford trans-2-[(4-{[(3-pyridinylamino)carbonyl]amino}-1,1′-biphenyl]yl)carbonyl]-cyclopropanecarboxylic acid (trifluoroacetate salt) (15.4 mg, 26%). LC-MS RT=2.14 min, m/z 402.1 (MH⁺); ¹H NMR (400 MHz, DMSO-d₆) δ 1.60 (m, 2H), 2.26 (m, 1H), 3.29 (m, 1H), 7.63 (m, 2H), 7.70 (m, 2H), 7.80 (m, 2H), 7.91 (m, 1H), 8.11 (m, 2M), 8.36 (m, 1H), 8.42 (m, 1H), 9.26 (m, 1H).

### Example 40

### Preparation of trans-2-[(4′-{[(4-isopropylphenyl)acetyl]amino}-1,1′-biphenyl-4-yl)carbonyl]cyclobutanecarboxylic acid

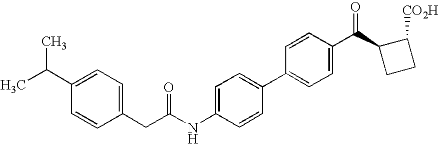

To a solution of methyl trans-2-[(4′-amino-1,1′-biphenyl-4-yl)carbonyl]cyclobutane-carboxylate (100 mg, 0.32 mmol, prepared as described in US 2004/0224997) in dichloromethane (3 mL) was added 4-isopropylphenylacetic acid (89.1 mg, 0.39 mmol), dimethylaminopyridine (1.97 mg, 0.02 mmol), EDCI (92.95 mg, 0.48 mmol), and the resulting solution was stirred at rt for 3 days. Water was added and the mixture was extracted with DCM. The combined organic layers were washed with aqueous NaOH (1 N), HCl (1 N), water, and brine, dried over Na₂SO₄, filtered and concentrated under reduced pressure to give methyl trans-2-[(4′-{[(4-isopropylphenyl)acetyl]amino}-1,1′-biphenyl-4-yl)carbonyl]cyclobutane-carboxylate as an oil. LC-MS RT=3.80 min, m/z 470.1 (MH⁺); ¹H NMR (400 MHz, CD₃OD) δ 1.24 (d, 6H), 2.20 (m, 2H), 2.35 (m, 2H), 2.90 (m, 1H), 3.61 (m, 1H), 3.65 (s, 2H), 3.70 (s, 3H), 4.38 (q, 1H), 7.19 (d, 2H), 7.26 (d, 2H), 7.67 (m, 4H), 7.77 (d, 2H), 8.01 (d, 2H). A sample of this intermediate (90 mg, 0.19 mmol) was mixed with MeOH and the suspension was heated at 50° C. to effect dissolution. Then aqueous NaOH (1 N, 2.0 mL, 2.0 mmol) was added to the solution and the mixture was stirred at 50° C. overnight. The reaction mixture was concentrated under reduced pressure and the residue was suspended in water. HCl (conc.) was gradually added with stirring until the mixture was acidic and the precipitate that formed was collected by filtration, washed with ether and purified by preparative HPLC to give trans-2-[(4′-{[(4-isopropylphenyl)acetyl]amino}-1,1′-biphenyl-4-yl)carbonyl]cyclobutanecarboxylic acid (38.7 mg, 44%). LC-MS RT=3.44 min, m/z 456.1 (M); ¹H NMR (400 MHz, DMSO-d₆) δ 1.19 (d, 6H), 2.11 (m, 3H), 2.30 (m, 1H), 2.83 (m, 1H), 3.40 (m, 1H), 3.60 (s, 2H), 4.27 (q, 1H), 7.16 (d, 2H), 7.22 (d, 2H), 7.71 (m, 4H), 7.79 (d, 2H), 7.96 (d, 2H), 10.25 (s, 1H), 12.23 (bs, 1H).

### Example 41

### Preparation of trans-2-{[-4′-({[(2-ethoxyphenyl)amino]carbonyl}amino)-1,1′-biphenyl-4-yl]carbonyl}cyclopentanecarboxylate

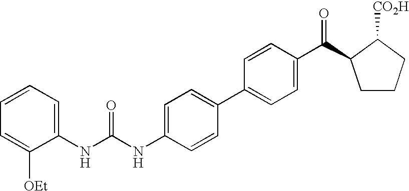

To a solution of methyl trans-2-[(4′-amino-1,1′-biphenylyl)carbonyl]cyclopentane-carboxylate (47 mg, 0.15 mmol, prepared as described in US 2004/0224997) in dichloromethane (2 mL) was added 2-ethoxyphenyl isocyanate (47.43 mg, 0.30 mmol), and the resulting solution was stirred at rt overnight. The mixture was evaporated to dryness under reduced pressure and the residue was suspended in ether. The precipitate was collected by filtration, washed with ether and dried under high vacuum to give methyl trans-2-{[4′-({[(2-ethoxyphenyl)amino]carbonyl}amino)-1,1′-biphenyl-4-yl]carbonyl}cyclopentanecarboxylate (24.7 mg, 34%). LC-MS RT=3.80 in, m/z 487.0 (MH⁺); ¹H NMR (400 MHz, CD₂Cl₂) δ 1.37 (t, 3H), 1.67-1.90 (m, 4H), 2.11 (m, 2H), 3.36 (m, 1H), 3.57 (s, 3H), 4.04 (m, 4H), 6.69 (s, 1H), 6.82˜6.96 (m, 3H), 7.14 (s, 1H), 7.47 (d, 2H), 7.57 (d, 2H), 7.63 (d, 2H), 7.97 (d, 2H), 8.03 (d, 1H). A sample of this intermediate (24.6 mg, 0.05 mmol) was mixed with MeOH and the suspension was heated at 50° C. to effect dissolution. Then aqueous NaOH (1 N, 1.0 mL, 1.0 mmol) was added to the solution and the mixture was stirred at 50° C. overnight. The reaction mixture was concentrated under reduced pressure and the residue was suspended in water. HCl (conc.) was gradually added with stirring until the mixture was acidic, and the precipitate that formed was collected by filtration, washed with dichloromethane and dried under high vacuum to give trans-2-{[4′-({[(2-ethoxyphenyl)amino]carbonyl}amino)-1,1′-biphenyl-4-yl]carbonyl}cyclopentanecarboxylic acid (11.6 mg, 48%). LC-MS RT=3.42 min, m/z 473.2 (MH⁺); ¹H NMR (400 MHz, DMSO-d₆) δ 1.41 (t, 3H), 1.53˜1.84 (m, 4H), 1.98 (m, 1H), 2.15 (m, 1H), 3.21 (m, 1H), 4.13 (m, 3H), 6.90 (m, 2H), 7.00 (m, 1H), 7.60 (d, 2H), 7.71 (d, 2H), 7.80 (d, 2H), 8.05 (d, 2H), 8.13 (m, 2H), 9.58 (s, 1H), 12.18 (s, 1H).

### Example 42

### Preparation of trans-2-{[4′-({[(2,4-difluorophenyl)amino]carbonyl}amino)-1,1′-biphenyl-4-yl]carbonyl}cyclopentanecarboxylic acid

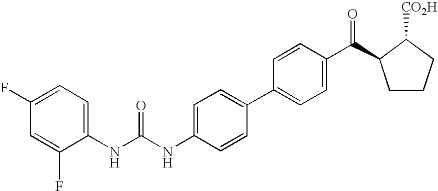

To a solution of methyl trans-2-[(4′-amino-1,1′-biphenyl-4-yl)carbonyl]cyclopentane-carboxylate (47 mg, 0.15 mmol, prepared as described in US 2004/0224997) in dichloromethane (2 mL) was added 2,4-difluoro isocyanate (45 mg, 0.30 mmol) and the resulting solution was stirred at rt overnight. The mixture was evaporated to dryness under reduced pressure, and the residue was dissolved in MeOH. Aqueous NaOH (1N, 0.5 mL, 0.5 mmol) was added to the solution and the mixture was stirred at 50° C. overnight. The reaction mixture was concentrated under reduced pressure and the residue was dissolved in water. HCl (conc.) was gradually added with stirnng until the mixture was acidic. The solution was extracted with EtOAc and the combined organic layers were washed with water and brine, dried over Na₂SO₄, and concentrated under reduced pressure. The residue was dissolved in MeOH and purified by preparative reverse-phase HPLC (water/acetonitrile gradient, containing 0.1% TFA) to afford trans-2-{[4′-({[(2,4-difluorophenyl)amino]carbonyl}amino)-1,1′-biphenyl-4-yl]carbonyl}cyclopentanecarboxylic acid (11.6 mg, 15%). LC-MS RT=3.31 min, m/z 465.1 (MH⁺); ¹H NMR (400 MHz, MeOH-d₄) δ 1.69˜2.0 (m, 4H), 2.13 (m, 1H), 2.24 (m, 1H), 2.37 (m, 1H), 4.17 (m, 1H), 6.93 (m, 1H), 7.02 (m, 1H), 7.57 (m, 2H), 7.67 (m, 2H), 7.76 (m, 2H), 7.99˜8.08 (m, 3H).

### Example 43

### Preparation of N-[4′-(3-{[(methylsulfonyl)amino]carbonyl}-5-phenyl-pentanoyl)-1,1′-biphenyl-4-yl]pentanamide

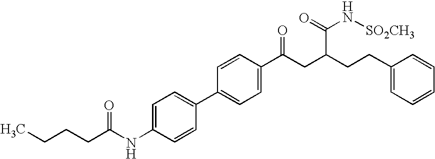

A solution of 4-oxo-4-[4′-(pentanoylamino)-1,1′-biphenyl-4-yl]-2-(2-phenylethyl)-butanoic acid (26.2 mg, 0.057 mmol, prepared as described in Example 2), methanesulfonamide (5.4 mg, 0.057 mmol), 1-ethyl-3-[3-(dimethylamino)propyl]-carbodiimide hydrochloride (11 mg, 0.057 mmol), and 4-(dimethylamino)pyridine (7 mg, 0.057 mmol) in dichloromethane (1 mL) was stirred at rt for 16 h. The reaction mixture was concentrated under reduced pressure and the crude product was purified by preparative reverse-phase HPLC (water/acetonitrile gradient, containing 0.1% TFA) to afford N-[4′-(3-{[(methylsulfonyl)amino]carbonyl}-5-phenylpentanoyl)-1,1′-biphenyl-4-yl]pentanamide (5.8 mg, 30%). LC-MS RT=3.52 min, m/z 535.1 (MH⁺); ¹H NMR (300 MHz, CDCl₃) δ 0.96 (t, 3H), 1.41 (m, 2H), 1.71 (m, 2H), 1.92 (m, 1H), 2.18 (m, 1H), 2.40 (t, 2H), 2.71-2.86 (m, 3H), 3.20 (dd, 1H), 3.28 (s, 3H), 3.53 (m, 1H), 7.19-7.34 (m, 6H), 7.56-7.66 (m, 6H), 7.95 (d, 2H), 8.52 (s, 1H).

By using the methods described above and by selecting the appropriate starting materials, other compounds of the invention were prepared and characterized. These compounds, together with Examples 1 to 43, are summarized in Tables 1 to 6.

Lengthy table referenced here
US20090215780A1-20090827-T00001
Please refer to the end of the specification for access instructions.

By using the methods described above and by selecting the appropriate starting materials, additional compounds of Formula (I) can be prepared, such as those illustrated in Table 7 below.

TABLE 7

| Entry | |
|---|---|
| No. | Structure |
| 742 | 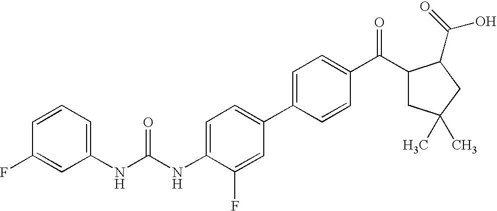 |
| 743 | 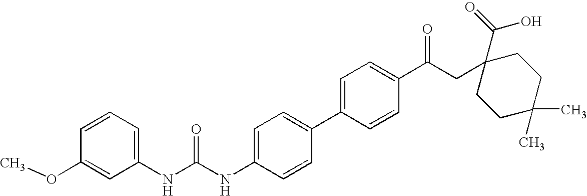 |
| 744 | 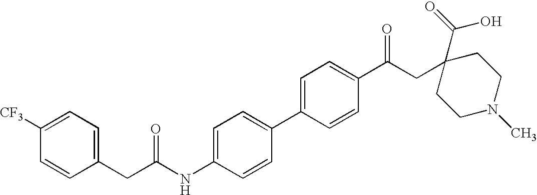 |
| 745 | 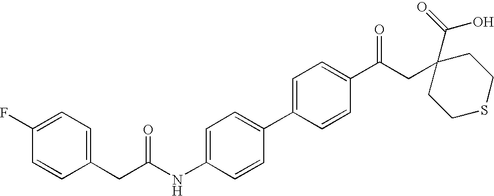 |
| 746 | 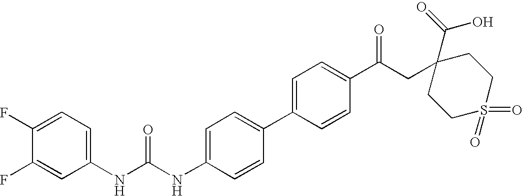 |
| 747 | 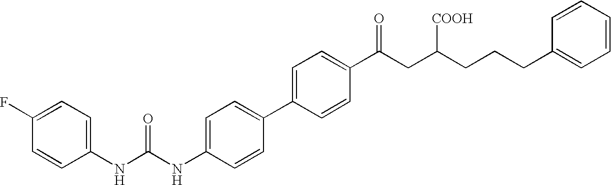 |
| 748 | 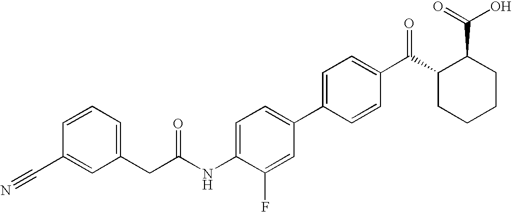 |
| 749 | 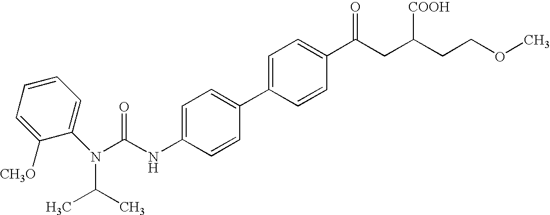 |
| 750 | 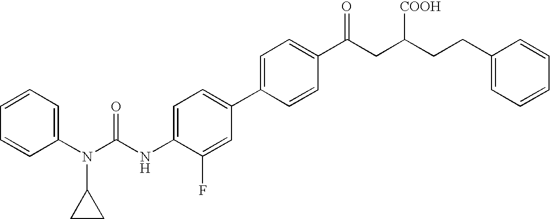 |

### Methods of Use

As used herein, various terms are defined below.

When introducing elements of the present invention or the preferred embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements.

The term “subject” as used herein includes mammals (e.g., humans and animals).

The term “treatment” includes any process, action, application, therapy, or the like, wherein a subject, including a human being, is provided medical aid with the object of improving the subject's condition, directly or indirectly, or slowing the progression of a condition or disorder in the subject.

The term “combination therapy” or “co-therapy” means the administration of two or more therapeutic agents to treat an obese condition and/or disorder. Such administration encompasses co-administration of two or more therapeutic agents in a substantially simultaneous manner, such as in a single capsule having a fixed ratio of active ingredients or in multiple, separate capsules for each inhibitor agent. In addition, such administration encompasses use of each type of therapeutic agent in a sequential manner.

The phrase “therapeutically effective” means the amount of each agent administered that will achieve the goal of improvement in an obese condition or disorder severity, while avoiding or minimizing adverse side effects associated with the given therapeutic treatment.

The term “pharmaceutically acceptable” means that the subject item is appropriate for use in a pharmaceutical product.

The compounds of Formula (I) of this invention are expected to be valuable as therapeutic agents. Accordingly, an embodiment of this invention includes a method of treating the various conditions in a patient (including mammals) which comprises administering to said patient a composition containing an amount of the compound of Formula (I) that is effective in treating the target condition.

An object of this invention is to provide methods for treating obesity and inducing weight loss in an individual by administration of a compound of the invention. The method of the invention comprises administering to an individual a therapeutically effective amount of at least one compound of the invention, or a prodrug thereof, which is sufficient to induce weight loss. The invention further comprises a method of preventing weight gain in an individual by administering an amount of at least one compound of the invention, or a prodrug thereof, which is sufficient to prevent weight gain.

The present invention also relates to the use of the compounds of this invention for the treatment of obesity-related diseases including associated dyslipidemia and other obesity- and overweight-related complications such as, for example, cholesterol gallstones, gallbladder disease, gout, cancer (e.g., colon, rectum, prostate, breast, ovary, endometrium, cervix, gallbladder, and bile duct), menstrual abnormalities, infertility, polycystic ovaries, osteoarthritis, and sleep apnea, as well as for a number of other pharmaceutical uses associated therewith, such as the regulation of appetite and food intake, dyslipidemia, hypertriglyceridemia, Syndrome X, type 2 diabetes (non-insulin-dependent diabetes), atherosclerotic diseases such as heart failure, hyperlipidemia, hypercholesteremia, low HDL levels, hypertension, cardiovascular disease (including atherosclerosis, coronary heart disease, coronary artery disease, and hypertension), cerebrovascular disease such as stroke, and peripheral vessel disease. The compounds of this invention may also be useful for treating physiological disorders related to, for example, regulation of insulin sensitivity, inflammatory response, plasma triglycerides, HDL, LDL and cholesterol levels and the like.

Compounds of Formula (I) may be administered alone or in combination with one or more additional therapeutic agents. Combination therapy includes administration of a single pharmaceutical dosage formulation which contains a compound of Formula (I) and one or more additional therapeutic agents, as well as administration of the compound of Formula (I) and each additional therapeutic agents in its own separate pharmaceutical dosage formulation. For example, a compound of Formula (I) and a therapeutic agent may be administered to the patient together in a single oral dosage composition such as a tablet or capsule, or each agent may be administered in separate oral dosage formulations.

Where separate dosage formulations are used, the compound of Formula (I) and one or more additional therapeutic agents may be administered at essentially the same time (e.g., concurrently) or at separately staggered times (e.g., sequentially).

For example, the compounds of Formula (I) may be used in combination with other therapies and drugs useful for the treatment of obesity. For example, anti-obesity drugs include β-3 adrenergic receptor agonists such as CL 316,243; cannabinoid (e.g., CB-1) antagonists such as Rimonabant; neuropeptide-Y receptor antagonists; neuropeptide Y5 inhibitors; apo-B/MTP inhibitors; 11β-hydroxy steroid dehydrogenase-1 inhibitors; peptide YY₃₋₃₆ or analogs thereof; MCR4 agonists; CCK-A agonists; monoamine reuptake inhibitors; sympathomimetic agents; dopainine agonists; melanocyte-stimulating hormone receptor analogs; melanin concentrating hormone antagonists; leptin; leptin analogs; leptin receptor agonists; galanin antagonists; lipase inhibitors; bombesin agonists; thyromimetic agents; dehydroepiandrosterone or analogs thereof; glucocorticoid receptor antagonists; orexin receptor antagonists; ciliary neurotrophic factor; ghrelin receptor antagonists; histamine-3-receptor antagonists; neuromedin U receptor agonists; appetite suppressants, such as, for example, sibutramine (Meridia); and lipase inhibitors, such as, for example, orlistat (Xenical). The compounds of the present invention may also be administered in combination with a drug compound that modulates digestion and/or metabolism such as drugs that modulate thermogenesis, lipolysis, gut motility, fat absorption, and satiety.

In addition, the compounds of Formula (I) may be administered in combination with one or more of the following agents for the treatment of diabetes or diabetes-related disorders including PPAR ligands (agonists, antagonists), insulin secretagogues, for example, sulfonylurea drugs and non-sulfonylurea secretagogues, α-glucosidase inhibitors, insulin sensitizers, hepatic glucose output lowering compounds, and insulin and insulin derivatives. Such therapies may be administered prior to, concurrently with, or following administration of the compounds of the invention. Insulin and insulin derivatives include both long and short acting forms and formulations of insulin. PPAR ligands may include agonists and/or antagonists of any of the PPAR receptors or combinations thereof. For example, PPAR ligands may include ligands of PPAR-αPPAR-γ, PPAR-δ or any combination of two or three of the receptors of PPAR. PPAR ligands include, for example, rosiglitazone, troglitazone, and pioglitazone. Sulfonylurea drugs include, for example, glyburide, glimepiride, chlorpropamide, tolbutamide, and glipizide. α-glucosidase inhibitors that may be useful in treating diabetes when administered with a compound of the invention include acarbose, miglitol, and voglibose. Insulin sensitizers that may be useful in treating diabetes include PPAR-γ agonists such as the glitazones (e.g., troglitazone, pioglitazone, englitazone, MCC-555, rosiglitazone, and the like) and other thiazolidinedione and non-thiazolidinedione compounds; biguanides such as metformin and phenformin; protein tyrosine phosphatase-1B (PTP-1B) inhibitors; dipeptidyl peptidase IV (DPP-IV) inhibitors, and 11beta-HSD inhibitors. Hepatic glucose output lowering compounds that may be useful in treating diabetes when administered with a compound of the invention include glucagon anatgonists and metformin, such as Glucophage and Glucophage XR. Insulin secretagogues that may be useful in treating diabetes when administered with a compound of the invention include sulfonylurea and non-sulfonylurea drugs: GLP-1, GIP, PACAP, secretin, and derivatives thereof; nateglinide, meglitinide, repaglinide, glibenclamide, glimepiride, chlorpropamide, glipizide. GLP-1 includes derivatives of GLP-1 with longer half-lives than native GLP-1, such as, for example, fatty-acid derivatized GLP-1 and exendin.

Compounds of the invention may also be used in methods of the invention in combination with drugs commonly used to treat lipid disorders in patients. Such drugs include, but are not limited to, HMG-CoA reductase inhibitors, nicotinic acid, fatty acid lowering compounds (e.g., acipimox); lipid lowering drugs (e.g., stanol esters, sterol glycosides such as tiqueside, and azetidinones such as ezetimibe), ACAT inhibitors (such as avasimibe), bile acid sequestrants, bile acid reuptake inhibitors, microsomal triglyceride transport inhibitors, and fibric acid derivatives. HMG-CoA reductase inhibitors include, for example, lovastatin, simvastatin, pravastatin, fluvastatin, atorvastatin, rivastatin, itavastatin, cerivastatin, and ZD-4522. Fibric acid derivatives include, for example, clofibrate, fenofibrate, bezafibrate, ciprofibrate, beclofibrate, etofibrate, and gemfibrozil. Sequestrants include, for example, cholestyramine, colestipol, and dialkylaminoalkyl derivatives of a cross-linked dextran.

Compounds of the invention may also be used in combination with anti-hypertensive drugs, such as, for example, β-blockers and ACE inhibitors. Examples of additional anti-hypertensive agents for use in combination with the compounds of the present invention include calcium channel blockers (L-type and T-type; e.g., diltiazem, verapamil, nifedipine, amlodipine and mybefradil), diuretics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, farosemide, musolimine, bumetanide, triamtrenene, amiloride, spironolactone), renin inhibitors, ACE inhibitors (e.g., captopril, zofenopril, fosinopril, enalapril, ceranopril, cilazopril, delapril, pentopril, quinapril, ramipril, lisinopril), AT-1 receptor antagonists (e.g., losartan, irbesartan, valsartan), ET receptor antagonists (e.g., sitaxsentan, atrsentan, neutral endopeptidase (NEP) inhibitors, vasopepsidase inhibitors (dual NEP-ACE inhibitors) (e.g., omapatrilat and gemopatrilat), and nitrates.

The compounds of Formula (I) may also be utilized, in free base form or in compositions, as well as in research and diagnostics or as analytical reference standards, and the like, which are well known in the art. Therefore, the present invention includes compositions which are comprised of an inert carrier and an effective amount of a compound of Formula (I) or a salt, or ester thereof. An inert carrier is any material which does not interact with the compound to be carried and which lends support, means of conveyance, bulk, traceable material, and the like to the compound to be carried. An effective amount of the compound is that amount which produces a result or exerts an influence on the particular procedure being performed.

It is anticipated that prodrug forms of the compounds of this invention will prove useful in certain circumstances, and such compounds are also intended to fall within the scope of the invention. Prodrug forms may have advantages over the parent compounds exemplified herein, in that they are better absorbed, better distributed, more readily penetrate the central nervous system, are more slowly metabolized or cleared, etc. Prodrug forms may also have formulation advantages in terms of crystallinity or water solubility. For example, compounds of the invention having one or more hydroxyl groups may be converted to esters or carbonates bearing one or more carboxyl, hydroxyl or amino groups, which are hydrolyzed at physiological pH values or are cleaved by endogenous esterases or lipases in vivo (see, e.g., U.S. Pat. Nos. 4,942,184; 4,960,790; 5,817,840; and 5,824,701, all of which are incorporated herein by reference in their entirety, and references therein).

### Pharmaceutical Compositions

Based on the above tests, or other well known assays used to determine the efficacy for treatment of conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered may generally range from about 0.001 mg/kg to about 200 mg/kg, and preferably from about 0.01 mg/kg to about 200 mg/kg body weight per day. A unit dosage may contain from about 0.05 mg to about 1500 mg of active ingredient, and may be administered one or more times per day. The daily dosage for administration by injection, including intravenous, intramuscular, subcutaneous, and parenteral injections, and use of infusion techniques may be from about 0.01 to about 200 mg/kg. The daily rectal dosage regimen may be from 0.01 to 200 mg/kg of total body weight. The transdermal concentration may be that required to maintain a daily dose of from 0.01 to 200 mg/kg.

Of course, the specific initial and continuing dosage regimen for each patient will vary according to the nature and severity of the condition as determined by the attending diagnostician, the activity of the specific compound employed, the age of the patient, the diet of the patient, time of administration, route of administration, rate of excretion of the drug, drug combinations, and the like. The desired mode of treatment and number of doses of a compound of the present invention or a pharmaceutically acceptable salt thereof may be ascertained by those skilled in the art using conventional treatment tests.

The compounds of this invention may be utilized to achieve the desired pharmacological effect by administration to a subject in need thereof in an appropriately formulated pharmaceutical composition. A subject, for example, may be a mammal, including a human, in need of treatment for a particular condition or disease. Therefore, the present invention includes pharmaceutical compositions which are comprised of a pharmaceutically acceptable carrier and a pharmaceutically effective amount of a compound identified by the methods described herein, or a pharmaceutically acceptable salt or ester thereof. A pharmaceutically acceptable carrier is any carrier which is relatively non-toxic and innocuous to a patient at concentrations consistent with effective activity of the active ingredient so that any side effects ascribable to the carrier do not vitiate the beneficial effects of the active ingredient. A pharmaceutically effective amount of a compound is that amount which produces a result or exerts an influence on the particular condition being treated. The compounds identified by the methods described herein may be administered with a pharmaceutically-acceptable carrier using any effective conventional dosage unit forms, including, for example, immediate and timed release preparations, orally, parenterally, topically, or the like.

For oral administration, the compounds may be formulated into solid or liquid preparations such as, for example, capsules, pills, tablets, troches, lozenges, melts, powders, solutions, suspensions, or emulsions, and may be prepared according to methods known to the art for the manufacture of pharmaceutical compositions. The solid unit dosage forms may be a capsule which can be of the ordinary hard- or soft-shelled gelatin type containing, for example, surfactants, lubricants, and inert fillers such as lactose, sucrose, calcium phosphate, and corn starch.

In another embodiment, the compounds of this invention may be tableted with conventional tablet bases such as lactose, sucrose, and cornstarch in combination with binders such as acacia, cornstarch, or gelatin; disintegrating agents intended to assist the break-up and dissolution of the tablet following administration such as potato starch, alginic acid, corn starch, and guar gum; lubricants intended to improve the flow of tablet granulation and to prevent the adhesion of tablet material to the surfaces of the tablet dies and punches, for example, talc, stearic acid, or magnesium, calcium or zinc stearate; dyes; coloring agents; and flavoring agents intended to enhance the aesthetic qualities of the tablets and make them more acceptable to the patient. Suitable excipients for use in oral liquid dosage forms include diluents such as water and alcohols, for example, ethanol, benzyl alcohol, and polyethylene alcohols, either with or without the addition of a pharmaceutically acceptable surfactant, suspending agent, or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent, and one or more preservatives. Suitable dispersing or wetting agents and suspending agents are exemplified by those already mentioned above. Additional excipients, for example, those sweetening, flavoring and coloring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived from fatty acids and hexitol anhydrides, for example, sorbitan monooleate, and (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil, or coconut oil; or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol, or sucrose. Such formulations may also contain a demulcent, and preservative, flavoring and coloring agents.

The compounds of this invention may also be administered parenterally, that is, subcutaneously, intravenously, intramuscularly, or interperitoneally, as injectable dosages of the compound in a physiologically acceptable diluent with a pharmaceutical carrier which may be a sterile liquid or mixture of liquids such as water, saline, aqueous dextrose and related sugar solutions; an alcohol such as ethanol, isopropanol, or hexadecyl alcohol; glycols such as propylene glycol or polyethylene glycol; glycerol ketals such as 2,2-dimethyl-1,1-dioxolane-4-methanol, ethers such as poly(ethyleneglycol) 400; an oil; a fatty acid; a fatty acid ester or glyceride; or an acetylated fatty acid glyceride with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum, and mineral oil. Suitable fatty acids include oleic acid, stearic acid, and isostearic acid. Suitable fatty acid esters are, for example, ethyl oleate and isopropyl myristate. Suitable soaps include fatty alkali metal, ammonium, and triethanolamine salts and suitable detergents include cationic detergents, for example, dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; nonionic detergents, for example, fatty amine oxides, fatty acid alkanolamides, and polyoxyethylenepolypropylene copolymers; and amphoteric detergents, for example, alkyl-beta-aminopropionates, and 2-alkylimidazoline quaternary ammonium salts, as well as mixtures.

The parenteral compositions of this invention may typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadecaethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, and isotonic sodium chloride solution. In addition, sterile fixed oils are conventionally employed as solvents or suspending media. For this purpose, any bland, fixed oil may be employed including synthetic mono or diglycerides. In addition, fatty acids such as oleic acid may be used in the preparation of injectables.

A composition of the invention may also be administered in the form of suppositories for rectal administration of the drug. These compositions may be prepared by mixing the drug with a suitable non-irritation excipient which is solid at ordinary temperatures but liquid at the rectal temperature and will therefore melt in the rectum to release the drug. Such material are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, incorporated herein by reference). Such patches may be constructed for continuous, pulsatile, or on demand delivery of pharmaceutical agents.

It may be desirable or necessary to introduce the pharmaceutical composition to the patient via a mechanical delivery device. The construction and use of mechanical delivery devices for the delivery of pharmaceutical agents is well known in the art. For example, direct techniques for administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, incorporated herein by reference.

The compositions of the invention may also contain other conventional pharmaceutically acceptable compounding ingredients, generally referred to as carriers or diluents, as necessary or desired. Any of the compositions of this invention may be preserved by the addition of an antioxidant such as ascorbic acid or by other suitable preservatives. Conventional procedures for preparing such compositions in appropriate dosage forms can be utilized.

Commonly used pharmaceutical ingredients which may be used as appropriate to formulate the composition for its intended route of administration include: acidifying agents, for example, but are not limited to, acetic acid, citric acid, fumaric acid, hydrochloric acid, nitric acid; and alkalinizing agents such as, but are not limited to, ammonia solution, ammonium carbonate, diethanolamine, monoethanolamine, potassium hydroxide, sodium borate, sodium carbonate, sodium hydroxide, triethanolamine, trolamine.

Other pharmaceutical ingredients include, for example, but are not limited to, adsorbents (e.g., powdered cellulose and activated charcoal); aerosol propellants (e.g., carbon dioxide, CCl₂F₂, F₂ClC-CClF₂ and CClF₃); air displacement agents (e.g., nitrogen and argon); antifungal preservatives (e.g., benzoic acid, butylparaben, ethylparaben, methylparaben, propylparaben, sodium benzoate); antimicrobial preservatives (e.g., benzalkonium chloride, benzethonium chloride, benzyl alcohol, cetylpyridinium chloride, chlorobutanol, phenol, phenylethyl alcohol, phenylmercuric nitrate and thimerosal); antioxidants (e.g., ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite); binding materials (e.g., block polymers, natural and synthetic rubber, polyacrylates, polyurethanes, silicones and styrene-butadiene copolymers); buffering agents (e.g., potassium metaphosphate, potassium phosphate monobasic, sodium acetate, sodium citrate anhydrous and sodium citrate dihydrate); carrying agents (e.g., acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection); chelating agents (e.g., edetate disodium and edetic acid); colorants (e.g., FD&C Red No. 3, FD&C Red No. 20, FD&C Yellow No. 6, FD&C Blue No. 2, D&C Green No. 5, D&C Orange No. 5, D&C Red No. 8, caramel and ferric oxide red); clarifying agents (e.g., bentonite); emulsifying agents (but are not limited to, acacia, cetomacrogol, cetyl alcohol, glyceryl monostearate, lecithin, sorbitan monooleate, polyethylene 50 stearate); encapsulating agents (e.g., gelatin and cellulose acetate phthalate); flavorants (e.g., anise oil, cinnamon oil, cocoa, menthol, orange oil, peppermint oil and vanillin); humectants (e.g., glycerin, propylene glycol and sorbitol); levigating agents (e.g., mineral oil and glycerin); oils (e.g., arachis oil, mineral oil, olive oil, peanut oil, sesame oil and vegetable oil); ointment bases (e.g., lanolin, hydrophilic ointment, polyethylene glycol ointment, petrolatum, hydrophilic petrolatum, white ointment, yellow ointment, and rose water ointment); penetration enhancers (transdermal delivery) (e.g., monohydroxy or polyhydroxy alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas); plasticizers (e.g., diethyl phthalate and glycerin); solvents (e.g., alcohol, corn oil, cottonseed oil, glycerin, isopropyl alcohol, mineral oil, oleic acid, peanut oil, purified water, water for injection, sterile water for injection and sterile water for irrigation); stiffening agents (e.g., cetyl alcohol, cetyl esters wax, microcrystalline wax, paraffin, stearyl alcohol, white wax and yellow wax); suppository bases (e.g., cocoa butter and polyethylene glycols (mixtures)); surfactants (e.g., benzalkonium chloride, nonoxynol 10, oxtoxynol 9, polysorbate 80, sodium lauryl sulfate and sorbitan monopalmitate); suspending agents (e.g., agar, bentonite, carbomers, carboxymethylcellulose sodium, hydroxyethyl cellulose, hydroxypropyl cellulose, hydroxypropyl methylcellulose, kaolin, methylcellulose, tragacanth and veegum); sweetening e.g., aspartame, dextrose, glycerin, mannitol, propylene glycol, saccharin sodium, sorbitol and sucrose); tablet anti-adherents (e.g., magnesium stearate and talc); tablet binders (e.g., acacia, alginic acid, carboxymethylcellulose sodium, compressible sugar, ethylcellulose, gelatin, liquid glucose, methylcellulose, povidone and pregelatinized starch); tablet and capsule diluents (e.g., dibasic calcium phosphate, kaolin, lactose, mannitol, microcrystalline cellulose, powdered cellulose, precipitated calcium carbonate, sodium carbonate, sodium phosphate, sorbitol and starch); tablet coating agents (e.g., liquid glucose, hydroxyethyl cellulose, hydroxypropyl cellulose, hydroxypropyl methylcellulose, methylcellulose, ethylcellulose, cellulose acetate phthalate and shellac); tablet direct compression excipients (e.g., dibasic calcium phosphate); tablet disintegrants (e.g., alginic acid, carboxymethylcellulose calcium, microcrystalline cellulose, polacrilin potassium, sodium alginate, sodium starch glycollate and starch); tablet glidants (e.g., colloidal silica, corn starch and talc); tablet lubricants (e.g., calcium stearate, magnesium stearate, mineral oil, stearic acid and zinc stearate); tablet/capsule opaquants (e.g., titanium dioxide); tablet polishing agents (e.g., carnuba wax and white wax); thickening agents (e.g., beeswax, cetyl alcohol and paraffin); tonicity agents (e.g., dextrose and sodium chloride); viscosity increasing agents (e.g., alginic acid, bentonite, carbomers, carboxymethylcellulose sodium, methylcellulose, povidone, sodium alginate and tragacanth); and wetting agents (e.g., heptadecaethylene oxycetanol, lecithins, polyethylene sorbitol monooleate, polyoxyethylene sorbitol monooleate, and polyoxyethylene stearate).

The compounds identified by the methods described herein may be administered as the sole pharmaceutical agent or in combination with one or more other pharmaceutical agents where the combination causes no unacceptable adverse effects. For example, the compounds of this invention can be combined with known anti-obesity, or with known antidiabetic or other indication agents, and the like, as well as with admixtures and combinations thereof.

The compounds identified by the methods described herein may also be utilized, in free base form or in compositions, in research and diagnostics, or as analytical reference standards, and the like. Therefore, the present invention includes compositions which are comprised of an inert carrier and an effective amount of a compound identified by the methods described herein, or a salt or ester thereof. An inert carrier is any material which does not interact with the compound to be carried and which lends support, means of conveyance, bulk, traceable material, and the like to the compound to be carried. An effective amount of compound is that amount which produces a result or exerts an influence on the particular procedure being performed.

Formulations suitable for subcutaneous, intravenous, intramuscular, and the like; suitable pharmaceutical carriers; and techniques for formulation and administration may be prepared by any of the methods well known in the art (see, e.g., Remington's Pharmaceutical Sciences, Mack Publishing Co., Easton, Pa., 20^{th} edition, 2000).

### Biological Activity of the Compounds

In order that this invention may be better understood, the following examples are set forth. These examples are for the purpose of illustration only, and are not to be construed as limiting the scope of the invention in any manner. All publications mentioned herein are incorporated by reference in their entirety.

Demonstration of the activity of the compounds of the present invention may be accomplished through in vitro, ex vivo, and in vivo assays that are well known in the art. For example, to demonstrate the efficacy of a pharmaceutical agent for the treatment of obesity and related disorders, the following assays may be used.

### Evaluation of Compound Effect on the Inhibition of DGAT-1 Enzyme Activity

The human DGAT-1 gene (see, e.g., U.S. Pat. No. 6,100,077) was isolated from a human cDNA library by PCR. Recombinant AcNPV baculovirus was constructed in which the gene for occlusion body forming protein polyhedrin was replaced with the DGAT-1 gene. The DGAT-1 gene sequence was inserted into the AcNPV genome 3′ to the polyhedrin promoter sequence placing DGAT-1 under the transcriptional control of the polyhedrin promoter. Spodoptera frugiperda-derived Sf9 insect cells were infected with DGAT-1-containing recombinant baculovirus at the multiplicity of infection of 5 and harvested 48 h post-infection. DGAT-1-expressing insect cells were homogenized in 10 mM Tris, 250 mM sucrose, pH 7.5 at the concentration of 100 mg of wet cell biomass per mL. The homogenate was centrifuged at 25,000 g for 30 minutes. The 25,000 g pellet was discarded and the supernatant was centrifuged at 100,000 g for 1 h. The 100,000 g supernatant was discarded and the 100,000 g DGAT-1-containing membrane pellet was re-suspended in 10 mM Tris, 50% (v/v) glycerol pH 7.5.

DGAT-1 enzyme activity was determined by a phase partitioning protocol. Specifically, DGAT-1 containing membranes were incubated in 20 μM didecanoyl glycerol, 5 μM ¹⁴C-decanoyl-CoA, 2 mM MgCl₂, 0.04% BSA, 20 mM HEPES, pH 7.5 buffer in the presence of varying concentrations of inhibitors. Assays were performed in 100 μl volumes in 96-well microtiter plates 0.5 μg total membrane protein per well. The assay was initiated by substrate and mixed gently for 1 h at ambient temperature. Activity was quenched by the addition of 25 μl of 0.1% phosphoric acid solution. Selective extraction of the hydrophobic tridecanolyglycerol product was accomplished by the addition of 150 μl phase partitioning scintillation fluid Microscint® (Packard, Inc.) and vigorous mixing for 30 minutes. Quantification of the product was accomplished by a MicroBeta® scintillation counter (Wallac, Inc.) after settling for approximately 16 h at ambient temperatures.

### Evaluation of Compound Effect on the Inhibition of Cellular Triglyceride Deposition

The cell-based assay for DGAT-1 was conducted with human colorectal adenocarcinoma cells HT-29 (HTB-38, ATCC). HT-29 cells were grown in 75 cm² plate until ˜90% confluent in DMEM media with 10% FBS, PSF, glutamine, and 10 mM acetate. Cells were then re-plated in 24-well plates to give 1:1.2 dilution and grown approximately 16 h. Triacylglyceride formation was stimulated by the addition of lauric acid to 0.01% final concentration in the presence of varying concentrations of inhibitors. After 6 h, cells were released from the plate by trypsin, collected by centrifugation, re-suspended in water, transferred to glass HPLC, frozen at −70° C., and lyophilized. Freeze dried cell pellets were re-suspended in 150 μl HPLC grade tetrahydrofuran and sealed in the vials. Vials were sonicated for 30 minutes with heating in a sonicating water bath (Fisher, Inc.). Cellular triacylglycerides were quantified by HPLC (HP1100, Agilent, Inc.) utilizing evaporative light-scattering detection (PL-ELS 1000, Polymer Labs, Inc.). Chromatographic separation was accomplished by 30 to 100% B buffer in 4 minutes followed by 3 minutes at 100% B buffer using a PLRP S 100 column (5 micron, 150×4.6 mm, Polymer Labs, Inc.) at 50° C. (A: 50% acetonitrile, 2.5% methanol, B: 100% tetrahydrofuran). Sample injections were 20 μl and the detector was set at 0.4 SLM, 40° C. nebulizer and 80° C. evaporator. Non-polar fatty acids and glycerol lipids were identified and quantified by using commercially available standards.

### Evaluation of Compound Efficacy on the Reduction of Body Weight in Diet-Induced Obese Mice

The purpose of this protocol is to determine the effect of chronic administration of a compound on the body weight of mice made obese by exposure to a 45% kcal/g high fat diet for more than 10 weeks. The body weight of mice selected for these studies was higher than three standard deviations from the weight of a control group of mice fed standard low fat (5-6% fat) mouse chow. Diet-induced obese (DIO) animals have been used frequently in the determination of compound efficacy in the reduction of body weight (see, e.g., Brown, et al., Brit. J. Pharmacol. 132:1898-1904, 2001; Guerre-Millo, et al., J. Biol. Chem. 275(22):16638-42, 2000; Han, et al., Intl. J. Obesity and Related Metabolic Disorders 23(2):174-79, 1999; Surwit, et al., Endocrinol. 141(10):3630-37, 2000).

This animal model has been successfully used in the identification and characterization of the efficacy profile of compounds that are or have been used in the management of body weight in obese humans (see, e.g., Brown, et al., 2001; Guerre-Millo, et al., 2000; Han, et al., 1999).

A typical study included 60-80 male C57b1/J6 mice (n=10/treatment group) with an average body weight of approximately 45 g. Mice were kept in standard animal rooms under controlled temperature and humidity and a 12 hour/12 hour light/dark cycle. Water and food were continuously available. Mice were individually housed. Animals were sham dosed with study vehicle for at least four days before the recording of two-day baseline measurements of body weight and 24 hour food and water consumption. Mice were assigned to one of 6-8 treatment groups based upon their body weight on baseline. The groups were set up so that the mean and standard error of the mean of body weight were similar.

Animals were orally gavaged (5 mL/kg) daily before the dark phase of the light/dark cycle for a pre-determined number of days (typically 8-14 days) with their assigned dose/compound. Body weight, and food and water consumption were measured. Data was analyzed using appropriate statistics following the research design. On the final day, animals were euthanized using CO₂ inhalation.

Compounds were typically dosed at 5 or 10 mg/kg p.o. q.d. as a suspension formulation in 50:50 PEG/water, or p.o. b.i.d. as a suspension formulation in 0.5% methylcellulose, and compounds were considered to be active if a statistically significant reduction in body weight was observed for the treated animals after a treatment period of at least seven days, relative to vehicle-treated control animals.

The structures, materials, compositions, and methods described herein are intended to be representative examples of the invention, and it will be understood that the scope of the invention is not limited by the scope of the examples. Those skilled in the art will recognize that the invention may be practiced with variations on the disclosed structures, materials, compositions and methods, and such variations are regarded as within the ambit of the invention.

LENGTHY TABLES
The patent application contains a lengthy table section. A copy of the table is available in electronic form from the USPTO web site (http://seqdata.uspto.gov/?pageRequest=docDetail&DocID=US20090215780A1). An electronic copy of the table will also be available from the USPTO upon request and payment of the fee set forth in 37 CFR 1.19(b)(3).

What is claimed:

1. A compound of Formula (I) 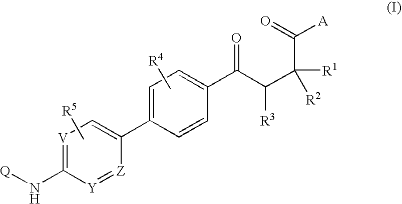
wherein
R² and R³ are both hydrogen, and R¹ is hydrogen, (C₁-C₆)alkyl, (C₁-C₆)alkoxy-(C₂-C₆)alkyl, phenoxy-(C₂-C₆)alkyl, 1-methyl-1H-indol-3-yl, bis[(C₁-C₆)alkyl]amino-(C₂-C₆)alkyl, 1-piperidinyl-(C₂-C₆)alkyl, 1-pyrrolidinyl-(C₂-C₆)alkyl, or 1-morpholinyl-(C₂-C₆)alkyl;
or
R¹ is R⁶(CH₂)ₘ, where m is 0 to 3, and R⁶ is phenyl optionally substituted with one or more halogen, hydroxy, (C₁-C₆)alkyl, (C₁-C₆)alkoxy, trifluoromethyl, cyano, or nitro;
or
R⁶ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
R³ is hydrogen, and R¹ and R² are identical and are each selected from (C₁-C₆)alkyl;
or
R³ is hydrogen, and R¹ and R² together with the carbon atom to which they are attached, form a three-to five-membered carbocyclic ring, or form a six-membered ring represented by 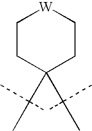
wherein W is CH₂, C(CH₃)₂, O, NH, N(CH₃), S, or SO₂;
or
R¹ is hydrogen, and R² and R³ together with the two carbon atoms to which they are attached, form a three- to six-membered carbocyclic ring;
R⁴ and R⁵ are independently selected from hydrogen, hydroxy, halo, (C₁-C₆)alkyl, (C₁-C₆)alkoxy, trifluoromethyl, and cyano;
Q is R⁷—C(O)—, where R⁷ is (C₁-C₆)alkyl optionally substituted with one or more hydroxy, (C₁-C₆)alkoxy, bis[(C₁-C₆)alkyl)amino, or fluoro;
or
R⁷ is R⁸(CH₂)ₙ, where n is 0 to 3, and R⁸ is phenyl optionally substituted with one or more halogen, hydroxy, (C₁-C₆)alkyl, (C₁-C₆)alkoxy, trifluoromethyl, cyano, or nitro;
or
R⁸ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
or
R⁷ is R¹⁰C(R⁹)₂, where R⁹ is methyl or ethyl,
or
C(R⁹)₂ is a 1,1-cyclopropyl, 1,1-cyclobutyl, 1,1-cyclopentyl, or 1,1-cyclohexyl ring;
R¹⁰ is phenyl optionally substituted with one or more halogen, hydroxy, (C₁-C₆)alkyl, (C₁-C₆)alkoxy, trifluoromethyl, cyano, or nitro;
or
R¹⁰ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
R⁷ is a fragment group selected from 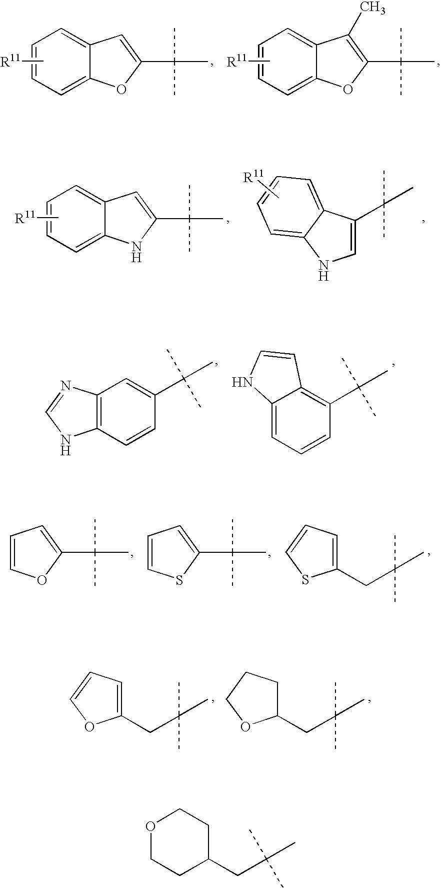
wherein R¹¹ is one or more substituents selected from halogen, hydroxy, (C₁-C₆)alkyl, (C₁-C₆)alkoxy, trifluoromethyl, cyano, and nitro;
or
Q is R¹³—N(R²)—C(O)—, where R¹² is hydrogen or (C₁-C₆)alkyl, and
R¹³ is (C₁-C₆)alkyl optionally substituted with one or more hydroxy, (C₁-C₆)alkoxy, bis[(C₁-C₆)alkyl)amino, or fluoro;
or
R¹³ is R¹⁴(CH₂)ₚ, where p is 0 to 3, and R¹⁴ is phenyl optionally substituted with one or more halogen, hydroxy, (C₁-C₆)alkyl, (C₁-C₆)alkoxy, trifluoromethyl, cyano, or nitro;
or
R¹⁴ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
or
R¹² and R¹³ and the nitrogen atom to which they are attached form a ring fragment, selected from 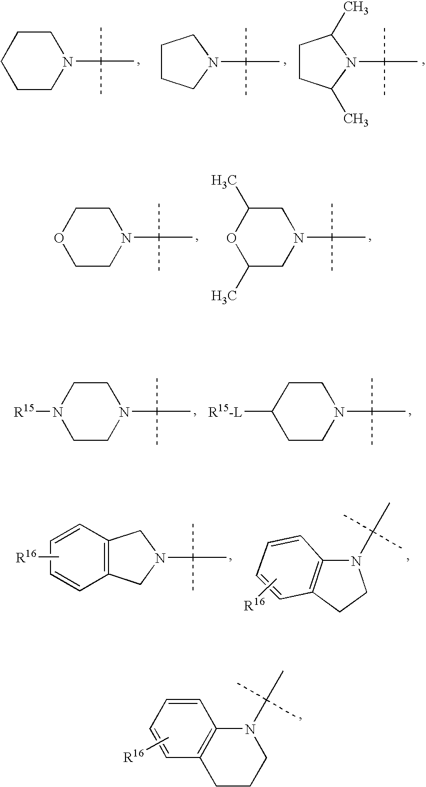
wherein L is O, C(O), or a bond;
R¹⁵ is (C₁-C₆)alkyl;
or
R¹⁵ is R¹⁷(CH₂)_{q}, where q is 0 or 1, and R¹⁷ is phenyl optionally substituted with one or more halogen, hydroxy, (C₁-C₆)alkyl, (C₁-C₆)alkoxy, trifluoromethyl, cyano, or nitro;
or
R¹⁷ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
R¹⁶ is one or more substituents selected from halogen, hydroxy, (C₁-C₆)alkyl, (C₁-C₆)alkoxy, trifluoromethyl, cyano, and nitro;
or
Q is R¹⁸—S(O)₂—, where R¹⁸ is (C₁-C₆)alkyl or benzyl;
or
R¹⁸ is phenyl optionally substituted with one or more halogen, hydroxy, (C₁-C₆)alkyl, (C₁-C₆)alkoxy, trifluoromethyl, cyano, or nitro;
A is OH, or NHS(O)₂—R¹⁹
where R¹⁹ is (C₁-C₆)alkyl, trifluoromethyl, benzyl;
or
R¹⁹ is R²⁰(CH₂)ₜ, where t is 0 or 1, and R²⁰ is phenyl optionally substituted with one or more halogen, hydroxy, (C₁-C₆)alkyl, (C₁-C₆)alkoxy, trifluoromethyl, cyano, or nitro;
or
R¹⁹ is a fragment group selected from 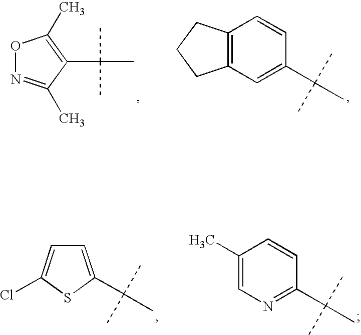
V, Y, and Z are all carbon; or
V and Y are carbon and Z is nitrogen; or
V and Z are carbon and Y is nitrogen; or
Z is carbon and V and Y are both nitrogen;
and pharmaceutically acceptable salts and esters thereof,
with the proviso that Formula (I) is not 4-[4′-(acetylamino)-3′-bromobiphenyl-4-yl]-4-oxobutanoic acid, 4-[4′-(acetylamino)biphenyl-4-yl]-4-oxo-2-(2-phenylethyl)butanoic acid, 4-{4′-[(3,3-dimethyl-butanoyl)amino]biphenyl-4-yl})-oxo-2-(2-phenylethyl)butanoic acid, or 4-oxo-4-[4′-(pentanoyl-amino)biphenyl-4-yl]-2-(2-phenylethyl)butanoic acid.

2. The compound of claim 1, wherein
wherein
R² and R³ are both hydrogen, and R¹ is hydrogen, (C₁-C₆)alkyl, (C₁-C₆)alkoxy-(2-C₆)alkyl, phenoxy-(C₂-C₆)alkyl, 1-methyl-1H-indol-3-yl, bis[(C₁-C₆)alkyl]amino-(C₂-C₆)alkyl, 1-piperidinyl-(C₂-C₆)alkyl, 1-pyrrolidinyl-(C₁-C₆)alkyl, or 1-morpholinyl-(C₂-C₆)alkyl;
or
R¹ is R⁶(CH₂)ₘ, where m is 0 to 3, and R⁶ is phenyl optionally substituted with one or more halogen, hydroxy, (C₁-C₆)alkyl, (C₁-C₆)alkoxy, trifluoromethyl, cyano, or nitro;
or
R⁶ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro.

3. The compound of claim 1, wherein
R¹ is hydrogen, and R² and R³ together with the two carbon atoms to which they are attached, form a three- to six-membered carbocyclic ring.

4. The compound of claim 1, wherein A is OH.

5. The compound of claim 1, wherein A is NHS(O)₂—R¹⁹
where R¹⁹ is (C₁-C₆)alkyl, trifluoromethyl, benzyl;
or
R¹⁹ is R²⁰(CH₂)ₜ, where t is 0 or 1, and R²⁰ is phenyl optionally substituted with one or more halogen, hydroxy, (C₁-C₆)alkyl, (C₁-C₆)alkoxy, trifluoromethyl, cyano, or nitro;
or
R¹⁹ is a fragment group selected from

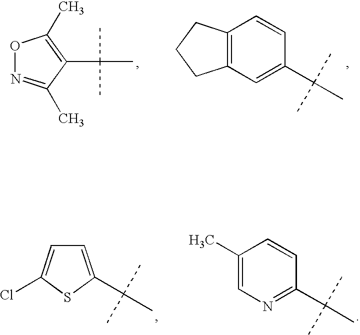

6. The compound of claim 1, wherein
V and Y are carbon and Z is nitrogen.

7. The compound of claim 1, wherein
V and Z are carbon and Y is nitrogen.

8. The compound of claim 1, wherein
V and Y are nitrogen and Z is carbon.

9. The compound of claim 1, wherein
R² and R³ are both hydrogen;
and
R¹ is R⁶(CH₂)ₘ, where m is 0 to 3, and R⁶ is phenyl optionally substituted with one or more halogen, hydroxy, (C₁-C₆)alkyl, (C₁-C₆)alkoxy, trifluoromethyl cyano, or nitro;
or
R⁶ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
Q is R⁷—C(O)—, where R⁷ is (C₁-C₆)alkyl optionally substituted with one or more hydroxy, (C₁-C₆)alkoxy, bis[(C₁-C₆)alkyl)amino, or fluoro;
or
R⁷ is R⁸(CH₂)ₙ, where n is 0 to 3, and R⁸ is phenyl optionally substituted with one or more halogen, hydroxy, (C₁-C₆)alkyl, (C₁-C₆)alkoxy, trifluoromethyl, cyano, or nitro;
or
R⁸ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
or
R⁷ is ROC(R⁹)₂, where R⁹ is methyl or ethyl,
or
C(R⁹)₂ is a 1,1-cyclopropyl, 1,1-cyclobutyl, 1,1-cyclopentyl, or 1,1-cyclohexyl ring;
R¹⁰ is phenyl optionally substituted with one or more halogen, hydroxy, (C₁-C₆)alkyl, (C₁-C₆)alkoxy, trifluoromethyl, cyano, or nitro;
or
R¹⁰ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
R⁷ is a fragment group selected from 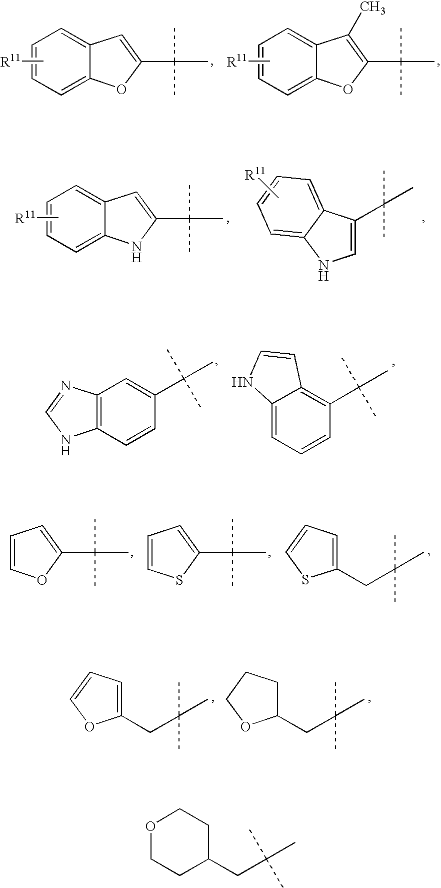
wherein R¹¹ is one or more substituents selected from halogen, hydroxy, (C₁-C₆)alkyl, (C₁-C₆)alkoxy, trifluoromethyl, cyano, and nitro;
and
A is OH.

10. The compound of claim 1, wherein
R³ is hydrogen, and R¹ and R² together with the carbon atom to which they are attached, form a three-to five-membered carbocyclic ring, or form a six-membered ring represented by 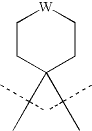
wherein W is CH₂, C(CH₃)₂, O, NH, N(CH₃), S, or SO₂;
or
R¹ is hydrogen, and R² and R³ together with the two carbon atoms to which they are attached, form a three- to six-membered carbocyclic ring;
Q is R⁷—C(O)—, where R⁷ is (C₁-C₆)alkyl optionally substituted with one or more hydroxy, (C₁-C₆)alkoxy, bis[(C₁-C₆)alkyl)amino, or fluoro;
or
R⁷ is R⁸(CH₂)ₙ, where n is 0 to 3, and R⁸ is phenyl optionally substituted with one or more halogen, hydroxy, (C₁-C₆)alkyl, (C₁-C₆)alkoxy, trifluoromethyl, cyano, or nitro;
or
R⁸ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
or
R⁷ is R¹⁰C(R⁹)₂, where R⁹ is methyl or ethyl,
or
C(R⁹)₂ is a 1,1-cyclopropyl, 1,1-cyclobutyl, 1,1-cyclopentyl, or 1,1-cyclohexyl ring;
R¹⁰ is phenyl optionally substituted with one or more halogen, hydroxy, (C₁-C₆)alkyl, (C₁-C₆)alkoxy, trifluoromethyl, cyano, or nitro;
or
R¹⁰ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
R⁷ is a fragment group selected from 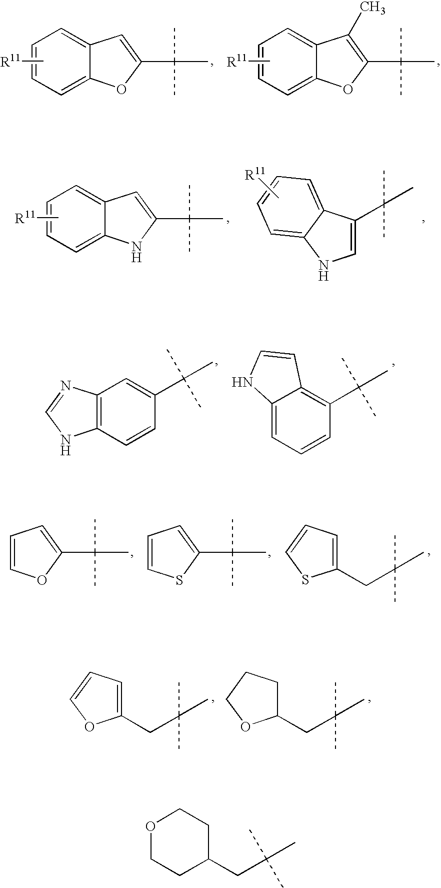
wherein R¹¹ is one or more substituents selected from halogen, hydroxy, (C₁-C₆)alkyl, (C₁-C₆)alkoxy, trifluoromethyl, cyano, and nitro;
A is OH.

11. A compound of claim 1, wherein
wherein
R² and R³ are both hydrogen, and R¹ is hydrogen, (C₁-C₆)alkyl, (C₁-C₆)alkoxy-(C₂-C₆)alkyl, phenoxy-(C₂-C₆)alkyl, 1-methyl-1H-indol-3-yl, bis[(C₁-C₆)alkyl]amino-(C₁-C₆)alkyl, 1-piperidinyl-(C₂-C₆)alkyl, 1-pyrrolidinyl-(C₂-C₆)alkyl, or 1-morpholinyl-(C₂-C₆)alkyl;
or
R¹ is R⁶(CH₂)ₘ where m is 0 to 3, and R⁶ is phenyl optionally substituted with one or more halogen, hydroxy, (C₁-C₆)alkyl, (C₁-C₆)alkoxy, trifluoromethyl, cyano, or nitro;
or
R⁶ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
Q is R¹³—N(R¹²)—C(O)—, where R¹² is hydrogen or (C₁-C₆)alkyl, and
R¹³ is (C₁-C₆)alkyl optionally substituted with one or more hydroxy, (C₁-C₆)alkoxy, bis[(C₁-C₆)alkyl)amino, or fluoro;
or
R¹³ is R¹⁴(CH₂)ₚ, where p is 0 to 3, and R¹⁴ is phenyl optionally substituted with one or more halogen, hydroxy, (C₁-C₆)alkyl, (C₁-C₆)alkoxy, trifluoromethyl, cyano, or nitro;
or
R¹⁴ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
or
R¹² and R¹³ and the nitrogen atom to which they are attached form a ring fragment, selected from 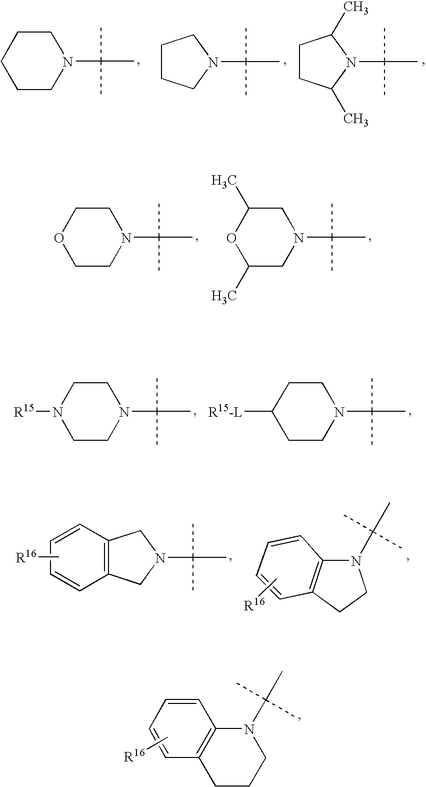
wherein L is O, C(O), or a bond;
R¹⁵ is (C₁-C₆)alkyl;
or
R¹⁵ is R¹⁷(CH₂)_{q}, where q is 0 or 1, and R¹⁷ is phenyl optionally substituted with one or more halogen, hydroxy, (C₁-C₆)alkyl, (C₁-C₆)alkoxy, trifluoromethyl, cyano, or nitro;
or
R¹⁷ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
R¹⁶ is one or more substituents selected from halogen, hydroxy, (C₁-C₆)alkyl, (C₁-C₆)alkoxy, trifluoromethyl, cyano, and nitro;
and
A is OH.

12. The compound of claim 1, wherein
R³ is hydrogen, and R¹ and R² together with the carbon atom to which they are attached, form a three to five-membered carbocyclic ring, or form a six-membered ring represented by 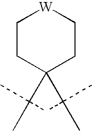
wherein W is CH₂, C(CH₃)₂, O, NH, N(CH₃), S, or SO₂;
or
R¹ is hydrogen, and R² and R³ together with the two carbon atoms to which they are attached, form a three- to six-membered carbocyclic ring;
Q is R¹³—N(R²)—C(O)—, where R¹² is hydrogen or (C₁-C₆)alkyl, and
R¹³ is (C₁-C₆)alkyl optionally substituted with one or more hydroxy, (C₁-C₆)alkoxy, bis[(C₁-C₆)alkyl)amino, or fluoro;
or
R¹³ is R¹⁴(CH₂)ₚ, where p is 0 to 3, and R¹⁴ is phenyl optionally substituted with one or more halogen, hydroxy, (C₁-C₆)alkyl, (C₁-C₆)alkoxy, trifluoromethyl, cyano, or nitro;
or
R¹⁴ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
or
R¹² and R¹³ and the nitrogen atom to which they are attached form a ring fragment, selected from 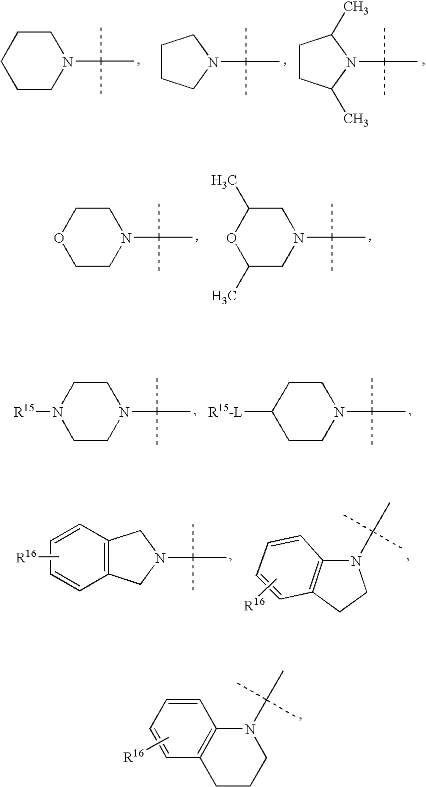
wherein L is O, C(O), or a bond;
R¹⁵ is (C₁-C₆)alkyl;
or
R¹⁵ is R¹⁷(CH₂)_{q}, where q is 0 or 1, and R¹⁷ is phenyl optionally substituted with one or more halogen, hydroxy, (C₁-C₆)alkyl, (C₁-C₆)alkoxy, trifluoromethyl, cyano, or nitro;
or
R¹⁷ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
R¹⁶ is one or more substituents selected from halogen, hydroxy, (C₁-C₆)alkyl, (C₁-C₆)alkoxy, trifluoromethyl, cyano, and nitro;
and
A is OH.

13. The compound of claim 1,
wherein
R³ is hydrogen, and R¹ and R² are both methyl;
or
R¹ is hydrogen, and R² and R³ together with the two carbon atoms to which they are attached, form a three- to six-membered carbocyclic ring;
R⁴ and R⁵ are independently selected from hydrogen and halo;
Q is R⁷—C(O)—,
wherein
R⁷ is R⁸(CH₂)ₙ, wherein n is 0 to 3, and R¹ is phenyl optionally substituted with one or more halogen, hydroxy, (C₁-C₆)alkyl, (C₁-C₆)alkoxy, trifluoromethyl, cyano, or nitro,
or
R⁸ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
A is OH; and
V, Y, and Z are all carbon.

14. The compound of claim 1,
wherein
R³ is hydrogen, and R¹ and R² are both methyl;
or
R¹ is hydrogen, and R² and R³ together with the two carbon atoms to which they are attached, form a three- to six-membered carbocyclic ring;
R⁴ and R⁵ are independently selected from hydrogen and halo;
Q is R¹³—N(R¹²)—(O)—,
wherein
R¹² is hydrogen; and
R¹³ is R¹⁴(CH₂)ₚ, wherein p is 0 to 3, and R¹⁴ is phenyl optionally substituted with one or more halogen, hydroxy, (C₁-C₆)alkyl, (C₁-C₆)alkoxy, triiluoromethyl, cyano, or nitro,
or
R¹⁴ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
A is OH; and
V, Y, and Z are all carbon.

15. The compound of claim 1 selected from
trans-2-{([4′-({[(4-fluorophenyl)amino]carbonyl}amino)biphenyl-4-yl]carbonyl}cyclopentanecarboxylic acid;
trans-2-{[4′-({[(4-ethylphenyl)amino]carbonyl}amino)biphenyl-4-yl]carbonyl}-cyclopropanecarboxylic acid;
trans-2-([4′-({[(4-ethylphenyl)amino]carbonyl}amino)biphenyl-4-yl]carbonyl)-cyclohexanecarboxylic acid;
trans-2-{[4′-({[(4-ethoxyphenyl)amino]carbonyl}amino)biphenyl-4-yl]carbonyl}-cyclopentanecarboxylic acid; and
trans-2-[4′-({[(3,4-dimethylphenyl)amino]carbonyl}amino)biphenyl-4-yl]carbonyl}-cyclopropanecarboxylic acid.

16. The compound of claim 1 selected from
trans-2-([4′-({[(2-chlorophenyl)amino]carbonyl}amino)biphenyl-4-yl]carbonyl}-cyclopropanecarboxylic acid;
trans-2-{[4′-({[(2,4-difluorophenyl)amino]carbonyl}amino)biphenyl-4-yl]carbonyl}-cyclobutanecarboxylic acid;
trans-2-[(4′-{[(3,5-difluorophenyl)acetyl]amino}biphenyl-4-yl)carbonyl]cyclopentanecarboxylic acid;
trans-2-[(4′-{[(3,4-dimethoxyphenyl)acetyl]amino}biphenyl-4-yl)carbonyl]cyclopentanecarboxylic acid; and
trans-2-({4′-[(4-fluoro-3-methylbenzoyl)amino]biphenyl-4-yl}carbonyl)cyclopentanecarboxylic acid.

17. The compound of claim 1 selected from
trans-2-({4′-[(4-ethoxybenzoyl)amino]biphenyl-4-yl}carbonyl)cyclopentanecarboxylic acid;
trans-2-({4′-[(4-butylbenzoyl)amino]biphenyl-4-yl}carbonyl)cyclopentanecarboxylic acid;
trans-2-({4′-[(4-butylbenzoyl)amino]biphenyl-4-yl}carbonyl)cyclopropanecarboxylic acid;
trans-2-({4′-[(3,4-dimethylbenzoyl)amino]biphenyl-4-yl}carbonyl)cyclopentanecarboxylic acid; and
trans-2-({4′-[(3,4-dichlorobenzoyl)amino]biphenyl-4-yl}carbonyl)cyclohexanecarboxylic acid.

18. The compound of claim 1 selected from
4-[4′-({[(4-ethylphenyl)amino]carbonylamino)biphenyl-4-yl]-2,2-dimethyl-4-oxobutanoic acid;
4-[4′-({[(4-ethylphenyl)amino]carbonyl}amino)-3′-fluorobiphenyl-4-yl]-2,2-dimethyl-4-oxobutanoic acid;
4-[4′-({[(3,4-dimethylphenyl)amino]carbonylamino)biphenyl-4-yl]-2,2-dimethyl-4-oxobutanoic acid;
4-[4′-([(3,4-dimethylphenyl)amino]carbonyl}amino)-3′-fluorobiphenyl-4-yl]-2,2-dimethyl-4-oxobutanoic acid; and
4-[4′-({[(2,4-difluorophenyl)amino]carbonyl}amino)-3′-fluorobiphenyl-4-yl]-2,2-dimethyl-4-oxobutanoic acid.

19. The compound of claim 1 selected from
4-[3′-fluoro-4′-({[(4-fluorophenyl)amino]carbonyl}amino)biphenyl-4-yl]-2,2-dimethyl-4-oxobutanoic acid;
(1R,2R)-2-{[4′-({[(4-ethylphenyl)amino]carbonyl}amino)biphenyl-4-yl]carbonyl}-cyclohexanecarboxylic acid;
(1R,2R)-2-[(4′-{[(4-ethoxyphenyl)acetyl]amino}biphenylyl)carbonyl]cyclopentanecarboxylic acid;
(1R,2R)-2-[(4′-{[(3,5-difluorophenyl)acetyl]amino}biphenylyl)carbonyl]cyclopentanecarboxylic acid;
(1R,2R)-2-({4′-[(4-fluoro-3-methylbenzoyl)amino]biphenyl-4-yl}carbonyl)cyclopentanecarboxylic acid; and
(1R,2R)-2-({4′-[(4-ethoxybenzoyl)amino]biphenyl-4-yl}carbonyl)cyclopentanecarboxylic acid.

20. A pharmaceutical composition comprising a therapeutically effective amount of a compound of claim 1, or a pharmaceutically acceptable salt or ester, in combination with a pharmaceutically acceptable carrier.

21. A pharmaceutical composition comprising a therapeutically effective amount of a compound of claim 1, or a pharmaceutically acceptable salt or ester thereof, in combination with a pharmaceutically acceptable carrier and one or more pharmaceutical agents.

22. The pharmaceutical composition of claim 21, wherein said pharmaceutical agent is an anti-obesity agent selected from the group consisting of β-3 adrenergic receptor agonists, cannabinoid antagonists, neuropeptide-Y receptor antagonists, neuropeptide Y5 inhibitors, apo-B/MIP inhibitors, 11β-hydroxy steroid dehydrogenase-1 inhibitors, peptide YY₃₋₃₆, peptide YY₃₋₃₆ analogs, MCR4 agonists, CCK-A agonists, monoamine reuptake inhibitors, sympathomimetic agents, dopamine agonists, melanocyte-stimulating hormone receptor analogs, melanin concentrating hormone antagonists, leptin, leptin analogs, leptin receptor agonists, galanin antagonists, lipase inhibitors, bombesin agonists, thyromimetic agents, dehydroepiandrosterone, dehydroepiandrosterone analogs, glucocorticoid receptor antagonists, orexin receptor antagonists, ciliary neurotrophic factor, ghrelin receptor antagonists, histamine-3 receptor antagonists, neuromedin U receptor agonists, appetite suppressants, modulators of digestion and/or metabolism, thermogenesis modulators, lipolysis modulators, gut motility modulators, fat absorption modulators, and satiety modulators.

23. The pharmaceutical composition of claim 21, wherein said pharmaceutical agent is an agent for the treatment of diabetes selected from the group consisting of insulin, insulin derivatives, PPAR ligands, sulfonylurea drugs, α-glucosidase inhibitors, biguanides, PTP-1B inhibitors, DPP-IV inhibitors, 11-beta-HSD inhibitors, GLP-1 and GLP-1 derivatives, GIP and GIP derivatives, PACAP and PACAP derivatives, and secretin and secretin derivatives.

24. The pharmaceutical composition of claim 21, wherein said pharmaceutical agent is an agent for the treatment of lipid disorders selected from the group consisting of HMG-CoA inhibitors, nicotinic acid, fatty acid lowering compounds, lipid lowering drugs, ACAT inhibitors, bile sequestrants, bile acid reuptake inhibitors, microsomal triglyceride transport inhibitors, and fibric acid derivatives.

25. The pharmaceutical composition of claim 21, wherein said pharmaceutical agent is an anti-hypertensive agent selected from the group consisting of β-blockers, calcium channel blockers, diuretics, renin inhibitors, ACE inhibitors, AT-1 receptor antagonists, ET receptor antagonists, and nitrates.

26. A pharmaceutical composition comprising a therapeutically effective amount of a compound of any of claim 2 to 19, or a pharmaceutically acceptable salt or ester, in combination with a pharmaceutically acceptable carrier.

27. A pharmaceutical composition comprising a therapeutically effective amount of a compound of any of claim 2 to 19, or a pharmaceutically acceptable salt or ester thereof, in combination with a pharmaceutically acceptable carrier and one or more pharmaceutical agents.

28. A method of treating obesity comprising the step of administering to a subject in need thereof a therapeutically effective amount of a compound of claim 1 or a composition of claim 20.

29. A method of inducing weight loss comprising the step of administering to a subject in need thereof a therapeutically effective amount of a compound of claim 1 or a composition of claim 20.

30. A method of preventing weight gain comprising the step of administering to a subject in need thereof a therapeutically effective amount of a compound of claim 1 or a composition of claim 20.

31. A method of treating obesity-related disorders comprising the step of administering to a subject in need thereof a therapeutically effective amount of a compound of claim 1 or a composition of claim 20.

32. The method of claim 31, wherein said obesity-related disorder is selected from the group consisting of dyslipidemia, cholesterol gallstones, gallbladder disease, gout, cancer, menstrual abnormalities, infertility, polycystic ovaries, osteoarthritis, sleep apnea, hypertriglyceridemia, Syndrome X, type 2 diabetes, atherosclerotic diseases, hyperlipidemia, hypercholesteremia, low HDL levels, hypertension, cardiovascular disease, coronary heart disease, coronary artery disease, cerebrovascular disease, stroke, and peripheral vessel disease.

33. A method of treating obesity comprising the step of administering to a subject in need thereof a therapeutically effective amount of a compound of claim 1 in combination with one or more pharmaceutical agents.

34. The method of claim 33, wherein the compound of claim 1 and one or more pharmaceutical agents are administered as a single pharmaceutical dosage formulation.

35. A method of treating obesity comprising the step of administering to a subject in need thereof a therapeutically effective amount of a composition of any of claims 21 to 27.

36. A method of treating obesity-related disorders comprising the step of administering to a subject in need thereof a therapeutically effective amount of a composition of any of claims 21 to 27.

37. Compounds according to claim 1 for the treatment and/or prophylaxis of obesity and obesity-related disorders.

38. Medicament containing at least one compound according to claim 1 in combination with at least one pharmaceutically acceptable, pharmaceutically safe carrier or excipient.

39. Use of compounds according to claim 1 for manufacturing a medicament for the treatment and/or prophylaxis of obesity and obesity-related disorders.

40. Medicaments according to claim 38 for the treatment and/or prophylaxis of obesity.
